(19) United States
(12) Patent Application Publication
(10) Pub. No.: US 2026/0347445 A1
(43) Pub. Date: Nov. 5, 2026

(54) DOSAGE AND ADMINISTRATION OF ANTI-C5 ANTIBODIES FOR PREVENTING OR MINIMIZING CARDIAC SURGERY ASSOCIATED ACUTE KIDNEY INJURY (CSA-AKI) AND/OR SUBSEQUENT MAJOR ADVERSE KIDNEY EVENTS (MAKE) IN PATIENTS WITH CHRONIC KIDNEY DISEASE

(71) Applicant: Alexion Pharmaceuticals, Inc., Boston, MA (US)

(72) Inventor: William Thomas Smith, Woodstock, NY (US)

(21) Appl. No.: 19/115,294

(22) PCT Filed: Sep. 26, 2023
(86) PCT No.: PCT/US2023/033718

§ 371 Date: Mar. 26, 2025

Related U.S. Application Data
(60) Provisional application No. 63/410,753, filed on Sep. 28, 2022.
(60) Provisional application No. 63/440,968, filed on Jan. 25, 2023.

(51) Int. Cl. C07K 16/18; A61K 39/00; A61P 13/12

(52) U.S. Cl. CPC C07K 16/18; A61P 13/12; A61K 2039/545; C07K 2317/52; C07K 2317/565; C07K 2317/72; C07K 2317/76; C07K 2317/92; C07K 2317/94

(57) ABSTRACT

Provided herein are methods of preparing a human patient for surgery (e.g., cardiac surgery with cardiopulmonary bypass (CPB)) in a particular sub-population (e.g., a human patient with kidney disease, including chronic kidney disease (CKD)), methods of inhibiting terminal complement activation in the human patient, methods of treating a human patient with CKD prior to cardiac surgery with CPB, methods of preventing or reducing cardiac surgery associated acute kidney injury (CSA-AKI) in a human patient with CKD, and methods of preventing or reducing one or more MAKE in a human patient with CKD. The methods comprise administering to the patient an anti-C5 antibody, e.g., ravulizumab or eculizumab, or antigen binding fragments thereof, according to a particular clinical dosage regimen and according to a specific schedule.

### CROSS-REFERENCE TO RELATED APPLICATIONS

This application claims priority to, and the benefit of, U.S. Provisional Application No. 63/410,753 (filed Sep. 28, 2022) and U.S. Provisional Application No. 63/440,968 (filed Jan. 25, 2023). The entire contents of the aforementioned applications are incorporated herein by reference.

### BACKGROUND

The presence of chronic kidney disease (CKD) in patients undergoing cardiac surgery with cardiopulmonary bypass (CPB) confers high risk for poor outcomes, and postoperative acute kidney injury (AKI) further enhances these risks (see. e.g., Meersch M. et al., Current Opinion in Anaesthesiology. 2016; 29(3):413-420; Mehta R H, et al., Circulation. 2006; 114(21):2208-2216; Nashef S A, et al. EuroSCORE II. Eur J Cardiothorac Surg. 2012; 41(4):734-744; Thakar C V, et al., Kidney Int. 2005; 67(3):1112-1119; and Thakar C V, et al., J Am. Soc. Nephrol. 2005; 16(1):162-168). AKI after CPB is characterized by an abrupt deterioration in the kidney's excretory function following cardiac surgery, typically manifesting as a reduction in glomerular filtration rate (GFR). AKI occurs in approximately 25% of patients after surgery with CPB (see, e.g., Corredor C, et al., J. Cardiothorac. Vasc. Anesth. 2016; 30(1):69-75; and Hu J. et al., J Cardiothorac. Vasc. Anesth. 2016; 30(1):82-89), and up to 60% to 80% of patients with moderate to severe CKD (calculated from Priyanka P, et al., J. Thorac. Cardiovasc. Surg. 2021; 162(1):143-151.e147).

AKI after CPB is associated with in-hospital mortality, need for kidney replacement therapy (KRT; also known as renal replacement therapy or RRT), permanent loss of kidney function (risking progression of CKD, including to end-stage kidney disease), high resource utilization, and poor long-term survival (see, e.g., Peng et al., Anesth. Analg. 2022 Oct. 1; 135(4):744-756; Chawla L S, et al., N. Engl. J. Med. 2014; 371(1):58-66; Chawla L S, et al., Kidney Int. 2011; 79(12):1361-1369; Coca S G, et al., Am. J. Kidney Dis. 2009; 53(6):961-973; Dasta J F. et al., Nephrology, Dialysis, Transplantation: Official Publication of the European Dialysis and Transplant Association—European Renal Association; 2008; 23(6): 1970-1974; Ishani A, et al., J. Am. Soc. Nephrol. 2009; 20(1):223-228: Mangano C M, et al., The Multicenter Study of Perioperative Ischemia Research Group. Ann Intern Med. 1998; 128(3):194-203; Thakar C V. et al., Incidence and outcomes of acute kidney injury in intensive care units: a Veterans Administration study. Crit Care Med. 2009:37(9):2552-2558). One large meta-analysis showed that in-hospital mortality rates after cardiac surgery for patients without AKI compared to patients with AKI were 1.7% and 10.7%, respectively. The long-term (1-5 years) mortality rates were 11.9% and 30%, respectively (Hu, 2016). Sustained kidney dysfunction (SKD) after AKI increases short- and long-term mortality risk by multifold, compared to transient AKI or no AKI (see, e.g., Brown J R, et al., Ann. Thorac. Surg. 2010; 90(4):1142-1148; Corredor C, et al., J Cardiothorac. Vase. Anesth. 2016; 30(1):69-75; Swaminathan M, et al., Ann. Thorac. Surg. 2010; 89(4):1098-1104) and increases risk of new onset CKD or accelerates progression of pre-existing CKD (see, e.g., Chawla L S, et al., N. Engl. J. Med. 2014; 371(1):58-66; Chawla L S, et al., Kidney Int. 2011; 79(12):1361-1369; and Kellum J A, et al., Nat. Rev. Dis. Primers. 2021; 7(1):52). The risk of developing end-stage kidney disease after AKI is substantially greater in patients with pre-existing CKD (see, e.g., Ishani A. et al., J. Am. Soc. Nephrol. 2009; 20(1):223-228.2009 and Wu V C, et al., Kidney Int. 2011; 80(11):1222-1230).

The need for KRT due to AKI after CPB occurs in about 2% of patients undergoing cardiac surgery (see, e.g., Hu J, et al., J. Cardiothorac. Vase. Anesth. 2016; 30(1):82-89), but appears to be increased in those with underlying CKD, 13.3% in severe CKD (see e.g., Chawla L S, et al., J. Am. Soc. Nephrol. 2012; 23(8):1389-1397; Mehta R H. et al., Circulation. 2006; 114(21):2208-2216; Thakar C V, et al., Kidney Int. 2005; 67(3):1112-1119; Thakar C V, et al., J. Am. Soc. Nephrol. 2005; 16(1):162-168). The in-hospital mortality is extraordinarily high, 33% to >50% (see e.g., Chawla L S, et al., J. Am. Soc. Nephrol. 2012; 23(8):1389-1397; Dasta J F. et al., Nephrology. Dialysis, Transplantation: Official Publication of the European Dialysis and Transplant Association—European Renal Association. 2008; 23(6):1970-1974; Landoni G, et al., European Journal of Anaesthesiology. 2006; 23(1):17-22); and Thakar C V, et al., Kidney Int. 2005; 67(3):1112-1119; Thakar C V, et al., J Am. Soc. Nephrol. 2005; 16(1):162-168).

Multiple treatment approaches have failed to demonstrate a reduction in AKI after CPB or in the poor clinical outcomes related to AKI, i.e., SKD at risk of progressive CKD, need for KRT, and mortality (see, e.g., Schurle A, et al., J. Clin. Med. 2021; 10(24)). There remains a high unmet medical need to develop treatments that reduce the risk of AKI and its consequences in patients with CKD undergoing cardiac surgery with CPB. Accordingly, it is an object of the present disclosure to provide improved methods for preventing and/or minimizing cardiac surgery associated kidney injury (CSA-AKI) and or subsequent major adverse kidney events (MAKE) in patients with CKD.

### SUMMARY

The present disclosure relates to compositions and methods for the prevention and/or treatment of acute kidney injury (AKI) in human patients with chronic kidney disease (CKD) who are undergoing cardiopulmonary bypass (CPB). The present methods improve upon existing therapeutic modalities, such as interoperative strategies and post-operative strategies, which are provided in-patient setting and target the inflammatory mediators of AKI. In contrast to prevailing methods for reducing AKI, the compositions and methods of instant disclosure can be administered in the out-patient setting and are effective in preventing AKI and greatly improve clinical outcomes, e.g., reduce major adverse events and even death, in patients with unmet need.

Provided herein are methods involving preparing a human patient for surgery (e.g., cardiac surgery with cardiopulmonary bypass (CPB)) in a particular sub-population (e.g., a human patient with kidney disease, including chronic kidney disease (CKD)), comprising administering to the patient an anti-C5 antibody, or antigen binding fragment thereof, wherein the anti-C5 antibody, or antigen binding fragment thereof, is administered (or is for administration) according to a particular clinical dosage regimen (e.g., at a single weight-based dose), and according to a specific schedule (e.g., at least one to seven calendar days before the surgery).

Also, provided herein are methods of inhibiting terminal complement activation in a human patient, methods of treating a human patient with CKD prior to cardiac surgery with CPB, methods of preventing or reducing (e.g., minimizing) cardiac surgery associated acute kidney injury (CSA-AKI) in a human patient with CKD, and methods of preventing or reducing (e.g., minimizing) one or more major adverse kidney events (MAKE) in a human patient with CKD.

Any suitable anti-C5 antibody, or antigen binding fragment thereof, can be used in the methods described herein. An exemplary anti-C5 antibody is ravulizumab (ULTOMIRIS®) comprising the heavy and light chains having the sequences shown in SEQ ID NOs:14 and 11, respectively, or antigen binding fragments and variants thereof. In other embodiments, the antibody comprises the heavy and light chain complementarity determining regions (CDRs) or variable regions (VRs) of ravulizumab. Accordingly, in one embodiment, the antibody comprises the CDR1, CDR2 and CDR3 domains of the heavy chain variable (VH) region of ravulizumab having the sequence shown in SEQ ID NO:12, and the CDR1, CDR2 and CDR3 domains of the light chain variable (VL) region of ravulizumab having the sequence shown in SEQ ID NO:8. In another embodiment, the antibody comprises CDR1. CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively.

In another embodiment, the antibody comprises VH and VL regions having the amino acid sequences set forth in SEQ ID NO:12 and SEQ ID NO:8, respectively. In another embodiment, the antibody comprises a heavy chain constant region as set forth in SEQ ID NO:13.

In another embodiment, the antibody comprises a variant human Fc constant region that binds to human neonatal Fc receptor (FcRn), wherein the variant human Fc CH3 constant region comprises Met429Leu and Asn435Ser substitutions at residues corresponding to methionine 428 and asparagine 434 of a native human IgG Fc constant region, each according to the EU numbering convention.

In another embodiment, the antibody comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively and a variant human Fc constant region that binds to human neonatal Fc receptor (FcRn), wherein the variant human Fc CH3 constant region comprises Met429Leu and Asn435Ser substitutions at residues corresponding to methionine 428 and asparagine 434 of a native human IgG Fc constant region, each according to the EU numbering convention.

In another embodiment, the anti-C5 antibody comprises the heavy and light chain CDRs or variable regions of the BNJ421 antibody (described in WO2015134894 and U.S. Pat. No. 9,079,949). In another embodiment, the anti-C5 antibody comprises the heavy and light chain CDRs or variable regions of the 7086 antibody (see U.S. Pat. Nos. 8,241,628 and 8,883,158). In another embodiment, the anti-C5 antibody comprises the heavy and light chain CDRs or variable regions of the 8110 antibody (see U.S. Pat. Nos. 8,241,628 and 8,883,158). In another embodiment, the anti-C5 antibody comprises the heavy and light chain CDRs or variable regions of the 305LO5 antibody (see U.S. Pat. No. 9,765,135). In another embodiment, the anti-C5 antibody comprises the heavy and light chain CDRs or variable regions of the SKY59 antibody. In another embodiment, the anti-C5 antibody comprises the heavy and light chain CDRs or variable regions of the REGN3918 antibody.

In another embodiment, the anti-C5 antibody is a biosimilar of eculizumab (SOLIRIS®). For example, in one embodiment, the anti-C5 antibody is, for example, ABP 959 antibody (manufactured by Amgen Inc., USA), ELIZARIA® (manufactured by Generium JNC, Russia), SB12 (manufactured by Samsung Bioepis, Incheon, South Korea), ISU305 (eculizumab biosimilar from ISU Abxis, South Korea), ABLYZEE (eculizumab biosimilar from CinnaGen, Iran), BCD 148 (eculizumab biosimilar from Biocad Medical, Quebec, Canada), tesidolumab (manufactured by Novartis), Crovalimab (manufactured by Roche), CAN106 (manufactured by CanBridge Pharmaceuticals, China) or Pozelimab (manufactured by Regeneron).

In another embodiment, the antibody competes for binding with, and/or binds to the same epitope on C5 as any of the above-mentioned antibodies. In another embodiment, the antibody has at least about 90% variable region amino acid sequence identity to any of the above-mentioned antibodies (e.g. at least about 90%, 95% or 99% variable region identity with SEQ ID NO:12 or SEQ ID NO:8).

In another embodiment, the antibody binds to human C5 at pH 7.4 and 25 °C with an affinity dissociation constant (K_{D}) that is in the range 0.1 nM≤K_{D}≤1 nM. In another embodiment, the antibody binds to human C5 at pH 7.4 and 25 °C with an affinity dissociation constant (K_{D}) of about 0.5 nM. In another embodiment, the antibody binds to human C5 at pH 6.0 and 25 °C with a K_{D}≥10 nM. In another embodiment, the antibody binds to human C5 at pH 6.0 and 25 °C with a K_{D} of about 22 nM. In yet another embodiment, the [(K_{D} of the antibody or antigen-binding fragment thereof for human C5 at pH 6.0 and at 25 °C)/(K_{D} of the antibody or antigen-binding fragment thereof for human C5 at pH 7.4 and at 25 °C)] of the antibody is greater than 25.

The method described herein can be utilized with any type of cardiac surgery. In another embodiment, the cardiac surgery is a coronary artery bypass graft (CABG). In another embodiment, the surgery is valve replacement or repair. In another embodiment, the surgery is insertion of a pacemaker or an implantable cardioverter defibrillator (ICD). In another embodiment, the surgery is Maze surgery. In another embodiment, the surgery is heart transplant. In another embodiment, the surgery is insertion of a ventricular assist device (VAD). In another embodiment, the surgery is insertion of a total artificial heart (TAH). In another embodiment, the surgery is insertion of a transcatheter structural heart surgery. In a particular embodiment, the surgery is cardiac surgery with CPB.

In one embodiment, the anti-C5 antibody, or antigen binding fragment thereof, is administered to the patient at least one calendar day prior to the surgery (e.g., cardiac surgery with CPB). In another embodiment the anti-C5 antibody, or antigen binding fragment thereof, is administered from one to seven calendar days prior to the surgery. For example, in one embodiment, the anti-C5 antibody, or antigen binding fragment thereof, is administered one, two, three, four, five, six, or seven calendar days prior to the surgery.

The anti-C5 antibody, or antigen binding fragment thereof, is administered to the patient according to the methods described herein at a weight-based dose (e.g., a single pre-operative weight-based dose). In one embodiment, the anti-C5 antibody, or antigen binding fragment thereof, is administered at a dose of 2700 mg to a patient weighing ≥30 to <40 kg. In another embodiment, the anti-C5 antibody, or antigen binding fragment thereof, is administered at a dose of 3000 mg to a patient weighing ≥40 to <60 kg. In another embodiment, the anti-C5 antibody, or antigen binding fragment thereof, is administered at a dose of 3300 mg to a patient weighing ≥60 to <100 kg. In another embodiment, the anti-C5 antibody, or antigen binding fragment thereof, is administered at a dose of 3600 mg to a patient weighing ≥100 kg.

In one aspect, a method of preparing a human patient (e.g., a human patient with kidney disease, including CKD) for surgery (e.g., cardiac surgery with CPB) is provided, wherein the method comprises administering to the patient an effective amount of an anti C5 antibody, or antigen binding fragment thereof, comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, wherein the anti C5 antibody, or antigen binding fragment thereof, is administered once prior to the surgery.

In one embodiment, a method of preparing a human patient with CKD for cardiac surgery with CPB is provided, wherein the method comprises administering to the patient an effective amount of an anti C5 antibody, or antigen binding fragment thereof, comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, wherein the anti C5 antibody, or antigen binding fragment thereof, is administered prior to the surgery once at a dose of:
a) 2700 mg to a patient weighing ≥30 to <40 kg;
b) 3000 mg to a patient weighing ≥40 to <60 kg;
c) 3300 mg to a patient weighing ≥60 to <100 kg; or
d) 3600 mg to a patient weighing ≥100 kg.

In another aspect, a method of inhibiting terminal complement activation in a human patient (e.g., a human patient with kidney disease, including CKD) prior to surgery (e.g., cardiac surgery with CPB) is provided, wherein the method comprises administering to the patient an effective amount of an anti C5 antibody, or antigen binding fragment thereof, comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, wherein the anti C5 antibody, or antigen binding fragment thereof, is administered once prior to the surgery.

In one embodiment, a method of inhibiting terminal complement activation in a human patient with CKD prior to cardiac surgery with CPB is provided, wherein the method comprises administering to the patient an effective amount of an anti C5 antibody, or antigen binding fragment thereof, comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, wherein the anti C5 antibody, or antigen binding fragment thereof, is administered prior to the surgery once at a dose of:
a) 2700 mg to a patient weighing ≥30 to <40 kg;
b) 3000 mg to a patient weighing ≥40 to <60 kg;
c) 3300 mg to a patient weighing ≥60 to <100 kg; or
d) 3600 mg to a patient weighing ≥100 kg.

In some embodiments, terminal complement activation is inhibited in a human patient, according to the methods described herein, as assessed by any suitable assay. In one embodiment, the method inhibits terminal complement activation in a human patient, for example, by 50%, 55%, 60%, 65%, 70%, 75%, 80%, 85%, 86%, 87%, 88%, 89%, 90%, 91%, 92%, 93%, 94%, 95%, 96%, 97%, 98%, 99%, or 100%.

In another aspect, a method of treating a human patient with kidney disease (e.g., CKD) prior to cardiac surgery (e.g., with CPB), is provided, wherein the method comprises administering to the patient an effective amount of an anti-C5 antibody, or antigen binding fragment thereof, comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, wherein the anti-C5 antibody, or antigen binding fragment thereof, is administered once prior to the surgery.

In one embodiment, a method of treating a human patient with CKD prior to cardiac surgery with CPB, is provided, wherein the method comprises administering to the patient an effective amount of an anti-C5 antibody, or antigen binding fragment thereof, comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, wherein the anti-C5 antibody, or antigen binding fragment thereof, is administered prior to the surgery once at a dose of:
a) 2700 mg to a patient weighing ≥30 to <40 kg;
b) 3000 mg to a patient weighing ≥40 to <60 kg;
c) 3300 mg to a patient weighing ≥60 to <100 kg; or
d) 3600 mg to a patient weighing ≥100 kg.

In another aspect, a method of preventing or reducing (e.g., minimizing) CSA-AKI in a human patient with kidney disease (e.g., CKD), wherein the method comprises administering to the patient an effective amount of an anti-C5 antibody, or antigen binding fragment thereof, wherein the anti-C5 antibody, or antigen binding fragment thereof, comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, and wherein the anti-C5 antibody, or antigen binding fragment thereof, is administered once prior to cardiac surgery (e.g., with CPB).

In one embodiment, a method of preventing or reducing (e.g., minimizing) CSA-AKI in a human patient with CKD is provided, wherein the method comprises administering to the patient an effective amount of an anti-C5 antibody, or antigen binding fragment thereof, wherein the anti-C5 antibody, or antigen binding fragment thereof, comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, and wherein the anti-C5 antibody, or antigen binding fragment thereof, is administered prior to cardiac surgery (e.g., with CPB) once at a dose of:
a) 2700 mg to a patient weighing ≥30 to <40 kg;
b) 3000 mg to a patient weighing ≥40 to <60 kg;
c) 3300 mg to a patient weighing ≥60 to <100 kg; or
d) 3600 mg to a patient weighing ≥100 kg.

In another aspect, a method of preventing or reducing (e.g., minimizing) one or more MAKE in a human patient with kidney disease (e.g., CKD), wherein the method comprises administering to the patient an effective amount of an anti-C5 antibody, or antigen binding fragment thereof, wherein the anti-C5 antibody, or antigen binding fragment thereof, comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, and wherein the anti-C5 antibody, or antigen binding fragment thereof, is administered once prior to cardiac surgery (e.g., with CPB).

In one embodiment, a method of preventing or reducing (e.g., minimizing) one or more MAKE in a human patient with CKD is provided, wherein the method comprises administering to the patient an effective amount of an anti-C5 antibody, or antigen binding fragment thereof, wherein the anti-C5 antibody, or antigen binding fragment thereof, comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, and wherein the anti-C5 antibody, or antigen binding fragment thereof, is administered prior to cardiac surgery (e.g., with CPB), once at a dose of:
a) 2700 mg to a patient weighing ≥30 to <40 kg;
b) 3000 mg to a patient weighing ≥40 to <60 kg;
c) 3300 mg to a patient weighing ≥60 to <100 kg; or
d) 3600 mg to a patient weighing ≥100 kg.

In certain embodiments, the methods described herein provide an optimal desired response (e.g., inhibits terminal complement activation in a human patient (e.g., a human patient with kidney disease, including CKD) prior to surgery (e.g., cardiac surgery with CPB), prevents or reduces CSA-AKI in a human patient with kidney disease (e.g., CKD) having surgery (e.g., cardiac surgery with CPB), and/or prevents or reduces one or more MAKE in a human patient with kidney disease (e.g., CKD) having surgery (e.g., cardiac surgery with CPB)).

In certain embodiments, the methods described herein are sufficient to maintain particular serum trough concentrations of the anti-C5 antibody, or antigen binding fragment thereof. In one embodiment, for example, the method maintains a serum trough concentration of the anti-C5 antibody, or antigen binding fragment thereof, of 50, 55, 60, 65, 70, 75, 80, 85, 90, 95, 100, 105, 110, 115, 120, 125, 130, 135, 140, 145, 150, 155, 160, 165, 170, 175, 180, 185, 190, 200, 205, 210, 215, 220, 225, 230, 240, 245, 250, 255, 260, 265, 270, 280, 290, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465.470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700 μg/mL or greater. In one embodiment, the method maintains a serum trough concentration of the anti-C5 antibody, or antigen binding fragment thereof, of 100 μg/mL or greater, 150 μg/mL or greater, 200 μg/mL or greater, 250 μg/mL or greater, 300 μg/mL or greater, 350 μg/mL or greater, 400 μg/mL or greater, or 450 μg/mL or greater. In another embodiment, the method maintains a serum trough concentration of the anti-C5 antibody, or antigen binding fragment thereof, of between 100 μg/mL and 700 μg/mL, preferably between 300 μg/mL and 600 μg/mL. In another embodiment, the method maintains a serum trough concentration of the anti-C5 antibody, or antigen binding fragment thereof, of about 475 μg/mL. In another embodiment, the method maintains a serum peak concentration of the anti-C5 antibody, or antigen binding fragment thereof, of less than about 1800, 1780, 1760, 1740, 1720, 1700, 1680, 1660, 1640, 1620, 1600, 1580, 1560, 1540, 1520, 1500, 1480, 1460, 1440, 1420, 1400, 1380, 1360, 1340, 1320, 1300, 1280, 1260, 1240, 1220, 1200, 1180, 1160, 1140, 1120, 1100, 1080, 1060, 1040, 1020, 1000, 980, 960, 940, 920, or 900 μg/mL, or less. In other embodiments, the method maintains a peak serum concentration of the anti-C5 antibody, or antigen binding fragment thereof, of between 900 μg/mL and 1800 μg/mL, preferably between 1050 μg/mL and 1550 μg/mL. In another embodiment, the method maintains a peak serum concentration of the anti-C5 antibody, or antigen binding fragment thereof, of about 1350 μg/mL.

In another embodiment, the anti-C5 antibody, or antigen binding fragment thereof, is administered to the patient in an amount and with a frequency to maintain at least 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg, 200 μg, 205 μg, 210 μg, 215 μg, 220 μg, 225 μg, 230 μg, 235 μg, 240 μg, 245 μg, 250 μg, 255 μg, 260 μg, 270 μg, 280 μg, 290 μg, 300 μg, 320 μg, 340 μg, 360 μg, 380 μg, 400 μg, 420 μg, 440 μg, 460 μg, 480 μg, 500 μg, 550 μg, 600 μg, 650 μg, 700 μg, 750 μg, 800 μg, 850 μg, 900 μg, 950 μg, 1000 μg, 1050 μg, 1100 μg, 1150 μg, 1200 μg, 1250 μg, 1300 μg, 1350 μg, 1400 μg, 1450 μg, 1500 μg, 1550 μg, 1600 μg, 1650 μg, 1700 μg, 1750 μg, or more, e.g., 1800 μg of antibody per milliliter of the patient's blood.

In another embodiment, anti-C5 antibody, or antigen binding fragment thereof, is administered to the patient in an amount and with a frequency to maintain a minimum free C5 concentration. In one embodiment, for example, the anti-C5 antibody, or antigen binding fragment thereof, is administered to the patient in an amount and with a frequency to maintain a free C5 concentration of 0.5 μg/mL or less (e.g., 0.4 μg/mL, 0.3 μg/mL, 0.2 μg/mL, or 0.1 μg/mL or less).

The anti-C5 antibodies, or antigen binding fragments thereof, can be administered to a patient by any suitable means. In one embodiment, the antibodies are formulated for intravenous administration.

The efficacy of the methods provided herein can be assessed using any suitable means. In one embodiment, the single pre-operative weight-based dose of the anti-C5 antibody, or antigen binding fragment thereof, results in at least 18 days of complete C5 inhibition.

In another embodiment, the method prevents the need for kidney replacement therapy (KRT).

In another embodiment, the method prevents or reduces CSA-AKI in the human patient with CKD. In one embodiment, CSA-AKI is characterized by an increase in:
a) serum creatinine (sCr) or serum Cystatin C (sCysC) by ≥0.3 mg/dL in a 48-hour period within 7 days following CPB and/or
b) sCr or or sCysC≥1.5 times baseline within 7 days following CPB or at Day 15, 30, 60 or 90 post CPB.

In another embodiment, post-treatment, the human patient is free of severe CSA-AKI (Stage 2 or 3) based on highest observed sCr within 7, 30, 45, 60, or 90 days post CPB, as assessed by modified Kidney Disease Improving Global Outcomes (KDIGO) criteria.

In another embodiment, post-treatment, the human patient is free of severe CSA-AKI based on highest

observed sCr, within 7, 30, 45, 60, or 90 days post CPB, as assessed by Modified “Risk, Injury, Failure, Loss of kidney function, and End-stage kidney disease” (RIFLE) Criteria.

In another embodiment, the method results in a complete recovery from CSA-AKI within 7, 30, 45, 60, or 90 days post-surgery, wherein the complete recovery is characterized by sCr<1.1×baseline.

In another embodiment, the method results in a partial recovery from CSA-AKI within 7, 30, 45, 60, or 90 days post-surgery, wherein the partial recovery is characterized by sCr≥1.1−<1.5×baseline.

In another embodiment, the method results in an improvement from CSA-AKI within 7, 30, 45, 60, or 90 days post-surgery, wherein the improvement is characterized by sCr≥1.5−<2.0×baseline.

In another embodiment, the method results in stable CSA-AKI within 7, 30, 45, 60, or 90 days post-surgery, characterized by sCr≥2.0−<3.0×baseline.

In another embodiment, the method prevents or reduces one or more MAKE in the human patient with CKD. In one embodiment, the one or more MAKE is sustained kidney dysfunction (SKD) defined as an estimated glomerular filtration rate (eGFR)>25% below baseline post CPB, for example, wherein the decrease in eGFR is determined by the Chronic Kidney Disease Epidemiology Collaboration (CKD-EPI) formula based on serum Cystatin C (sCysC) or serum creatinine (sCr). In another embodiment, the one or more MAKE is the occurrence of kidney replacement therapy (KRT) post CPB. In another embodiment, the one or more MAKE is death from any cause post CPB.

In another embodiment, the method results in a change from baseline in quality of life as assessed via a Quality of Life Assessment. For example, in one embodiment, the Quality of Life Assessment is a Kidney Disease Quality of Life instrument (KDQOL-36). In another embodiment, the Quality of Life Assessment is The European Quality of Life Group's 5 dimension 5-level (EQ-5D-5L). In another embodiment, the Quality of Life Assessment is The Functional Assessment of Chronic Illness Therapy (FACIT) Fatigue scale.

In another embodiment, the method results in a shift toward normal levels of biomarkers associated with vascular inflammation (e.g., shed tumor necrosis factor receptor 1 [TNF-R1 or sTNF-R1]). In another embodiment, the method results in a shift toward normal levels of biomarkers associated with endothelial damage and/or activation (e.g., thrombomodulin). In another embodiment, the method results in a shift toward normal levels of biomarkers associated with renal injury (e.g., neutrophil gelatinase-associated lipocalin └NGAL┘). In another embodiment, the method results in a shift toward normal levels of biomarkers associated with inducers of cell-cycle arrest (e.g., tissue inhibitor of metalloproteinase-2 [TIMP-2]). In another embodiment, the method results in a shift toward normal levels of complement proteins and complement activation pathway products (e.g., soluble C5b-9).

Further provided are kits that include a pharmaceutical composition containing an anti-C5 antibody, or antigen binding fragment thereof, such as eculizumab (SOLIRIS®) or ravulizumab (ULTOMIRIS®), and a pharmaceutically acceptable carrier, in a therapeutically effective amount adapted for use in the methods described herein.

In one embodiment, the kit comprises: (a) a dose of an anti-C5 antibody, or antigen binding fragment thereof, CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively and (b) instructions for using the anti-C5 antibody or antigen binding fragment thereof in the methods described herein.

In another aspect, an anti-C5 antibody, or antigen-binding fragment thereof, (e.g., ravulizumab (ULTOMIRIS®)), for use in preparing a human patient with CKD for cardiac surgery with CPB is provided, wherein the anti-C5 antibody, or antigen binding fragment thereof, is administered prior to the surgery once at a dose, e.g., unit dose, of:
a) 2700 mg to a patient weighing ≥30 to <40 kg;
b) 3000 mg to a patient weighing ≥40 to <60 kg;
c) 3300 mg to a patient weighing ≥60 to <100 kg; or
d) 3600 mg to a patient weighing ≥100 kg.

In another embodiment, an anti-C5 antibody, or antigen-binding fragment thereof, for use in inhibiting terminal complement activation in a human patient with CKD prior to cardiac surgery with CPB is provided, wherein the anti-C5 antibody, or antigen binding fragment thereof, is administered prior to the surgery once at a dose, e.g., unit dose, of:
a) 2700 mg to a patient weighing ≥30 to <40 kg;
b) 3000 mg to a patient weighing ≥40 to <60 kg;
c) 3300 mg to a patient weighing ≥60 to <100 kg; or
d) 3600 mg to a patient weighing ≥100 kg.

In another embodiment, an anti-C5 antibody, or antigen-binding fragment thereof, for use in treatment of a human patient with CKD prior to cardiac surgery with CPB is provided, wherein the anti-C5 antibody, or antigen binding fragment thereof, is administered prior to the surgery once at a dose, e.g., unit dose, of:
a) 2700 mg to a patient weighing ≥30 to <40 kg;
b) 3000 mg to a patient weighing ≥40 to <60 kg;
c) 3300 mg to a patient weighing ≥60 to <100 kg; or
d) 3600 mg to a patient weighing ≥100 kg.

In another embodiment, an anti-C5 antibody, or antigen-binding fragment thereof, for use in preventing or reducing CSA-AKI in a human patient with CKD is provided, wherein the anti-C5 antibody, or antigen binding fragment thereof, is administered prior to the surgery once at a dose, e.g., unit dose, of:
a) 2700 mg to a patient weighing ≥30 to <40 kg;
b) 3000 mg to a patient weighing ≥40 to <60 kg;
c) 3300 mg to a patient weighing ≥60 to <100 kg; or
d) 3600 mg to a patient weighing ≥100 kg.

In another embodiment, an anti-C5 antibody, or antigen-binding fragment thereof, for use in preventing or reducing one or more MAKEs in a human patient with CKD is provided, wherein the anti-C5 antibody, or antigen binding fragment thereof, is administered prior to the surgery once at a dose. e.g., unit dose, of:
a) 2700 mg to a patient weighing ≥30 to <40 kg;
b) 3000 mg to a patient weighing ≥40 to <60 kg;
c) 3300 mg to a patient weighing ≥60 to <100 kg; or
d) 3600 mg to a patient weighing ≥100 kg.

In a further aspect, the invention provides for the use of an anti-C5, antibody or antigen-binding fragment thereof (e.g., ravulizumab (ULTOMIRIS®), for preparing a human patient with CKD for cardiac surgery with CPB, wherein the anti-C5 antibody, or antigen binding fragment thereof, is administered prior to the surgery once at a dose, e.g., unit dose, of:
a) 2700 mg to a patient weighing ≥30 to <40 kg;
b) 3000 mg to a patient weighing ≥40 to <60 kg;
c) 3300 mg to a patient weighing ≥60 to <100 kg; or
d) 3600 mg to a patient weighing ≥100 kg.

In one embodiment, the use of an anti-C5, antibody or antigen-binding fragment thereof, for inhibiting terminal complement activation in a human patient with CKD prior to cardiac surgery with CPB is provided, wherein the anti C5 antibody, or antigen binding fragment thereof, is administered prior to the surgery once at a dose, e.g., unit dose, of:
a) 2700 mg to a patient weighing ≥30 to <40 kg;
b) 3000 mg to a patient weighing ≥40 to <60 kg;
c) 3300 mg to a patient weighing ≥60 to <100 kg; or
d) 3600 mg to a patient weighing ≥100 kg.

In another embodiment, the use of an anti-C5, antibody or antigen-binding fragment thereof, for treatment of a human patient with CKD prior to cardiac surgery with CPB is provided, wherein the anti-C5 antibody, or antigen binding fragment thereof, is administered prior to the surgery once at a dose, e.g., unit dose, of:
a) 2700 mg to a patient weighing ≥30 to <40 kg;
b) 3000 mg to a patient weighing ≥40 to <60 kg;
c) 3300 mg to a patient weighing ≥60 to <100 kg; or
d) 3600 mg to a patient weighing ≥100 kg.

In another embodiment, the use of an anti-C5 antibody, or antigen-binding fragment thereof, in preventing or reducing CSA-AKI in a human patient with CKD is provided, wherein the anti-C5 antibody, or antigen binding fragment thereof, is administered prior to the surgery once at a dose, e.g., unit dose, of:
a) 2700 mg to a patient weighing ≥30 to <40 kg;
b) 3000 mg to a patient weighing ≥40 to <60 kg;
c) 3300 mg to a patient weighing ≥60 to <100 kg; or
d) 3600 mg to a patient weighing ≥100 kg.

In another embodiment, the use of an anti-C5 antibody, or antigen-binding fragment thereof, in preventing or reducing one or more MAKEs in a human patient with chronic kidney disease, wherein the anti-C5 antibody, or antigen binding fragment thereof, is administered prior to the surgery once at a dose, e.g., unit dose, of:
a) 2700 mg to a patient weighing ≥30 to <40 kg;
b) 3000 mg to a patient weighing ≥40 to <60 kg;
c) 3300 mg to a patient weighing ≥60 to <100 kg; or
d) 3600 mg to a patient weighing ≥100 kg.

### BRIEF DESCRIPTION OF THE DRAWINGS

FIG. 1 is schematic of the clinical trial protocol.

FIGS. 2A-2G set forth the Schedule of Activities for the clinical trial protocol described in Example 1. The following abbreviations are used in the Schedule of Activities: ADA=antidrug antibody; AE=adverse event; AKI=acute kidney injury; CPB=cardiopulmonary bypass; CT=computerized tomography; D and d=day; ECG=electrocardiogram: ED=early discontinuation; eGFR=estimated glomerular filtration rate; EQ 5D 5L=European Quality of Life Group's 5 dimensions 5-level; FACIT Fatigue=Functional Assessment of Chronic Illness Therapy-Fatigue; FSH=follicle stimulating hormone; ICU=intensive care unit; KDQOL 36=Kidney Disease Quality of Life instrument-36 items; KRT=kidney replacement therapy; PD=pharmacodynamic(s): PK=pharmacokinetic(s); pRBC=packed red blood cells; sCysC=serum cystatin C; RBC=red blood cells; STS=Society of Thoracic Surgeons; and WOCBP=women of childbearing potential.

### DETAILED DESCRIPTION

### I. Definitions

As used herein, the term “subject” or “patient” is a human patient (e.g., a patient having kidney disease, e.g., chronic kidney disease).

As used herein, the term “pediatric” patient is a human patient that has been classified by a physician or caretaker as belonging to a non-adult category and can include. e.g., newborn (both preterm and of term), infants, children, and adolescents. Typically, pediatric patients are patients under 18 years of age (<18 years of age).

As used herein, the term “adult” patient is a human patient that has been classified by a physician or caretaker as such. e.g., one who is not a newborn, infant, child or adolescent, e.g., based on age, developmental status, physiological features, etc. Typically, adult patients are patients who are 18 years of age or older (≥18 years of age).

As used herein, the phrase “chronic kidney disease” (CKD) (also known as chronic renal disease) is a condition characterized by a gradual loss of kidney function over time. Diabetes and high blood pressure, or hypertension, are responsible for two-thirds of chronic kidney disease cases. Other conditions or circumstances that can cause kidney disease include, but are not limited to glomerulonephritis, inherited diseases (such as polycystic kidney disease (PKD)), kidney and urinary tract abnormalities before birth, autoimmune diseases, or other causes such as obstructions caused by kidney stones or tumors, an enlarged prostate gland in men, or repeated urinary infections. Symptoms of CKD include, but are not limited to, feeling tired and having less energy, having trouble concentrating, having a poor appetite, having trouble sleeping, having muscle cramping at night, having swollen feet and ankles, having puffiness around the eyes, having dry, itchy skin, and/or needing to urinate more often, especially at night.

CKD is often diagnosed by one or more of the following tests. One test is an albumin to creatine ratio urine test. Albumin is a protein that shouldn't be found in urine and indicates kidney function problems. Another way to test is a blood test for creatinine. This test determines if there is too much creatinine (a waste product) in the blood. A third option is to test a patient's Glomerular Filtration Rate (GFR). GFR is calculated using the results from the tests and other factors, like age and gender. The result of the GFR is the best way to measure a patient's level of kidney function and to determine the stage of kidney disease.

As used herein, the phrase “major adverse kidney event” (MAKE) refers to: (1) sustained kidney dysfunction (SKD), defined as an estimated glomerular filtration rate (eGFR)>25% below baseline post CPB (e.g., wherein the decrease in eGFR is determined by the Chronic Kidney Disease Epidemiology Collaboration (CKD-EPI) formula based on serum Cystatin C (sCysC) or serum creatinine (sCr)), (2) the occurrence of kidney replacement therapy (KRT) post CPB, and/or (3) death from any cause post CPB.

As used herein, the phrase “cardiac associated acute kidney injury” (CSA-AKI) is an event characterized by an abrupt deterioration in kidney function following cardiac surgery, which is evident from a reduction in GFR. CSA-AKI is the second most common cause of AKI in the intensive care setting and is associated with increased mortality. The pathophysiology of CSA-AKI is very complex and likely includes renal ischemia-reperfusion injury, inflammation, oxidative stress, hemolysis and/or nephrotoxins.

CSA-AKI is characterized by an increase in: serum creatinine (sCr) or serum Cystatin C (sCysC) by ≥0.3 mg/dL in a 48-hour period within 7 days following CPB and/or sCr or or sCysC≥1.5 times baseline within 7 days following CPB. Severe CSA-AKI refers to Stage 2 or 3 according to modified Kidney Disease Improving Global Outcomes (KDIGO) criteria. A human patient is considered free of severe CSA-AKI based on highest observed sCr post-CPB (e.g., within 7, 30, 45, 60, or 90 days post CPB), as assessed by Modified “Risk, Injury, Failure, Loss of kidney function, and End-stage kidney disease” (RIFLE) Criteria. A complete recovery from CSA-AKI is characterized by sCr<1.1×baseline post-CPB (e.g., within 7, 30, 45, 60, or 90 days post CPB). A partial recovery from CSA-AKI is characterized by sCr≥1.1−<1.5×baseline (e.g., within 7, 30, 45, 60, or 90 days post CPB). An improvement from CSA-AKI is characterized by sCr≥1.5−<2.0×baseline (e.g., within 7, 30, 45, 60, or 90 days post CPB). Stable CSA-AKI is characterized by sCr≥2.0−<3.0×baseline (e.g., within 7, 30, 45, 60, or 90 days post CPB).

As used herein, the phrase “cardiac surgery” (also known as cardiovascular surgery or heart surgery), refers to any surgical procedure that involves the heart or the blood vessels that carry blood to and from the heart. Examples of cardiac surgery include, but are not limited to coronary artery bypass graft (CABG), valve replacement or repair, insertion of a pacemaker or an implantable cardioverter defibrillator (ICD), Maze surgery, Maze surgery, heart transplant, and insertion of a ventricular assist device (VAD) or total artificial heart (TAH), and transcatheter structural heart surgery.

CABG (also referred to as heart bypass or coronary artery bypass surgery) is one of the most common types of heart surgery and involves takes a healthy artery or vein from elsewhere in the body and connecting it to supply blood past the blocked coronary artery. The grafted artery or vein bypasses the blocked portion of the coronary artery, creating a new path for blood to flow to the heart muscle. Often, this is done for more than one coronary artery during the same surgery.

In the case of heart valve repair or replacement, the surgeon either repairs the valve or replaces it with an artificial valve or with a biological valve made from pig, cow, or human heart tissue. One repair option is to insert a catheter through a large blood vessel, guide it to the heart, and inflate and deflate a small balloon at the tip of the catheter to widen a narrow valve.

Medicine is usually the first treatment option for arrhythmia, a condition in which the heart beats too fast, too slow or with an irregular rhythm. If medication does not work, a surgeon may implant a pacemaker or an ICD under the skin of the chest or abdomen, with wires that connect it to the heart chambers. The device uses electrical pulses to control the heart rhythm when a sensor detects that it is abnormal. An ICD works similarly, but it sends an electric shock to restore a normal rhythm when it detects a dangerous arrhythmia.

In the case of maze surgery, the surgeon creates a pattern of scar tissue within the upper chambers of the heart to redirect electrical signals along a controlled path to the lower heart chambers. The surgery blocks the stray electrical signals that cause atrial fibrillation, which is the most common type of serious arrhythmia.

During aneurysm repair, a weak section of the artery or heart wall is replaced with a patch or graft to repair a balloon-like bulge in the artery or wall of the heart muscle.

During heart transplant, the diseased heart is removed and replaced with a healthy heart from a deceased donor.

A VAD is a mechanical pump that supports heart function and blood flow. A TAH replaces the two lower chambers of the heart.

In addition to these surgeries, a minimally invasive alternative to open-heart surgery that is becoming more common is transcatheter structural heart surgery. This involves guiding a long, thin, flexible tube called a catheter to your heart through blood vessels that can be accessed from the groin, thigh, abdomen, chest, neck, or collarbone. A small incision is necessary. This type of surgery includes transcatheter aortic valve implantation to replace a faulty aortic valve with a valve made from animal tissue, MitraClip® placement for mitral valve abnormalities, and WATCHMAN® placement for nonvalvular atrial fibrillation patients.

As used herein, “cardiopulmonary bypass” (CPB) refers to the heart-lung machine that is used during cardiac surgery. CPB provides patients with cardiac and pulmonary support, while bypassing the heart and lungs. CPB artificially provides patients three physiologic processes or functions: (1) it adds oxygen to the blood, (2) it pumps or circulates the blood through both the cardiopulmonary bypass circuit and the patient, and (3) it removes excess carbon dioxide from the blood. To accomplish this, the surgeon inserts cannulas into the patient's major veins (typically the superior vena cava and inferior vena cava) and arteries (typically the aorta). Once the cannulas from the patient have been connected to the cardiopulmonary bypass circuit, the blood is drained from the veins into the heart-lung machine while the blood is pumped into an artificial lung (oxygenator), which adds oxygen and removes carbon dioxide. The oxygenated blood is pumped back to the aorta to provide oxygen to the tissues and organs of the patient. CPB may include sternotomy and/or aortic cross-clamping.

As used herein, “effective treatment” refers to treatment producing a beneficial effect, e.g., amelioration of at least one symptom of a disease or disorder. A beneficial effect can take the form of an improvement over baseline, e.g., an improvement over a measurement or observation made prior to initiation of therapy according to the method.

The term “effective a mount” refers to an amount of an agent that provides the desired result (e.g., biological, therapeutic and/or prophylactic result). That result can be prevention, reduction, amelioration, palliation, lessening, delaying and/or alleviation of one or more of events or of the signs, symptoms or causes of a disease, or any other desired alteration of a biological system. An effective amount can be administered in one or more administrations.

As used herein, the term “serum trough level” refers to the lowest level that the agent (e.g., the anti-C5 antibody, or antigen binding fragment thereof) or medicine is present in the serum. In contrast, a “peak serum level.” refers to the highest level of the agent in the serum. The “average serum level.” refers to the mean level of the agent in the serum over time.

The term “antibody” describes a polypeptide comprising at least one antibody-derived antigen binding site (e.g., VH/VL region or Fv, or CDR). Antibodies include known forms of antibodies, e.g., the antibody can be a human antibody, a humanized antibody, a bispecific antibody or a chimeric antibody. The antibody also can be a Fab. Fab′2, ScFv, SMIP, Affibody®, nanobody or a single-domain antibody. The antibody also can be of any of the following isotypes: IgG1, IgG2, IgG3, IgG4, IgM, IgAQ1, IgA2, IgAsec, IgD, IgE or combinations thereof. The antibody can be a naturally occurring antibody or an antibody that has been altered by a protein engineering technique (e.g., by mutation, deletion, substitution, conjugation to a non-antibody moiety). An antibody can include, for example, one or more variant amino acids (compared to a naturally occurring antibody) that change a property (e.g., a functional property) of the antibody. Numerous such alterations are known in the art that affect, e.g., half-life, effector function, and/or immune responses to the antibody in a patient. The term antibody also includes artificial or engineered polypeptide constructs that comprise at least one antibody-derived antigen binding site.

### II. Anti-C5 Antibodies

Anti-C5 antibodies described herein bind to complement component C5 (e.g., human C5) and inhibit the cleavage of C5 into fragments C5a and C5b. As described above, such antibodies also have, for example, improved pharmacokinetic properties relative to other anti-C5 antibodies (e.g., eculizumab) used for therapeutic purposes.

Anti-C5 antibodies (or VH/VL domains derived therefrom) suitable for use in the methods described herein can be generated using methods known in the art. Alternatively, art recognized anti-C5 antibodies can be used. Antibodies that compete for binding to C5 with any of these art recognized antibodies or antibodies described herein can also be used.

An exemplary anti-C5 antibody is ravulizumab comprising heavy and light chains having the sequences shown in SEQ ID NOs:14 and 11, respectively, or antigen binding fragments and variants thereof. Ravulizumab (also known as ULTOMIRIS®, BNJ441 and ALXN1210) is described in WO2015134894 and U.S. Pat. No. 9,079,949, the entire teachings of which are hereby incorporated by reference. The terms ravulizumab, BNJ441, and ALXN1210 may be used interchangeably throughout this document, but all refer to the same antibody. Ravulizumab selectively binds to human complement protein C5, inhibiting its cleavage to C5a and C5b during complement activation. This inhibition prevents the release of the proinflammatory mediator C5a and the formation of the cytolytic pore-forming membrane attack complex (MAC) C5b-9 while preserving the proximal or early components of complement activation (e.g., C3 and C3b) essential for the opsonization of microorganisms and clearance of immune complexes.

The polypeptide sequence of ravulizumab, as accessioned in KEGG DRUG Database (https://www.kegg.jp/entry/D11054), provides that the N-terminal amino acid of variable heavy chain is “X”, but the database does not state what X is. The Chemical Abstracts (CAS) for ravulizumab (CAS 1803171-55-2) also provides that N-terminal X is pyroglutamic acid (designated as “chain 1 pyroglutamic acid-1” in the CAS report). Although this information may seem different from the VH sequence of ravulizumab, e.g., a heavy chain variable region polypeptide comprising the amino acid sequence depicted in SEQ ID NO: 12 and/or a heavy chain polypeptide comprising the amino acid sequence depicted in SEQ ID NO: 14, there is alignment between patented sequences and the drug database/CAS sequence because it was recognized in the art that N-Terminal Q in polypeptide and/or antibody sequence cyclizes during process development to yield drug product conversion to pyroglutamate (Pryo-Q) near 100%, as disclosed in Liu et a!. (J Pharm Sci. 2019 October; 108(10):3194-3200) https://pubmed.ncbi.nlm.nih.gov/31145921/and Nguyen et al. (Int. J. Mol. Sci. 2017 Jul. 20; 18(7):1575) https://www.researchgate.net/figure/Cyclization-reactions-of-N-terminal-glutamine-and-glutamate-residues-in-a-polypeptide_fig4_318926365. Additional information is provided in page 7 and Table 4 of Xu et al. (MAbs, 2019 February/March; 11(2):239-264) and the following referenced publications: (1) Yu et al., “Investigation of N-terminal glutamatecyclization of recombinant monoclonal antibody in formulation development”, J. Pharm. Biomed. Anal., 2006, 42, 455-463 and Dick et al., “Determination of the origin of the N-terminal pyro-glutamatevariation in monoclonal antibodies using model peptides”, Biotechnol. Bioeng., 2007, 97, 544-553, the disclosures in which are incorporated by reference in their entirety.

In other embodiments, the antibody comprises the heavy and light chain CDRs or variable regions of ravulizumab. Accordingly, in one embodiment, the antibody comprises the CDR1, CDR2 and CDR3 domains of the VH region of ravulizumab having the sequence set forth in SEQ ID NO:12, and the CDR1, CDR2 and CDR3 domains of the VL region of ravulizumab having the sequence set forth in SEQ ID NO:8. In another embodiment, the antibody comprises heavy chain CDR1, CDR2 and CDR3 domains having the sequences set forth in SEQ ID NOs:19, 18 and 3, respectively, and light chain CDR1. CDR2 and CDR3 domains having the sequences set forth in SEQ ID NOs:4, 5 and 6, respectively. In another embodiment, the antibody comprises VH and VL regions having the amino acid sequences set forth in SEQ ID NO:12 and SEQ ID NO:8, respectively.

Another exemplary anti-C5 antibody is antibody BNJ421 comprising heavy and light chains having the sequences shown in SEQ ID NOs:20 and 11, respectively, or antigen binding fragments and variants thereof. BNJ421 (also known as ALXN1211) is described in WO2015134894 and U.S. Pat. No. 9,079,949, the entire teachings of which are hereby incorporated by reference.

In other embodiments, the antibody comprises the heavy and light chain CDRs or variable regions of BNJ421. Accordingly, in one embodiment, the antibody comprises the CDR1, CDR2 and CDR3 domains of the VH region of BNJ421 having the sequence set forth in SEQ ID NO: 12, and the CDR1, CDR2 and CDR3 domains of the VL region of BNJ421 having the sequence set forth in SEQ ID NO:8. In another embodiment, the antibody comprises heavy chain CDR1, CDR2 and CDR3 domains having the sequences set forth in SEQ ID NOs:19, 18 and 3, respectively, and light chain CDR1, CDR2 and CDR3 domains having the sequences set forth in SEQ ID NOs:4, 5 and 6, respectively. In another embodiment, the antibody comprises VH and VL regions having the amino acid sequences set forth in SEQ ID NO: 12 and SEQ ID NO:8, respectively.

The exact boundaries of CDRs are defined differently according to different methods. In some embodiments, the positions of the CDRs or framework regions within a light or heavy chain variable domain are as defined by Kabat et al. [(1991) “Sequences of Proteins of Immunological Interest.” NIH Publication No. 91-3242, U.S. Department of Health and Human Services. Bethesda, MD]. In such cases, the CDRs can be referred to as “Kabat CDRs” (e.g., “Kabat LCDR2” or “Kabat HCDR1”). In some embodiments, the positions of the CDRs of a light or heavy chain variable region are as defined by Chothia et al. (Nature, 342:877-83, 1989). Accordingly, these regions can be referred to as “Chothia CDRs” (e.g., “Chothia LCDR2” or “Chothia HCDR3”). In some embodiments, the positions of the CDRs of the light and heavy chain variable regions can be defined by a Kabat-Chothia combined definition. In such embodiments, these regions can be referred to as “combined Kabat-Chothia CDRs.” Thomas, C. et al. (Mol. Immunol., 33:1389-401, 1996) exemplifies the identification of CDR boundaries according to Kabat and Chothia numbering schemes.

Another exemplary anti-C5 antibody is the 7086 antibody described in U.S. Pat. Nos. 8,241,628 and 8,883,158. In one embodiment, the antibody comprises the heavy and light chain CDRs or variable regions of the 7086 antibody (see U.S. Pat. Nos. 8,241,628 and 8,883,158). In another embodiment, the antibody, or antigen binding fragment thereof, comprises heavy chain CDR1, CDR2 and CDR3 domains having the sequences set forth in SEQ ID NOs:21, 22 and 23, respectively, and light chain CDR1, CDR2 and CDR3 domains having the sequences set forth in SEQ ID NOs:24, 25 and 26, respectively. In another embodiment, the antibody, or antigen binding fragment thereof, comprises the VH region of the 7086 antibody having the sequence set forth in SEQ ID NO:27, and the VL region of the 7086 antibody having the sequence set forth in SEQ ID NO:28.

Another exemplary anti-C5 antibody is the 8110 antibody also described in U.S. Pat. Nos. 8,241,628 and 8,883,158. In one embodiment, the antibody comprises the heavy and light chain CDRs or variable regions of the 8110 antibody. In another embodiment, the antibody, or antigen binding fragment thereof, comprises heavy chain CDR1, CDR2 and CDR3 domains having the sequences set forth in SEQ ID NOs:29, 30 and 31, respectively, and light chain CDR1, CDR2 and CDR3 domains having the sequences set forth in SEQ ID NOs:32, 33 and 34, respectively. In another embodiment, the antibody comprises the VH region of the 8110 antibody having the sequence set forth in SEQ ID NO:35, and the VL region of the 8110 antibody having the sequence set forth in SEQ ID NO:36.

Another exemplary anti-C5 antibody is the 305LO5 antibody described in U.S. Pat. No. 9,765,135. In one embodiment, the antibody comprises the heavy and light chain CDRs or variable regions of the 305LO5 antibody. In another embodiment, the antibody, or antigen binding fragment thereof, comprises heavy chain CDR1, CDR2 and CDR3 domains having the sequences set forth in SEQ ID NOs:37, 38 and 39, respectively, and light chain CDR1. CDR2 and CDR3 domains having the sequences set forth in SEQ ID NOs:40, 41 and 42, respectively. In another embodiment, the antibody comprises the VH region of the 305LO5 antibody having the sequence set forth in SEQ ID NO:43, and the VL region of the 305LO5 antibody having the sequence set forth in SEQ ID NO:44.

Another exemplary anti-C5 antibody is the SKY59 antibody (Fukuzawa, T. et al., Sci. Rep., 7:1080, 2017). In one embodiment, the antibody comprises the heavy and light chain CDRs or variable regions of the SKY59 antibody. In another embodiment, the antibody, or antigen binding fragment thereof, comprises a heavy chain comprising SEQ ID NO:45 and a light chain comprising SEQ ID NO:46.

In some embodiments, the anti-C5 antibody comprises the heavy and light chain variable regions or heavy and light chains of the REGN3918 antibody (see U.S. Pat. No. 10,633,434). In some embodiments, the anti-C5 antibody, or antigen-binding fragment thereof, comprises a heavy chain variable region sequence set forth in SEQ ID NO: 47 and a light chain variable region comprising the sequence set forth in SEQ ID NO: 48. In some embodiments, the anti-C5 antibody, or antigen-binding fragment thereof, comprises a heavy chain sequence set forth in SEQ ID NO: 49 and a light chain sequence set forth in SEQ ID NO: 50.

In another embodiment, the anti-C5 antibody is a biosimilar of eculizumab (SOLIRIS®). For example, in one embodiment, the anti-C5 antibody is, for example, ABP 959 antibody (eculizumab biosimilar manufactured by Amgen Inc., USA), ELIZARIA® (eculizumab biosimilar manufactured by Generium JNC, Russia), SB12 (eculizumab biosimilar manufactured by Samsung Bioepis. Incheon, South Korea), ISU305 (eculizumab biosimilar from ISU Abxis, South Korea). ABLYZE® (eculizumab biosimilar from CinnaGen, Iran), BCD 148 (eculizumab biosimilar from Biocad Medical, Quebec, Canada), tesidolumab (manufactured by Novartis), Crovalimab (manufactured by Roche), CAN106 (manufactured by CanBridge Pharmaceuticals, China), or Pozelimab (manufactured by Regeneron).

In some embodiments, an anti-C5 antibody described herein comprises a heavy chain CDR1 comprising, or consisting of, the following amino acid sequence: GHIFSNYWIQ (SEQ ID NO:19). In some embodiments, an anti-C5 antibody described herein comprises a heavy chain CDR2 comprising, or consisting of, the following amino acid sequence: EILPGSGHTEYTENFKD (SEQ ID NO: 18). In some embodiments, an anti-C5 antibody described herein comprises a heavy chain variable region comprising the following amino acid sequence:

(SEQ ID NO: 12)
QVQLVQSGAE VKKPGASVKV SCKASGHIFS NYWIQWVRQA
PGQGLEWMGE ILPGSGHTEY TENFKDRVTM TRDTSTSTVY
MELSSLRSED TAVYYCARYF FGSSPNWYFD VWGQGTLVTV
SS.

In some embodiments, an anti-C5 antibody described herein comprises a light chain variable region comprising the following amino acid sequence:

(SEQ ID NO: 8)
DIQMTQSPSS LSASVGDRVT ITCGASENIY GALNWYQQKP
GKAPKLLIYG ATNLADGVPS RFSGSGSGTD FTLTISSLQP
EDFATYYCQN VLNTPLTFGQ GTKVEIK.

An anti-C5 antibody described herein can, in some embodiments, comprise a variant human Fc constant region that binds to human neonatal Fc receptor (FcRn) with greater affinity than that of the native human Fc constant region from which the variant human Fc constant region was derived. The Fc constant region can, for example, comprise one or more (e.g., two, three, four, five, six, seven, or eight or more) amino acid substitutions relative to the native human Fc constant region from which the variant human Fc constant region was derived. The substitutions can increase the binding affinity of an IgG antibody containing the variant Fc constant region to FcRn at pH 6.0, while maintaining the pH dependence of the interaction. Methods for testing whether one or more substitutions in the Fc constant region of an antibody increase the affinity of the Fc constant region for FcRn at pH 6.0 (while maintaining pH dependence of the interaction) are known in the art and exemplified in the working examples. See, e.g., WO2015134894 and U.S. Pat. No. 9,079,949 the disclosures of each of which are incorporated herein by reference in their entirety.

Substitutions that enhance the binding affinity of an antibody Fc constant region for FcRn are known in the art and include, e.g., (1) the M252Y/S254T/T256E triple substitution (Dall'Acqua, W. et al., J. Biol. Chem., 281:23514-24, 2006); (2) the M428L or T250Q/M428L substitutions (Hinton, P. et al., J. Biol. Chem., 279:6213-6, 2004; Hinton, P. et al., J. Immunol., 176:346-56, 2006); and (3) the N434A or T307/E380A/N434A substitutions (Petkova, S. et al., Int. Immunol., 18:1759-69, 2006). The additional substitution pairings: P257I/Q311I, P257I/N434H and D376V/N434H (Datta-Mannan, A. et al., J. Biol. Chem., 282:1709-17, 2007), the disclosures of each of which are incorporated herein by reference in their entirety.

In some embodiments, the variant constant region has a substitution at EU amino acid position 255 for valine. In some embodiments, the variant constant region has a substitution at EU amino acid position 309 for asparagine. In some embodiments, the variant constant region has a substitution at EU amino acid position 312 for isoleucine. In some embodiments, the variant constant region has a substitution at EU amino acid position 386.

In some embodiments, the variant Fc constant region comprises no more than 30 (e.g., no more than 29, 28, 27, 26, 25, 24, 23, 22, 21, 20, 19, 18, 17, 16, 15, 14, 13, 12, 11, 10, 9, 8, 7, 6, 5, 4, 3 or 2) amino acid substitutions, insertions, or deletions relative to the native constant region from which it was derived. In some embodiments, the variant Fc constant region comprises one or more amino acid substitutions selected from the group consisting of: M252Y, S254T, T256E, N434S, M428L, V2591, T250I and V308F. In some embodiments, the variant human Fc constant region comprises a methionine at position 428 and an asparagine at position 434 of a native human IgG Fc constant region, each in EU numbering.

In some embodiments, the variant Fc constant region comprises a 428L/434S double substitution as described in, e.g., U.S. Pat. No. 8,088,376.

In some embodiments the precise location of these mutations may be shifted from the native human Fc constant region position due to antibody engineering. For example, the 428L/434S double substitution when used in a IgG2/4 chimeric Fc may correspond to 429L and 435S as in the M429L and N435S variants found in ravulizumab and described in U.S. Pat. No. 9,079,949 the disclosure of which is incorporated herein by reference in its entirety.

In some embodiments, the variant constant region comprises a substitution at amino acid position 237, 238, 239, 248, 250, 252, 254, 255, 256, 257, 258, 265, 270, 286, 289, 297, 298, 303, 305, 307, 308, 309, 311, 312, 314, 315, 317, 325, 332, 334, 360, 376, 380, 382, 384, 385, 386, 387, 389, 424, 428, 433, 434 or 436 (EU numbering) relative to the native human Fc constant region. In some embodiments, the substitution is selected from the group consisting of: methionine for glycine at position 237; alanine for proline at position 238; lysine for serine at position 239; isoleucine for lysine at position 248; alanine, phenylalanine, isoleucine, methionine, glutamine, serine, valine, tryptophan, or tyrosine for threonine at position 250; phenylalanine, tryptophan, or tyrosine for methionine at position 252; threonine for serine at position 254; glutamic acid for arginine at position 255: aspartic acid, glutamic acid, or glutamine for threonine at position 256; alanine, glycine, isoleucine, leucine, methionine, asparagine, serine, threonine, or valine for proline at position 257: histidine for glutamic acid at position 258; alanine for aspartic acid at position 265; phenylalanine for aspartic acid at position 270; alanine, or glutamic acid for asparagine at position 286: histidine for threonine at position 289; alanine for asparagine at position 297; glycine for serine at position 298; alanine for valine at position 303; alanine for valine at position 305; alanine, aspartic acid, phenylalanine, glycine, histidine, isoleucine, lysine, leucine, methionine, asparagine, proline, glutamine, arginine, serine, valine, tryptophan, or tyrosine for threonine at position 307; alanine, phenylalanine, isoleucine, leucine, methionine, proline, glutamine, or threonine for valine at position 308; alanine, aspartic acid, glutamic acid, proline, or arginine for leucine or valine at position 309; alanine, histidine, or isoleucine for glutamine at position 311; alanine or histidine for aspartic acid at position 312; lysine or arginine for leucine at position 314; alanine or histidine for asparagine at position 315; alanine for lysine at position 317; glycine for asparagine at position 325: valine for isoleucine at position 332: leucine for lysine at position 334; histidine for lysine at position 360; alanine for aspartic acid at position 376; alanine for glutamic acid at position 380; alanine for glutamic acid at position 382; alanine for asparagine or serine at position 384; aspartic acid or histidine for glycine at position 385; proline for glutamine at position 386; glutamic acid for proline at position 387; alanine or serine for asparagine at position 389; alanine for serine at position 424; alanine, aspartic acid, phenylalanine, glycine, histidine, isoleucine, lysine, leucine, asparagine, proline, glutamine, serine, threonine, valine, tryptophan, or tyrosine for methionine at position 428; lysine for histidine at position 433; alanine, phenylalanine, histidine, serine, tryptophan, or tyrosine for asparagine at position 434; and histidine for tyrosine or phenylalanine at position 436, all in EU numbering.

Suitable anti-C5 antibodies for use in the methods described herein, in some embodiments, comprise a heavy chain polypeptide comprising the amino acid sequence set forth in SEQ ID NO:14 and/or a light chain polypeptide comprising the amino acid sequence set forth in SEQ ID NO: 11. Alternatively, the anti-C5 antibodies for use in the methods described herein, in some embodiments, comprise a heavy chain polypeptide comprising the amino acid sequence set forth in SEQ ID NO:20 and/or a light chain polypeptide comprising the amino acid sequence set forth in SEQ ID NO: 11.

In one embodiment, the antibody binds to C5 at pH 7.4 and 25 °C (and, otherwise, under physiologic conditions) with an affinity dissociation constant (K_{D}) that is at least 0.1 (e.g., at least 0.15, 0.175, 0.2, 0.25, 0.275, 0.3, 0.325, 0.35, 0.375, 0.4, 0.425, 0.45, 0.475, 0.5, 0.525, 0.55, 0.575, 0.6, 0.625, 0.65, 0.675, 0.7, 0.725, 0.75, 0.775, 0.8, 0.825, 0.85, 0.875, 0.9, 0.925, 0.95, or 0.975) nM. In one embodiment, the antibody binds to C5 at pH 7.4 and 25 °C (and, otherwise, under physiologic conditions) with an affinity dissociation constant (K_{D}) that is about 0.5 nM. In some embodiments, the K_{D} of the anti-C5 antibody, or antigen binding fragment thereof, is no greater than 1 (e.g., no greater than 0.9, 0.8, 0.7, 0.6, 0.5, 0.4, 0.3, or 0.2) nM. In some embodiments, the antibody binds to C5 at pH 6.0 and 25 °C (and, otherwise, under physiologic conditions) with a K_{D} that is about 22 nM.

In other embodiments, the [(K_{D} of the antibody for C5 at pH 6.0 at 25 °C)/(K_{D} of the antibody for C5 at pH 7.4 at 25 C)] is greater than 21 (e.g., greater than 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 350, 400, 450, 500, 600, 700, 800, 900, 1000, 1500, 2000, 2500, 3000, 3500, 4000, 4500, 5000, 5500, 6000, 6500, 7000, 7500 or 8000)

Methods for determining whether an antibody binds to a protein antigen and/or the affinity for an antibody to a protein antigen are known in the art. The binding of an antibody to a protein antigen, for example, can be detected and/or quantified using a variety of techniques such as, but not limited to, Western blot, dot blot, surface plasmon resonance (SPR) detection (e.g., BIAcore system; Pharmacia Biosensor AB, Uppsala, Sweden and Piscataway, N.J.), or enzyme-linked immunosorbent assay (ELISA; Benny K. C. Lo (2004) “Antibody Engineering: Methods and Protocols,” Humana Press (ISBN: 1588290921); Johne, B. et al., J. Immunol. Meth., 160:191-8, 1993; Jönsson, U. et al., Ann. Biol. Clin., 51:19-26, 1993; Jönsson. U. et al., Biotechniques, 11:620-7, 1991). In addition, methods for measuring the affinity (e.g., dissociation and association constants) are set forth in the working examples.

As used herein, the term “kₐ” refers to the rate constant for association of an antibody to an antigen. The term “k_{d}” refers to the rate constant for dissociation of an antibody from the antibody/antigen complex. And the term “K_{D}” refers to the equilibrium dissociation constant of an antibody-antigen interaction. The equilibrium dissociation constant is deduced from the ratio of the kinetic rate constants, K_{D}=kₐ/k_{d}. Such determinations can be measured, for example, at 25 °C or 37 °C. The kinetics of antibody binding to human C5 can be determined, for example, at pH 8.0, 7.4, 7.0, 6.5 and 6.0 via SPR on a BIAcore 3000 instrument using an anti-Fc capture method to immobilize the antibody.

In one embodiment, the anti-C5 antibody, or antigen binding fragment thereof, blocks the cleavage of C5 into C5a and C5b. Through this blocking effect, for example, the pro-inflammatory effects of C5a and the generation of the C5b-9 membrane attack complex (MAC) at the surface of a cell are inhibited.

Methods for determining whether a particular antibody described herein inhibits C5 cleavage are known in the art. Inhibition of human complement component C5 can reduce the cell-lysing ability of complement in a subject's body fluids. Such reductions of the cell-lysing ability of complement present in the body fluid(s) can be measured by methods known in the art such as, for example, by a conventional hemolytic assay such as the hemolysis assay (Kabat and Mayer (eds.), “Experimental Immunochemistry, 2^{nd} Edition,” 135-240, Springfield, IL, CC Thomas (1961), pages 135-139), or a conventional variation of that assay such as the chicken erythrocyte hemolysis method (Hillmen, P. et al., N. Engl. J. Med., 350:552-9, 2004). Methods for determining whether a candidate compound inhibits the cleavage of human C5 into forms C5a and C5b are known in the art (Evans, M. et al., Mol. Immunol., 32:1183-95, 1995). The concentration and/or physiologic activity of C5a and C5b in a body fluid can be measured, for example, by methods known in the art. For C5b, hemolytic assays or assays for soluble C5b-9 as discussed herein can be used. Other assays known in the art can also be used. Using assays of these or other suitable types, candidate agents capable of inhibiting human complement component C5 can be screened.

Immunological techniques such as, but not limited to, ELISA can be used to measure the protein concentration of C5 and/or its split products to determine the ability of an anti-C5 antibody, or antigen binding fragment thereof, to inhibit conversion of C5 into biologically active products. In some embodiments, C5a generation is measured. In some embodiments. C5b-9 neoepitope-specific antibodies are used to detect MAC formation.

Hemolytic assays can be used to determine the inhibitory activity of an anti-C5 antibody, or antigen binding fragment thereof, on complement activation. To determine the effect of an anti-C5 antibody, or antigen binding fragment thereof, on classical complement pathway-mediated hemolysis in a serum test solution in vitro, for example, sheep erythrocytes coated with hemolysin or chicken erythrocytes sensitized with anti-chicken erythrocyte antibody are used as target cells. The percentage of lysis is normalized by considering 100% lysis equal to the lysis occurring in the absence of the inhibitor. In some embodiments, the classical complement pathway is activated by a human IgM antibody, for example, as utilized in the Wieslab® Classical Pathway Complement Kit (Wieslab® COMPL CP310, Euro-Diagnostica, Sweden). Briefly, the test serum is incubated with an anti-C5 antibody, or antigen binding fragment thereof, in the presence of a human IgM antibody. The amount of C5b-9 that is generated is measured by contacting the mixture with an enzyme conjugated anti-C5b-9 antibody and a fluorogenic substrate and measuring the absorbance at the appropriate wavelength. As a control, the test serum is incubated in the absence of the anti-C5 antibody, or antigen binding fragment thereof. In some embodiments, the test serum is a C5-deficient serum reconstituted with a C5 polypeptide.

To determine the effect of an anti-C5 antibody, or antigen binding fragment thereof, on alternative pathway-mediated hemolysis, unsensitized rabbit or guinea pig erythrocytes can be used as the target cells. In some embodiments, the serum test solution is a C5-deficient serum reconstituted with a C5 polypeptide. The percentage of lysis is normalized by considering 100% lysis equal to the lysis occurring in the absence of the inhibitor. In some embodiments, the alternative complement pathway is activated by lipopolysaccharide molecules, for example, as utilized in the Wieslab® Alternative Pathway Complement Kit (Wieslab® COMPL AP330, Euro-Diagnostica, Sweden). Briefly, the test serum is incubated with an anti-C5 antibody, or antigen binding fragment thereof, in the presence of lipopolysaccharide. The amount of C5b-9 that is generated is measured by contacting the mixture with an enzyme conjugated anti-C5b-9 antibody and a fluorogenic substrate and measuring the fluorescence at the appropriate wavelength. As a control, the test serum is incubated in the absence of the anti-C5 antibody, or antigen binding fragment thereof.

In some embodiments, C5 activity, or inhibition thereof, is quantified using a CH50eq assay. The CH50eq assay is a method for measuring the total classical complement activity in serum. This test is a lytic assay, which uses antibody-sensitized erythrocytes as the activator of the classical complement pathway and various dilutions of the test serum to determine the amount required to give 50% lysis (CH50). The percent hemolysis can be determined, for example, using a spectrophotometer. The CH50eq assay provides an indirect measure of terminal complement complex (TCC) formation, since the TCC themselves are directly responsible for the hemolysis that is measured. The assay is known and commonly practiced by those of skill in the art. Briefly, to activate the classical complement pathway, undiluted serum samples (e.g., reconstituted human serum samples) are added to microassay wells containing the antibody-sensitized erythrocytes to thereby generate TCC. Next, the activated sera are diluted in microassay wells, which are coated with a capture reagent (e.g., an antibody that binds to one or more components of the TCC). The TCC present in the activated samples bind to the monoclonal antibodies coating the surface of the microassay wells. The wells are washed and to each well is added a detection reagent that is detectably labeled and recognizes the bound TCC. The detectable label can be, e.g., a fluorescent label or an enzymatic label. The assay results are expressed in CH50 unit equivalents per milliliter (CH50 U Eq/mL).

Inhibition, e.g., as it pertains to terminal complement activity, includes at least a 5 (e.g., at least a 6, 7, 8, 9, 10, 15, 20, 25, 30, 35, 40, 45, 50, 55 or 60) % decrease in the activity of terminal complement in, e.g., a hemolytic assay or CH50eq assay as compared to the effect of a control antibody (or antigen-binding fragment thereof) under similar conditions and at an equimolar concentration. Substantial inhibition, as used herein, refers to inhibition of a given activity (e.g., terminal complement activity) of at least 40 (e.g., at least 45, 50, 55, 60, 65, 70, 75, 80, 85, 90, or 95 or greater) %. In some embodiments, an anti-C5 antibody described herein contains one or more amino acid substitutions relative to the CDRs of eculizumab (i.e., SEQ ID NOs:1-6), yet retains at least 30 (e.g., at least 31, 32, 33, 34, 35, 36, 37, 38, 39, 40, 41, 42, 43, 44, 45, 46, 47, 48, 49, 50, 55, 60, 65, 70, 75, 80, 85, 90 or 95) % of the complement inhibitory activity of eculizumab in a hemolytic assay or CH50eq assay.

An anti-C5 antibody described herein has a serum half-life in humans that is at least 20 (e.g., at least 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54 or 55) days. In another embodiment, the anti-C5 antibody described herein has a serum half-life in humans that is at least 40 days. In another embodiment, the anti-C5 antibody described herein has a serum half-life in humans that is approximately 43 days. In another embodiment, the anti-C5 antibody described herein has a serum half-life in humans that is between 39-48 days. Methods for measuring the serum half-life of an antibody are known in the art. In some embodiments, an anti-C5 antibody, or antigen binding fragment thereof, described herein has a serum half-life that is at least 20 (e.g., at least 30, 35, 40, 45, 50, 55, 60, 65, 70, 75, 80, 85, 90, 95, 100, 125, 150, 175, 200, 250, 300, 400 or 500) % greater than the serum half-life of eculizumab, e.g., as measured in one of the mouse model systems described in the working examples (e.g., the C5-deficient/NOD/scid mouse or hFcRn transgenic mouse model system).

In one embodiment, the antibody competes for binding with, and/or binds to the same epitope on C5 as an antibody described herein. The term “binds to the same epitope” with reference to two or more antibodies means that the antibodies bind to the same segment of amino acid residues, as determined by a given method. Techniques for determining whether antibodies bind to the same epitope on C5 with an antibody described herein include, for example, epitope mapping methods, such as, x-ray analyses of crystals of antigen:antibody complexes, and hydrogen/deuterium exchange mass spectrometry (HDX-MS). Antibodies having the same VH and VL or the same CDR1, CDR2 and CDR3 sequences are expected to bind to the same epitope.

Antibodies that “compete with another antibody for binding to a target” refer to antibodies that inhibit (partially or completely) the binding of the other antibody to the target. Whether two antibodies compete with each other for binding to a target, i.e., whether and to what extent one antibody inhibits the binding of the other antibody to a target, may be determined using known competition experiments. In certain embodiments, an antibody competes with, and inhibits binding of another antibody to a target by at least 10%, 20%, 30%, 40%, 50%, 60%, 70%, 80%, 90% or 100%. The level of inhibition or competition may be different depending on which antibody is the “blocking antibody” (i.e., the antibody that is incubated first with the target). Competing antibodies can bind to, for example, the same epitope, an overlapping epitope or to adjacent epitopes (e.g., as evidenced by steric hindrance).

Anti-C5 antibodies, or antigen-binding fragments thereof described herein, used in the methods described herein can be generated using a variety of art-recognized techniques. Monoclonal antibodies can be obtained by various techniques familiar to those skilled in the art. Briefly, spleen cells from an animal immunized with a desired antigen are immortalized, commonly by fusion with a myeloma cell (Kohler, G. & Milstein, C., Eur. J. Immunol., 6:511-9, 1976)). Methods of immortalization include transformation with Epstein Barr Virus, oncogenes, or retroviruses or other methods known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences that encode a monoclonal antibody or a binding fragment thereof by screening a DNA library from human B cells (Huse, W. et al., Science, 246:1275-81, 1989).

### III. Compositions

Also provided herein are compositions comprising an anti-C5 antibody, or antigen binding fragment thereof. In one embodiment, the composition comprises an anti-C5 antibody, or antigen binding fragment thereof comprising the CDR1, CDR2 and CDR3 domains in a heavy chain variable region having the sequence set forth in SEQ ID NO:12, and the CDR1, CDR2 and CDR3 domains in a light chain variable region having the sequence set forth in SEQ ID NO:8. In another embodiment, the anti-C5 antibody comprises heavy and light chains having the sequences shown in SEQ ID NOs:14 and 11, respectively. In another embodiment, the anti-C5 antibody comprises heavy and light chains having the sequences shown in SEQ ID NOs:20 and 11, respectively.

The compositions can be formulated as a pharmaceutical solution, e.g., for administration to a subject according to any of the methods described herein. The pharmaceutical compositions generally include a pharmaceutically acceptable carrier. As used herein, a “pharmaceutically acceptable carrier” refers to, and includes, any and all solvents, dispersion media, coatings, antibacterial and antifungal agents, isotonic and absorption delaying agents, and the like that are physiologically compatible. The compositions can include a pharmaceutically acceptable salt, e.g., an acid addition salt or a base addition salt, sugars, carbohydrates, polyols and/or tonicity modifiers.

The compositions can be formulated according to standard methods. Pharmaceutical formulation is an established art (see, for example, Gennaro (2000) “Remington: The Science and Practice of Pharmacy,” 20^{th} Edition, Lippincott, Williams & Wilkins (ISBN: 0683306472); Ansel et al. (1999) “Pharmaceutical Dosage Forms and Drug Delivery Systems,” 7^{th} Edition, Lippincott Williams & Wilkins Publishers (ISBN: 0683305727); and Kibbe (2000) “Handbook of Pharmaceutical Excipients American Pharmaceutical Association,” 3^{rd} Edition (ISBN: 091733096X)). In some embodiments, a composition can be formulated, for example, as a buffered solution at a suitable concentration and suitable for storage at 2-8 C (e.g., 4 C). In some embodiments, a composition can be formulated for storage at a temperature below 0 C (e.g., −20 C or −80 C). In some embodiments, the composition can be formulated for storage for up to 2 years (e.g. 1 month, 2 months, 3 months, 4 months, 5 months, 6 months, 7 months, 8 months, 9 months, 10 months, 11 months, 1 year, 11% years or 2 years) at 2-8 C (e.g., 4 C). Thus, in some embodiments, the compositions described herein are stable in storage for at least 1 year at 2-8 C (e.g., 4 C).

The pharmaceutical compositions can be in a variety of forms. These forms include. e.g., liquid, semi-solid and solid dosage forms, such as liquid solutions (e.g., injectable and infusible solutions), dispersions or suspensions, tablets, pills, powders, liposomes and suppositories. The preferred form depends, in part, on the intended mode of administration and therapeutic application. Compositions containing a composition intended for systemic or local delivery, for example, can be in the form of injectable or infusible solutions. Accordingly, the compositions can be formulated for administration by a parenteral mode (e.g., intravenous, subcutaneous, intraperitoneal, or intramuscular injection). “Parenteral administration.” “administered parenterally” and other grammatically equivalent phrases, as used herein, refer to modes of administration other than enteral and topical administration, usually by injection, and include, without limitation, intravenous, intranasal, intraocular, pulmonary, intramuscular, intraarterial, intrathecal, intracapsular, intraorbital, intracardiac, intradermal, intrapulmonary, intraperitoneal, transtracheal, subcutaneous, subcuticular, intraarticular, subcapsular, subarachnoid, intraspinal, epidural, intracerebral, intracranial, intracarotid and intrasternal injection and infusion.

### IV. Methods

Provided herein are methods involving preparing a human patient for surgery (e.g., cardiac surgery with cardiopulmonary bypass (CPB)) in a particular sub-population (e.g., a human patient with kidney disease, including chronic kidney disease (CKD)), methods of inhibiting terminal complement activation in a human patient, methods of treating a human patient with CKD prior to cardiac surgery with CPB, methods of preventing or reducing (e.g., minimizing) cardiac surgery associated acute kidney injury (CSA-AKI) in a human patient with CKD, and methods of preventing or reducing (e.g., minimizing) one or more MAKE in a human patient with CKD.

In one embodiment, the dose of the anti-C5 antibody, or antigen binding fragment thereof, is based on the weight of the patient. For example, in one embodiment, a 2700 mg dose of the anti-C5 antibody, or antigen binding fragment thereof, is administered to a patient weighing ≥30 to <40 kg. In another embodiment, a 3000 mg dose of the anti-C5 antibody, or antigen binding fragment thereof, is administered to a patient weighing ≥40 to <60 kg. In another embodiment, a 3300 mg dose of the anti-C5 antibody, or antigen binding fragment thereof, is administered to a patient weighing ≥60 to <100 kg. In another embodiment, a 3600 mg dose of the anti-C5 antibody, or antigen binding fragment thereof, is administered to a patient weighing ≥100 kg. In certain embodiments, dosage regimens are adjusted to provide the optimum desired response (e.g., an effective response).

In one aspect, a method of preparing a human patient (e.g., a human patient with kidney disease, including CKD) for surgery (e.g., cardiac surgery with CPB) is provided, wherein the method comprises administering to the patient an effective amount of an anti C5 antibody, or antigen binding fragment thereof, wherein the anti C5 antibody, or antigen binding fragment thereof, is administered once prior to the surgery.

In another embodiment, a method of preparing a human patient (e.g., a human patient with kidney disease, including CKD) for surgery (e.g., cardiac surgery with CPB) is provided, wherein the method comprises administering to the patient an effective amount of an anti C5 antibody, or antigen binding fragment thereof, comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, wherein the anti C5 antibody, or antigen binding fragment thereof, is administered once prior to the surgery.

In another embodiment, a method of preparing a human patient with CKD for cardiac surgery with CPB is provided, wherein the method comprises administering to the patient an effective amount of an anti C5 antibody, or antigen binding fragment thereof, comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, wherein the anti C5 antibody, or antigen binding fragment thereof, is administered prior to the surgery once at a dose of:
e) 2700 mg to a patient weighing ≥30 to <40 kg;
f) 3000 mg to a patient weighing ≥40 to <60 kg;
g) 3300 mg to a patient weighing ≥60 to <100 kg; or
h) 3600 mg to a patient weighing ≥100 kg.

In another aspect, a method of inhibiting terminal complement activation in a human patient (e.g., a human patient with kidney disease, including CKD) prior to surgery (e.g., cardiac surgery with CPB) is provided, wherein the method comprises administering to the patient an effective amount of an anti C5 antibody, or antigen binding fragment thereof, wherein the anti C5 antibody, or antigen binding fragment thereof, is administered once prior to the surgery.

In one embodiment, a method of inhibiting terminal complement activation in a human patient (e.g., a human patient with kidney disease, including CKD) prior to surgery (e.g., cardiac surgery with CPB) is provided, wherein the method comprises administering to the patient an effective amount of an anti C5 antibody, or antigen binding fragment thereof, comprising CDR1. CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, wherein the anti C5 antibody, or antigen binding fragment thereof, is administered once prior to the surgery.

In another embodiment, a method of inhibiting terminal complement activation in a human patient with CKD prior to cardiac surgery with CPB is provided, wherein the method comprises administering to the patient an effective amount of an anti C5 antibody, or antigen binding fragment thereof, comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, wherein the anti C5 antibody, or antigen binding fragment thereof, is administered prior to the surgery once at a dose of:
a) 2700 mg to a patient weighing ≥30 to <40 kg;
b) 3000 mg to a patient weighing ≥40 to <60 kg;
c) 3300 mg to a patient weighing ≥60 to <100 kg; or
d) 3600 mg to a patient weighing ≥100 kg.

In some embodiments, terminal complement activation is inhibited in a human patient, according to the methods described herein, as assessed by any suitable assay. In one embodiment, the method inhibits terminal complement activation in a human patient, for example, by 50%, 55%, 60%, 65%, 70%, 75%, 80%, 85%, 86%, 87%, 88%, 89%, 90%, 91%, 92%, 93%, 94%, 95%, 96%, 97%, 98%, 99%, or 100%.

In another aspect, a method of treating a human patient with kidney disease (e.g., CKD) prior to cardiac surgery (e.g., with CPB), is provided, wherein the method comprises administering to the patient an effective amount of an anti-C5 antibody, or antigen binding fragment thereof, wherein the anti-C5 antibody, or antigen binding fragment thereof, is administered once prior to the surgery.

In one embodiment, a method of treating a human patient with kidney disease (e.g., CKD) prior to cardiac surgery (e.g., with CPB), is provided, wherein the method comprises administering to the patient an effective amount of an anti-C5 antibody, or antigen binding fragment thereof, comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, wherein the anti-C5 antibody, or antigen binding fragment thereof, is administered once prior to the surgery.

In another embodiment, a method of treating a human patient with CKD prior to cardiac surgery with CPB, is provided, wherein the method comprises administering to the patient an effective amount of an anti-C5 antibody, or antigen binding fragment thereof, comprising CDR1, CDR2 and CDR3 heavy chain sequences as set forth in SEQ ID NOs:19, 18 and 3, respectively, and CDR1. CDR2 and CDR3 light chain sequences as set forth in SEQ ID NOs:4, 5 and 6, respectively, wherein the anti-C5 antibody, or antigen binding fragment thereof, is administered prior to the surgery once at a dose of:
a) 2700 mg to a patient weighing ≥30 to <40 kg;
b) 3000 mg to a patient weighing ≥40 to <60 kg;
c) 3300 mg to a patient weighing ≥60 to <100 kg; or
d) 3600 mg to a patient weighing ≥100 kg.

In another aspect, a method of preventing or reducing CSA-AKI in a human patient with kidney disease (e.g., CKD) is provided, wherein the method comprises administering to the patient an effective amount of an anti-C5 antibody, or antigen binding fragment thereof, and wherein the anti-C5 antibody, or antigen binding fragment thereof, is administered once prior to cardiac surgery (e.g., with CPB).

In one embodiment, a method of preventing or reducing CSA-AKI in a human patient with kidney disease (e.g., CKD) is provided, wherein the method comprises administering to the patient an effective amount of an anti-C5 antibody, or antigen binding fragment thereof, wherein the anti-C5 antibody, or antigen binding fragment thereof, comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, and wherein the anti-C5 antibody, or antigen binding fragment thereof, is administered once prior to cardiac surgery (e.g., with CPB).

In another embodiment, a method of preventing or reducing CSA-AKI in a human patient with CKD is provided, wherein the method comprises administering to the patient an effective amount of an anti-C5 antibody, or antigen binding fragment thereof, wherein the anti-C5 antibody, or antigen binding fragment thereof, comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, and wherein the anti-C5 antibody, or antigen binding fragment thereof, is administered prior to cardiac surgery (e.g., with CPB) once at a dose of:
a) 2700 mg to a patient weighing ≥30 to <40 kg;
b) 3000 mg to a patient weighing ≥40 to <60 kg;
c) 3300 mg to a patient weighing ≥60 to <100 kg; or
d) 3600 mg to a patient weighing ≥100 kg.

In another aspect, a method of preventing or reducing one or more MAKE in a human patient with kidney disease (e.g., CKD) is provided, wherein the method comprises administering to the patient an effective amount of an anti-C5 antibody, or antigen binding fragment thereof, wherein the anti-C5 antibody, or antigen binding fragment thereof, is administered once prior to cardiac surgery (e.g., with CPB).

In one embodiment, a method of preventing or reducing one or more MAKE in a human patient with kidney disease (e.g., CKD) is provided, wherein the method comprises administering to the patient an effective amount of an anti-C5 antibody, or antigen binding fragment thereof, wherein the anti-C5 antibody, or antigen binding fragment thereof, comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, and wherein the anti-C5 antibody, or antigen binding fragment thereof, is administered once prior to cardiac surgery (e.g., with CPB).

In another embodiment, a method of preventing or reducing one or more MAKE in a human patient with CKD is provided, wherein the method comprises administering to the patient an effective amount of an anti-C5 antibody, or antigen binding fragment thereof, wherein the anti-C5 antibody, or antigen binding fragment thereof, comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, and wherein the anti-C5 antibody, or antigen binding fragment thereof, is administered prior to cardiac surgery (e.g., with CPB), once at a dose of:
a) 2700 mg to a patient weighing ≥30 to <40 kg;
b) 3000 mg to a patient weighing ≥40 to <60 kg;
c) 3300 mg to a patient weighing ≥60 to <100 kg; or
d) 3600 mg to a patient weighing ≥100 kg.

In one embodiment, the anti-C5 antibody, or antigen binding fragment thereof, is administered to the patient at least one calendar day prior to the surgery (e.g., cardiac surgery with CPB). In another embodiment the anti-C5 antibody, or antigen binding fragment thereof, is administered from one to seven calendar days prior to the surgery. For example, in one embodiment, the anti-C5 antibody, or antigen binding fragment thereof, is administered one, two, three, four, five, six, or seven calendar days prior to the surgery.

### V. Outcomes

In certain embodiments, the methods described herein provide an optimal desired response (e.g., inhibits terminal complement activation in a human patient (e.g., a human patient with kidney disease, including CKD) prior to surgery (e.g., cardiac surgery with CPB), prevents or reduces CSA-AKI in a human patient with kidney disease (e.g., CKD) having surgery (e.g., cardiac surgery with CPB), and/or prevents or reduces one or more MAKE in a human patient with kidney disease (e.g., CKD) having surgery (e.g., cardiac surgery with CPB)).

In certain embodiments, the methods described herein are sufficient to maintain particular serum trough concentrations of the anti-C5 antibody, or antigen binding fragment thereof. In one embodiment, for example, the method maintains a serum trough concentration of the anti-C5 antibody, or antigen binding fragment thereof, of 50, 55, 60, 65, 70, 75, 80, 85, 90, 95, 100, 105, 110, 115, 120, 125, 130, 135, 140, 145, 150, 155, 160, 165, 170, 175, 180, 185, 190, 200, 205, 210, 215, 220, 225, 230, 240, 245, 250, 255, 260, 265, 270, 280, 290, 300, 305, 310, 315, 320, 325, 330, 335, 340, 345, 350, 355, 360, 365, 370, 375, 380, 385, 390, 395, 400, 405, 410, 415, 420, 425, 430.435, 440, 445, 450, 455.460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700 μg/mL or greater. In one embodiment, the method maintains a serum trough concentration of the anti-C5 antibody, or antigen binding fragment thereof, of 100 μg/mL or greater, 150 μg/mL or greater, 200 μg/mL or greater, 250 μg/mL or greater, 300 μg/mL or greater, 350 μg/mL or greater, 400 μg/mL or greater, or 450 μg/mL or greater.

In another embodiment, the method maintains a serum trough concentration of the anti-C5 antibody, or antigen binding fragment thereof, of between 100 μg/mL and 700 μg/mL, preferably between 300 μg/mL and 600 μg/mL. In another embodiment, the method maintains a serum trough concentration of the anti-C5 antibody, or antigen binding fragment thereof, of about 475 μg/mL. In another embodiment, the method maintains a serum peak concentration of the anti-C5 antibody, or antigen binding fragment thereof, of less than about 1800, 1780, 1760, 1740, 1720, 1700, 1680, 1660, 1640, 1620, 1600, 1580, 1560, 1540, 1520, 1500, 1480, 1460, 1440, 1420, 1400, 1380, 1360, 1340, 1320, 1300, 1280, 1260, 1240, 1220, 1200, 1180, 1160, 1140, 1120, 1100, 1080, 1060, 1040, 1020, 1000, 980, 960, 940, 920, or 900 μg/mL, or less. In other embodiments, the method maintains a peak serum concentration of the anti-C5 antibody, or antigen binding fragment thereof, of between 900 μg/mL and 1800 μg/mL, preferably between 1050 μg/mL and 1550 μg/mL. In another embodiment, the method maintains a peak serum concentration of the anti-C5 antibody, or antigen binding fragment thereof, of about 1350 μg/mL.

In another embodiment, the anti-C5 antibody, or antigen binding fragment thereof, is administered to the patient in an amount and with a frequency to maintain at least 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg, 200 μg, 205 μg, 210 μg, 215 μg, 220 μg, 225 μg, 230 μg, 235 μg, 240 μg, 245 μg, 250 μg, 255 μg, 260 μg, 270 μg, 280 μg, 290 μg, 300 μg, 320 μg, 340 μg, 360 μg, 380 μg, 400 μg, 420 μg, 440 μg, 460 μg, 480 μg, 500 μg, 550 μg, 600 μg, 650 μg, 700 μg, 750 μg, 800 μg, 850 μg, 900 μg, 950 μg, 1000 μg, 1050 μg, 1100 μg, 1150 μg, 1200 μg, 1250 μg, 1300 μg, 1350 μg, 1400 μg, 1450 μg, 1500 μg, 1550 μg, 1600 μg, 1650 μg, 1700 μg, 1750 μg, or more, e.g., 1800 μg of antibody per milliliter of the patient's blood.

In another embodiment, anti-C5 antibody, or antigen binding fragment thereof, is administered to the patient in an amount and with a frequency to maintain a minimum free C5 concentration. In one embodiment, for example, the anti-C5 antibody, or antigen binding fragment thereof, is administered to the patient in an amount and with a frequency to maintain a free C5 concentration of 0.5 μg/mL or less (e.g., 0.4 μg/mL, 0.3 μg/mL, 0.2 μg/mL, or 0.1 μg/mL or less).

The efficacy of the methods provided herein can be assessed using any suitable means. In one embodiment, the single pre-operative weight-based dose of the anti-C5 antibody, or antigen binding fragment thereof, results in at least 18 days of complete C5 inhibition.

In one embodiment, the method inhibits terminal complement activation in a human patient, as assessed by any suitable assay. In one embodiment, the method inhibits terminal complement activation in a human patient, for example, by 50%, 55%, 60%, 65%, 70%, 75%, 80%, 85%, 86%, 87%, 88%, 89%, 90%, 91%, 92%, 93%, 94%, 95%, 96%, 97%, 98%, 99%, or 100%.

In another embodiment, the methods described herein prevent the need for kidney replacement therapy (KRT).

In another embodiment, the method prevents or reduces CSA-AKI in the human patient with CKD. In one embodiment, CSA-AKI is characterized by an increase in:
a) serum creatinine (sCr) or serum Cystatin C (sCysC) by ≥0.3 mg/dL in a 48-hour period within 7 days following CPB and/or
b) sCr or or sCysC≥1.5 times baseline within 7 days following CPB or at Day 15, 30, 60 or 90 post CPB.

In another embodiment, the human patient is free of severe CSA-AKI (Stage 2 or 3) based on highest observed sCr within 7, 30, 45, 60, or 90 days post CPB, as assessed by modified Kidney Disease Improving Global Outcomes (KDIGO) criteria, as set forth in Table 1 in the Example (see, also KDIGO., Kidney Inter. Suppl. 2013; 3:1-150 and Khwaja A., Nephron. Clin. Pract. 2012; 120(4):c179-184).

In another embodiment, the human patient is free of severe CSA-AKI based on highest observed sCr, within 7, 30, 45, 60, or 90 days post CPB, as assessed by Modified “Risk, Injury, Failure, Loss of kidney function, and End-stage kidney disease” (RIFLE) Criteria, as set forth in Table 2 in the Example (see, also Bellomo R, et al., Acute Dialysis Quality Initiative workgroup. Acute renal failure—definition, outcome measures, animal models, fluid therapy and information technology needs: the Second International Consensus Conference of the Acute Dialysis Quality Initiative (ADQI) Group. Crit Care. 2004; 8(4):R204-R212).

In another embodiment, the method results in an improvement in the stage of Post-CSA-AKI Kidney Function, as set forth in Table 3 in the Example (see also Chawla L S, et al., Acute kidney disease and renal recovery: consensus report of the Acute Disease Quality Initiative (ADQI) 16 Workgroup. Nat Rev Nephrol. 2017; 13(4):241-257).

In another embodiment, the method results in stable CSA-AKI within 7, 30, 45, 60, or 90 days post-surgery, characterized by sCr≥2.0−<3.0×baseline.

In another embodiment, the method results in an improvement from CSA-AKI within 7, 30, 45, 60, or 90 days post-surgery, wherein the improvement is characterized by sCr≥1.5−<2.0×baseline.

In another embodiment, the method results in a partial recovery from CSA-AKI within 7, 30, 45, 60, or 90 days post-surgery, wherein the partial recovery is characterized by sCr≥1.1−<1.5×baseline.

In another embodiment, the method results in a complete recovery from CSA-AKI within 7, 30, 45, 60, or 90 days post-surgery, wherein the complete recovery is characterized by sCr<1.1×baseline.

In another embodiment, the method prevents or reduces one or more MAKE in the human patient with CKD. In one embodiment, the one or more MAKE is sustained kidney dysfunction (SKD) defined as an estimated glomerular filtration rate (eGFR)>25% below baseline post CPB, for example, wherein the decrease in eGFR is determined by the Chronic Kidney Disease Epidemiology Collaboration (CKD-EPI) formula based on serum Cystatin C (sCysC) or serum creatinine (sCr). In another embodiment, the one or more MAKE is the occurrence of kidney replacement therapy (KRT) post CPB. In another embodiment, the one or more MAKE is death from any cause post CPB.

In another embodiment, the method results in a change from baseline in quality of life as assessed via a Quality of Life Assessment. For example, in one embodiment, the Quality of Life Assessment is a Kidney Disease Quality of Life instrument (KDQOL-36™) The KDQOL-36™ is a 36-item kidney-specific health-related quality of life measure, including Short-Form Health Survey 12 Item (SF-12) as generic core plus the burden of kidney disease, symptoms/problems of kidney disease, and effects of kidney disease scales.

In another embodiment, the Quality of Life Assessment is The European Quality of Life Group's 5 dimension 5-level (EQ-5D-5L). The EQ-5D-5L is a self-assessed, standardized instrument to measure health related quality of life and has been used in a wide range of health conditions. The EQ 5D 5L comprises 5 dimensions, each describing a different aspect of health: mobility, self-care, usual activities, pain/discomfort, and anxiety/depression.

In another embodiment, the Quality of Life Assessment is The Functional Assessment of Chronic Illness Therapy (FACIT) Fatigue scale. The FACIT scale is a 13-item questionnaire that assesses self-reported fatigue and its impact upon daily activities and function over the preceding 7 days.

In another embodiment, the method results in a shift toward normal levels of biomarkers associated with vascular inflammation (e.g., shed tumor necrosis factor receptor 1 [TNF-R1]). In another embodiment, the method results in a shift toward normal levels of biomarkers associated with endothelial damage and/or activation (e.g., thrombomodulin). In another embodiment, the method results in a shift toward normal levels of biomarkers associated with renal injury (e.g., neutrophil gelatinase-associated lipocalin [NGAL]). In another embodiment, the method results in a shift toward normal levels of biomarkers associated with inducers of cell-cycle arrest (e.g., tissue inhibitor of metalloproteinase-2 [TIMP-2]). In another embodiment, the method results in a shift toward normal levels of complement proteins and complement activation pathway products (e.g., soluble C5b-9).

### VI. Kits and Unit Dosage Forms

Also provided herein are kits that include a pharmaceutical composition containing an anti-C5 antibody, or antigen binding fragment thereof, such as ravulizumab, and a pharmaceutically acceptable carrier, in a therapeutically effective amount adapted for use in the preceding methods. The kits optionally also can include instructions, e.g., comprising administration schedules, to allow a practitioner (e.g., a physician, nurse, or patient) to administer the composition contained therein to administer the composition to a patient. The kit also can include a syringe.

Optionally, the kits include multiple packages of the single-dose pharmaceutical compositions each containing an effective amount of the anti-C5 antibody, or antigen binding fragment thereof, for a single administration in accordance with the methods provided above. Instruments or devices necessary for administering the pharmaceutical composition(s) also may be included in the kits. For instance, a kit may provide one or more pre-filled syringes containing an amount of the anti-C5 antibody, or antigen binding fragment thereof.

In one embodiment, a kit comprises: (a) a dose of an anti-C5 antibody, or antigen binding fragment thereof, CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively and (b) instructions for using the anti-C5 antibody, or antigen binding fragment thereof, in any of the methods described herein.

### VI. Uses

In another aspect, an anti-C5 antibody, or antigen-binding fragment thereof, (e.g., ravulizumab (ULTOMIRIS®)), for use in preparing a human patient with CKD for cardiac surgery with CPB is provided, wherein the anti-C5 antibody, or antigen binding fragment thereof, is administered prior to the surgery once at a dose of:
a) 2700 mg to a patient weighing 30 to <40 kg;
b) 3000 mg to a patient weighing 40 to <60 kg;
c) 3300 mg to a patient weighing 60 to <100 kg; or
d) 3600 mg to a patient weighing 100 kg.

In another embodiment, an anti-C5 antibody, or antigen-binding fragment thereof, for use in inhibiting terminal complement activation in a human patient with CKD prior to cardiac surgery with CPB is provided, wherein the anti-C5 antibody, or antigen binding fragment thereof, is administered prior to the surgery once at a dose of:
a) 2700 mg to a patient weighing 30 to <40 kg;
b) 3000 mg to a patient weighing 40 to <60 kg;
c) 3300 mg to a patient weighing 60 to <100 kg; or
d) 3600 mg to a patient weighing 100 kg.

In another embodiment, an anti-C5 antibody, or antigen-binding fragment thereof, for use in treatment of a human patient with CKD prior to cardiac surgery with CPB is provided, wherein the anti-C5 antibody, or antigen binding fragment thereof, is administered prior to the surgery once at a dose of:
a) 2700 mg to a patient weighing 30 to <40 kg;
b) 3000 mg to a patient weighing 40 to <60 kg;
c) 3300 mg to a patient weighing ≥60 to <100 kg; or
d) 3600 mg to a patient weighing ≥100 kg.

In another embodiment, an anti-C5 antibody, or antigen-binding fragment thereof, for use in preventing or reducing CSA-AKI in a human patient with CKD is provided, wherein the anti-C5 antibody, or antigen binding fragment thereof, is administered prior to the surgery once at a dose of:
a) 2700 mg to a patient weighing ≥30 to <40 kg;
b) 3000 mg to a patient weighing ≥40 to <60 kg;
c) 3300 mg to a patient weighing ≥60 to <100 kg; or
d) 3600 mg to a patient weighing ≥100 kg.

In another embodiment, an anti-C5 antibody, or antigen-binding fragment thereof, for use in preventing or reducing one or more MAKEs in a human patient with CKD is provided, wherein the anti-C5 antibody, or antigen binding fragment thereof, is administered prior to the surgery once at a dose of:
a) 2700 mg to a patient weighing ≥30 to <40 kg;
b) 3000 mg to a patient weighing ≥40 to <60 kg;
c) 3300 mg to a patient weighing ≥60 to <100 kg; or
d) 3600 mg to a patient weighing ≥100 kg.

In a further aspect, the invention provides for the use of an anti-C5, antibody or antigen-binding fragment thereof (e.g., ravulizumab (ULTOMIRIS®)), for preparing a human patient with CKD for cardiac surgery with CPB, wherein the anti-C5 antibody, or antigen binding fragment thereof, is administered prior to the surgery once at a dose of:
a) 2700 mg to a patient weighing ≥30 to <40 kg;
b) 3000 mg to a patient weighing ≥40 to <60 kg;
c) 3300 mg to a patient weighing ≥60 to <100 kg; or
d) 3600 mg to a patient weighing ≥100 kg.

In one embodiment, the use of an anti-C5, antibody or antigen-binding fragment thereof, for inhibiting terminal complement activation in a human patient with CKD prior to cardiac surgery with CPB is provided, wherein the anti C5 antibody, or antigen binding fragment thereof, is administered prior to the surgery once at a dose of:
a) 2700 mg to a patient weighing ≥30 to <40 kg;
b) 3000 mg to a patient weighing ≥40 to <60 kg;
c) 3300 mg to a patient weighing ≥60 to <100 kg; or
d) 3600 mg to a patient weighing ≥100 kg.

In another embodiment, the use of an anti-C5, antibody or antigen-binding fragment thereof, for treatment of a human patient with CKD prior to cardiac surgery with CPB is provided, wherein the anti-C5 antibody, or antigen binding fragment thereof, is administered prior to the surgery once at a dose of:
a) 2700 mg to a patient weighing ≥30 to <40 kg;
b) 3000 mg to a patient weighing ≥40 to <60 kg;
c) 3300 mg to a patient weighing ≥60 to <100 kg; or
d) 3600 mg to a patient weighing ≥100 kg.

In another embodiment, the use of an anti-C5 antibody, or antigen-binding fragment thereof, in preventing or reducing CSA-AKI in a human patient with CKD is provided, wherein the anti-C5 antibody, or antigen binding fragment thereof, is administered prior to the surgery once at a dose of:
a) 2700 mg to a patient weighing ≥30 to <40 kg;
b) 3000 mg to a patient weighing ≥40 to <60 kg;
c) 3300 mg to a patient weighing ≥60 to <100 kg; or
d) 3600 mg to a patient weighing ≥100 kg.

In another embodiment, the use of an anti-C5 antibody, or antigen-binding fragment thereof, in preventing or reducing one or more MAKEs in a human patient with chronic kidney disease, wherein the anti-C5 antibody, or antigen binding fragment thereof, is administered prior to the surgery once at a dose of:
a) 2700 mg to a patient weighing ≥30 to <40 kg;
b) 3000 mg to a patient weighing ≥40 to <60 kg;
c) 3300 mg to a patient weighing ≥60 to <100 kg; or
d) 3600 mg to a patient weighing ≥100 kg.

The following example is merely illustrative and should not be construed as limiting the scope of this disclosure in any way as many variations and equivalents will become apparent to those skilled in the art upon reading the present disclosure. The contents of all references, Genbank entries, patents and published patent applications cited throughout this application are expressly incorporated herein by reference.

### EXAMPLE

### Example: a Phase 3 Study of Ravulizumab (ULTOMIRIS®) to Protect Patients with Chronic Kidney Disease (CKD) from Cardiac Surgery Associated Kidney Injury (CSA-AKI) and or Subsequent Major Adverse Kidney Events (MAKE)

Patients with CKD undergoing cardiac surgery with CPB are at high risk for CSA-AKI. While CSA-AKI in the broad population having cardiac surgery with CPB occurs in 20-25% of patients, in the setting of CKD occurs in 60-80% of patients with MAKE occurring in 20-30% of patients. Inhibiting terminal complement from injuring the kidney and vasculature could reduce the incidence, severity, and duration of post-operative CSA-AKI and reduce subsequent MAKE, improving long-term survival and freedom from kidney replacement therapy (KRT), and reducing progression of CKD.

Accordingly, a randomized, placebo-controlled, double-blind study design is conducted to minimize bias and to balance the effect of confounding factors in this complex and highly comorbid population. Participants in both treatment groups receive standard of care as background therapy throughout the study.

### 1. Study Design

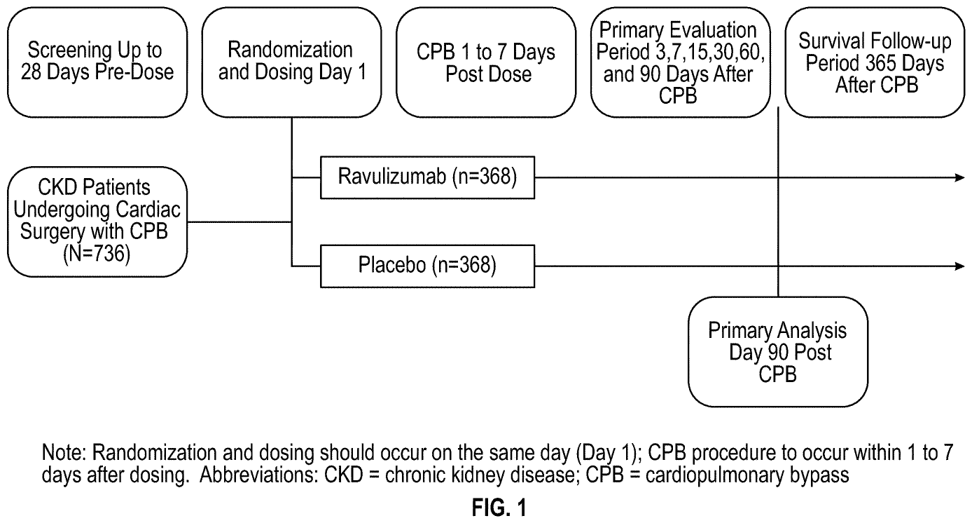

This is a Phase 3, randomized, double-blind, placebo-controlled, multicenter study of ravulizumab in adult participants with CKD and stable cardiac disease undergoing non-emergent sternotomy with CPB for coronary artery bypass graft (CABG), valve replacement or repair, or combined procedure, to reduce the risk of post-operative AKI and subsequent MAKE at 90 days (MAKE90) after surgery. A schematic of the trial design is set forth in FIG. 1. Participants considered at risk for AKI after CPB have eGFR≥20 to <60 mL/min/1.73 m2 with minimum Society of Thoracic Surgeons (STS) Calculator Renal Failure Risk Score of 3%.

The study consists of a Screening Period of up to 28 days with Randomization and Dosing within 1 to 7 days prior to surgery with CPB, a 90-day Primary Evaluation Period post CPB, and a Survival Follow-up Period 365 days post CPB.

Approximately 736 participants are randomized in a 1:1 ratio to treatment with either ravulizumab or placebo. Randomization is stratified by baseline CKD stage (3A, 3B, 4) and surgery type (mitral valve replacement or combined procedures vs other single procedure).

Eligible participants are randomized and receive a single weight-based dose of ravulizumab or placebo. Randomization and dosing occurs on the same day, except in cases when study intervention needs to be prepared the day before dosing. Dosing must occur at least 1 day prior to surgery and surgery must occur within 1 to 7 days after dosing. The date of the dosing is Day 1 for each participant. All treated participants are followed for 90 days after CPB procedure during the Primary Evaluation Period and a survival follow-up is completed on Day 365 post CPB. The total study duration is up to approximately 400 days. A participant is considered to have completed the study if he/she has completed the Primary Evaluation Period.

Analysis of the Primary Evaluation Period is conducted when all participants have completed this period. In addition, 2 interim analyses of this study are conducted after approximately 30% and 50%, respectively, of the randomized participants have completed the Primary Evaluation Period, with the purpose of assessing early futility in the first interim analysis, and the need for sample size adjustment in the second interim analysis.

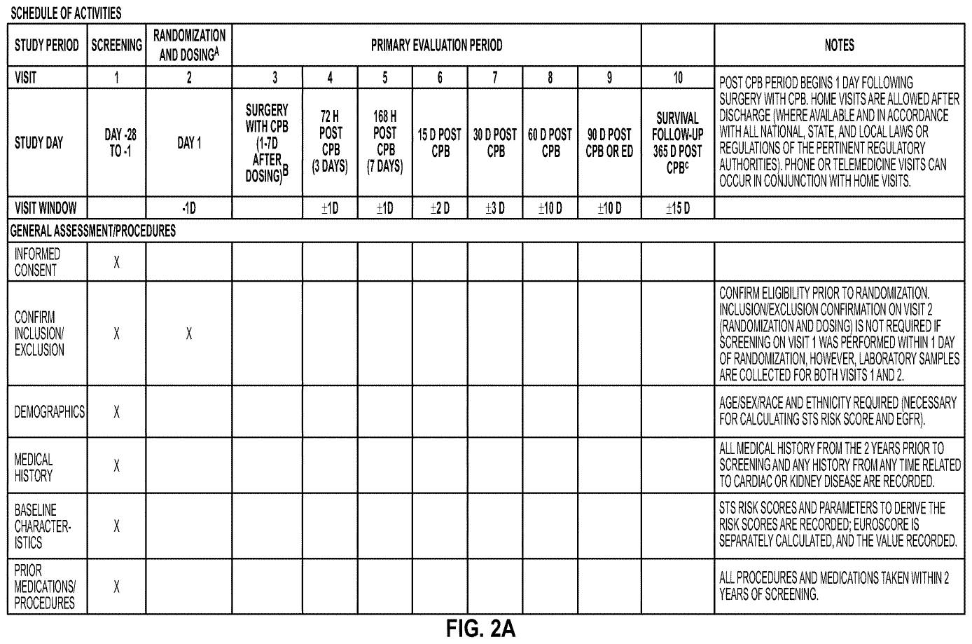

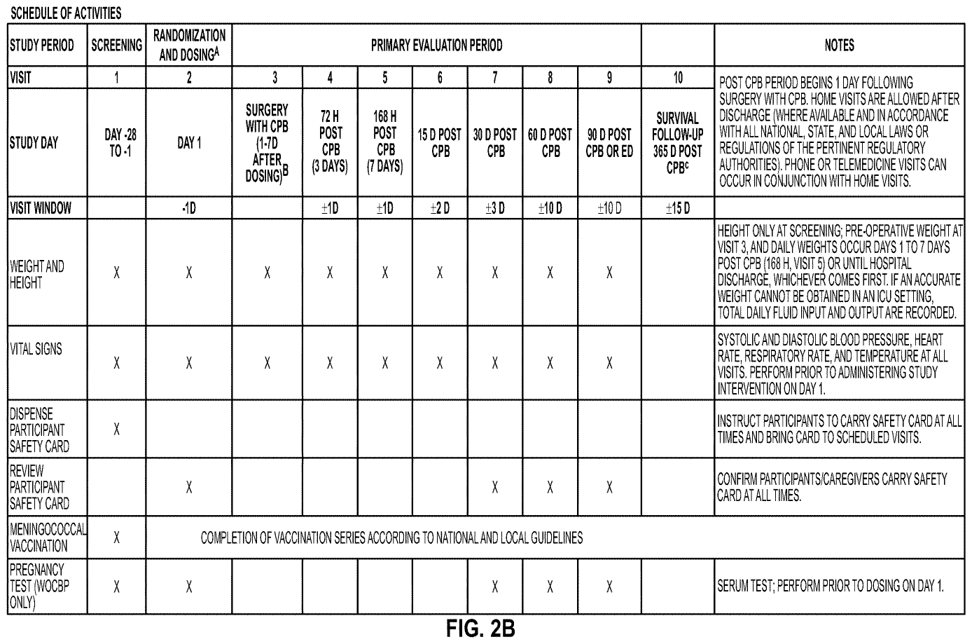

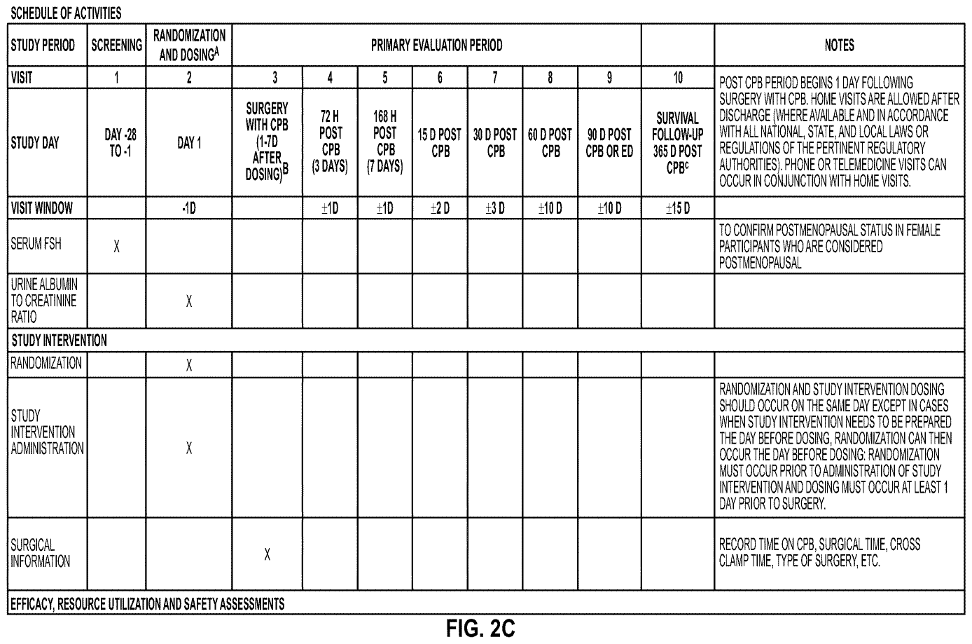

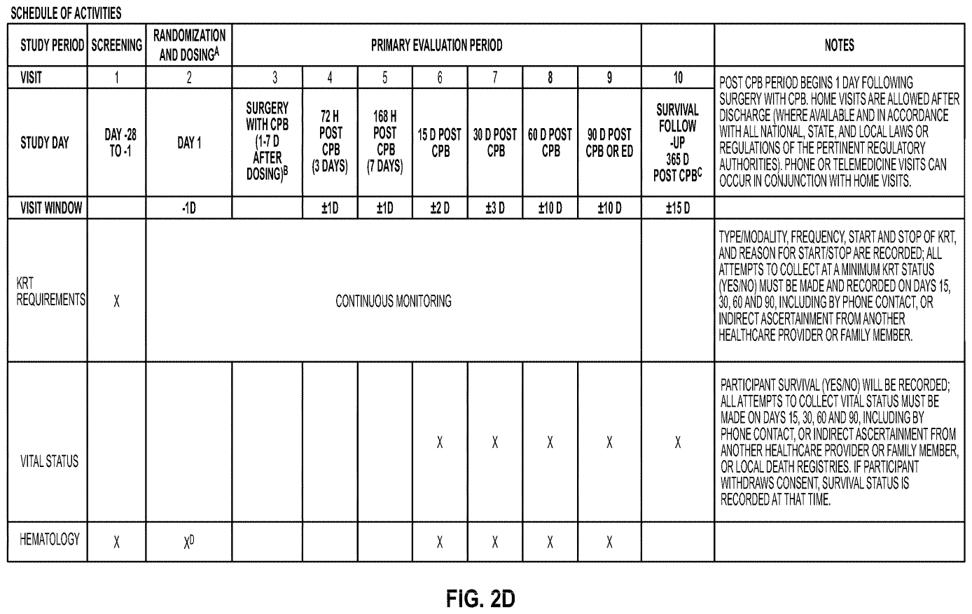

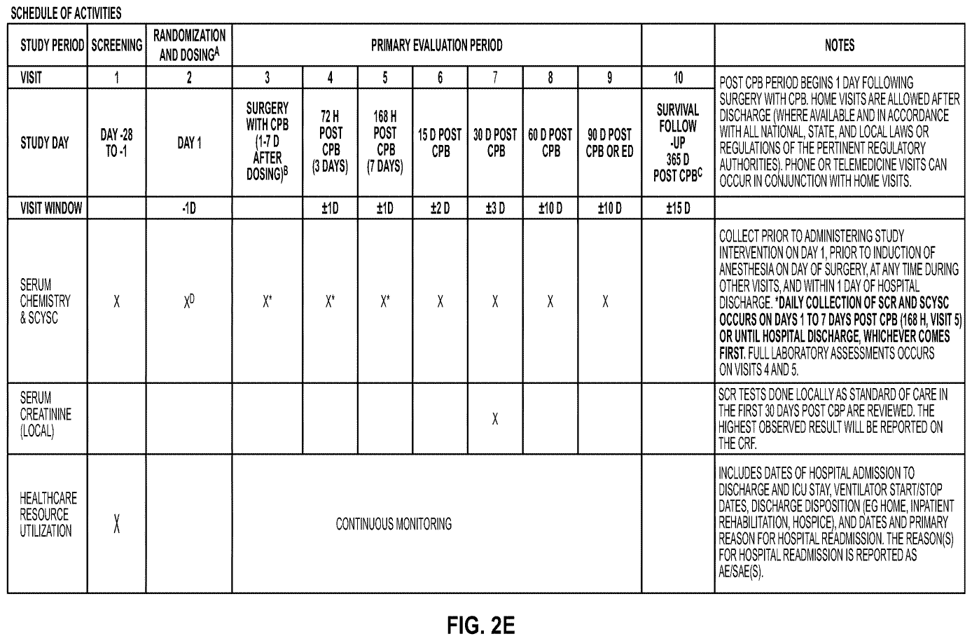

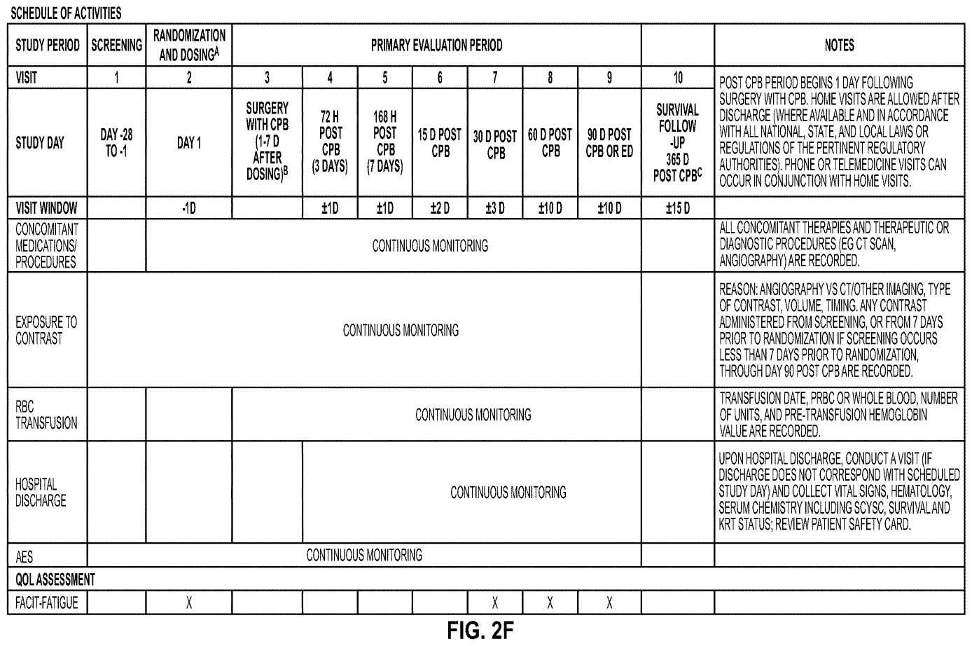

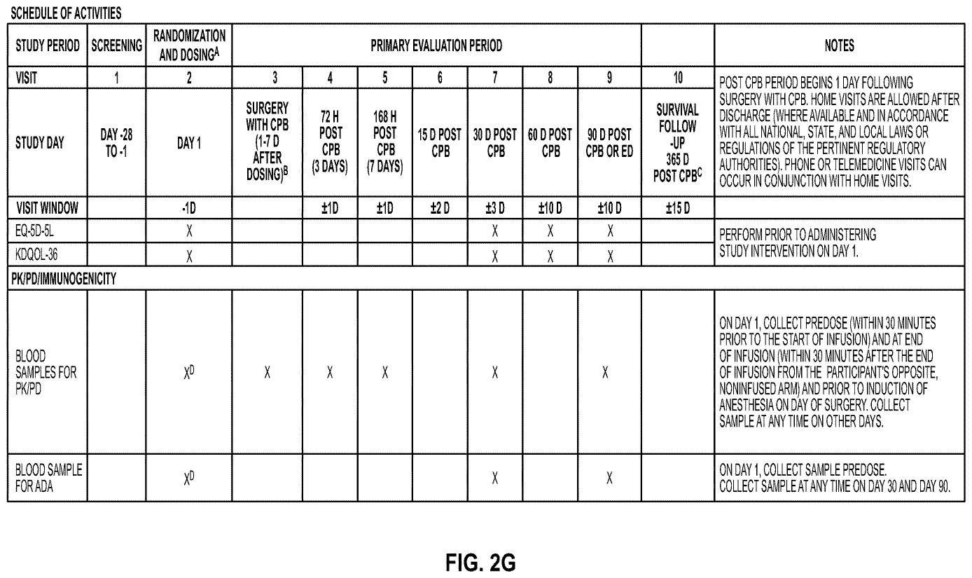

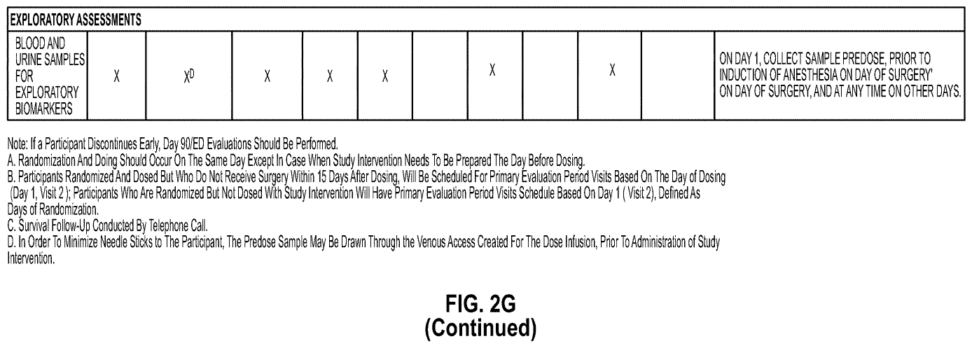

The end of the study is defined as the date the last participant completes the last visit as indicated in the Schedule of Activities (see FIGS. 2A-2G).

This is a parallel group treatment study with 2 arms that is participant and Investigator blinded, vRavulizumab is being evaluated for reducing risk of death, need for KRT, and sustained reduction in kidney function in adult patients with CKD undergoing CPB for CABG, valve replacement or repair, or combined procedure.

Randomized participants ill receive a single weight-based dose of ravulizumab or placebo within 1 to 7 days (i.e., at the latest 1 day prior to surgery) prior to CPB procedure.

### 2. Objectives, Estimands, and Endpoints

The primary objective of the study is to assess the efficacy of ravulizumab in reducing risk of MAKE90 following CPB. Primary estimands include:
(1) Treatment: ravulizumab or placebo;
(2) Population: Adult participants with CKD;
(3) Endpoint/Variables: MAKE at Day 90 post CPB (MAKE90), defined as meeting at least 1 of the following criteria: decrease from baseline in eGFR (CKD-EPI formula using sCysC) of ≥25% at Day 90 post CPB, or initiation of KRT through Day 90 post CPB, or death from any cause through Day 90 post CPB;
(4) Intercurrent events (IEs): IE1: Iodinated contrast dye exposure after treatment is administered through Day 90 post CPB; IE2: Surgery was performed without CPB or surgery with CPB was not performed within 15 days after treatment administration; and IE3: Use of confounding interventions prior to surgery or use of disallowed interventions after treatment is administered through Day 90 post CPB. Treatment policy strategy: the collected endpoint is analyzed irrespectively of IEs; and
(5) Summary measures: Difference between treatment groups in proportion of participants experiencing MAKE at 90 days post CPB, regardless of any IEs.

The key secondary efficacy objective of the study is to assess the efficacy of ravulizumab in reducing risk of AKI (based on sCr) following CPB based on: (1) CSA-AKI free at Day 90 post CPB, (2) free of severe CSA-AKI (KDIGO Stage 2 or 3) based on highest observed sCr within 7 days post CPB; (3) free of any severe AKI (RIFLE Injury or Failure criteria) based on highest observed sCr within Day 30 post CPB; (4) free of any severe AKI (KDIGO Stage 2 or 3) based on highest observed sCr within Day 30 post CPB; (5) free of any RIFLE Failure criteria based on highest observed sCr within Day 30 post CPB; and (6) All-cause mortality from randomization through Day 90 post CPB.

A further secondary efficacy objection is to assess the efficacy of ravulizumab in reducing risk of MAKE (based on sCysC), MAKE (based on sCr), AKI (based on sCr), and related outcomes following CPB. Corresponding endpoints and/or estimands include: (1) MAKE and its components at Days 30, 60 and 90 post CPB (excluding MAKE90 based on sCysC), Occurrence of KRT or death by Days 30, 60, 90 Post CPB. (2) highest CSA-AKI stage within 3 and 7 days post CPB, (3) CSA-AKI free at Day 15, 30 and 60 post CPB, (4) free of any AKI at Days 3, 7, 15, 30, 60, and 90 post CPB, and (5) AKI Progression on Days 15, 30, 60, and 90 post CPB for those experiencing CSA-AKI within 7 days post CPB: Complete recovery, Partial recovery, Improvement, Stable, or Worsening.

The Healthcare Resource Utilization objective is to assess the effect of ravulizumab on health resource utilization in participants with CKD undergoing non emergent CPB, as assessed by (1) length of index hospital and ICU stay, (2) number of ventilator-free days through Day 30 and Day 90 post CPB, (3) hospital readmission rate (all-cause or AKI-related) through Day 30 and Day 90 post CPB, and (4) days on KRT through Day 30 and Day 90 post CPB.

The Health-related QoL objective is to assess the effect of ravulizumab on quality of life in participants with CKD undergoing non emergent CPB, e.g., by (1) change from baseline in KDQOL-36™ at Days 30, 60, and 90 post CPB. (2) change from baseline in EQ-5D-5L at Days 30, 60, and 90 post CPB, and (3) change from baseline in FACIT-Fatigue at Days 30, 60, and 90 post CPB.

A further objective is to evaluate PK and PD of ravulizumab in participants with CKD undergoing non emergent CPB, e.g., via serum concentrations of ravulizumab and absolute values, change from baseline, and percent change from baseline in serum free C5 concentrations.

A further objective is to evaluate safety of ravulizumab IV in participants with CKD undergoing non emergent CPB, e.g., via Incidence of TEAEs and TESAEs and change from baseline in laboratory parameters at scheduled visits.

A further objective is to evaluate the immunogenicity of ravulizumab IV in participants with CKD undergoing non-emergent CPB, e.g., via ADA incidence. ADA response categories, and titer at Day 90 post CPB.

The exploratory objective is to assess biomarkers at baseline and change in response to treatment, e.g., via biomarker assessments, which can include, but are not limited to, complement pathway activation (e.g., plasma and urine soluble C5b-9), renal injury (e.g., urine neutrophil gelatinase-associated lipocalin [NGAL]) and endothelial damage (e.g., plasma thrombomodulin [TM]).

A final objective is to assess the efficacy of ravulizumab in reducing risk of CSA-AKI based on sCysC, e.g., via AKI within 7 days of CPB based on sCysC: Highest AKI stage by KDIGO criteria and free of severe AKI (KDIGO stage 2 or 3).

The Endpoint Definitions set forth in Table 1 are used in the study.

TABLE 1
Endpoint Definitions

| Endpoint(s) | Definition |
|---|---|
| MAKEXX (sCysC- | Meets at least 1 of the following criteria: |
| based), | Decrease from baseline in eGFR of ≥25% at Day XX |
| MAKEXX (sCr-based) | determined by the CKD-EPI formula using sCysC (for |
| (XX = 30, 60, or 90) | sCysC-based MAKEXX) or the CKD-EPI formula using sCr |
| | (for sCr-based MAKEXX), or |
| | Initiation of KRT through Day XX, or |
| | Death from any cause through Day XX |
| AKI (sCr-based), | Meets either of the following modified KDIGO criteria: |
| AKI (sCysC-based) | Increase in sCr (for sCr-based AKI) or sCysC (for sCysC-based |
| | AKI) by ≥0.3 mg/dL during any 48-hour interval |
| | Increase in sCr or sCysC to ≥1.5 × baseline within 3 and 7 days, |
| | or at Day 15, 30, 60 or 90 post CPB |
| AKI Stage (KDIGO | Meets modified KDIGO criteria for Stage 1, 2 or 3 as outlined in |
| criteria; sCr-based) | Table 2 |
| CSA-AKI free (sCr- | Meets either of the following criteria: |
| based) at Day XX post | Not experiencing CSA-AKI within 7 days post CPB |
| CPB | Achieving AKI recovery at Day XX after experiencing CSA- |
| (XX = 15, 30, 60, or 90) | AKI within 7 days post CPB |
| Severe CSA-AKI | Stage 2 or 3 AKI within 7 days after CPB based on highest observed |
| (KDIGO criteria; sCr- | sCr |
| based) | |
| Severe AKI (KDIGO | Meets modified KDIGO criteria for Stage 2 or 3 AKI as outlined in |
| criteria; sCr-based) | Table 2 based on highest observed sCr within 30 days post CPB |
| Severe AKI and Failure | Meets modified RIFLE criteria for Injury or Failure and Failure |
| (RIFLE criteria; sCr- | alone as outlined in Table 3 based on highest observed sCr within 30 |
| based) | days post CPB |
| Free of any AKI | sCr <1.5 × baseline at Day XX post CPB |
| (sCr-based) at Day XX | |
| post CPB | |
| (XX = 3, 7, 15, 30, 60, | |
| or 90) | |
| AKI recovery (sCr- | Complete: |
| based) | sCr <1.1 × baseline |
| | Partial: |
| | sCr ≥1.1-<1.5 × baseline |
| AKI improvement | Move down 1 or more stages from highest CSA-AKI (except |
| (sCr-based) | stage 0) as outline in Table 4 or |
| | Remain in Stage 3 but discontinue KRT |
| Stable AKI (sCr-based) | Unchanged AKI or KRT status |
| Worsening AKI (sCr- | Move up 1 or more stages from highest CSA-AKI stage, |
| based) | or |
| | Remain in Stage 3 but initiate KRT |

Abbreviations:
AKI = acute kidney injury;
CKD-EPI = Chronic Kidney Disease Epidemiology Collaboration;
CPB = cardiopulmonary bypass;
CSA-AKI = cardiac surgery-associated acute kidney injury;
eGFR = estimated glomerular filtration rate;
KDIGO = Kidney Disease - Improving Global Outcomes;
KRT = kidney replacement therapy;
MAKE = major adverse kidney event;
RIFLE = Risk, Injury, Failure, Loss of kidney function, and End-stage kidney disease;
sCr = serum creatinine;
sCysC = serum cystatin C

The primary estimand of this study uses the treatment policy strategy based on the Intent to Treat analysis set to estimate the treatment effect regardless of any intercurrent events (IEs) and participant compliance with the IP dosing. The estimand is intended to provide a population level estimate of the treatment effect on a binary endpoint MAKE90 post CPB, amongst CKD patients who meet study eligibility criteria and are randomized into the study. The primary estimand includes the following 4 attributes:
A. Population: Adult participants with CKD as defined by the inclusion and exclusion criteria
B. Variable: MAKE at Day 90 post CPB (MAKE90)
C. Intercurrent events (IEs):
   IE1: Iodinated contrast dye exposure after treatment is administered through Day 90 post CPB
   IE2: Surgery was performed without CPB or surgery with CPB was not performed within 15 days after treatment administration
   IE3: Use of confounding interventions prior to surgery or use of disallowed interventions after treatment is administered through Day 90 post CPB. Note: Treatment policy strategy: the collected endpoint are analyzed irrespectively of IEs.
D. Summary measures: Difference between treatment groups in proportion of participants experiencing MAKE at 90 days post CPB regardless of any IEs.

The key secondary estimands are intended to provide a population level estimate of the treatment effect on 5 binary endpoints, regardless of any IEs. The estimands for each key secondary will target the same estimand as for primary endpoint as follows:
A. Population: Adult participants with CKD as defined by the inclusion and exclusion criteria
B. Variable:
   CSA-AKI free at Day 90 post CPB;
   Free of severe CSA-AKI (Kidney Disease Improving Global Outcomes [KDIGO] Stage 2 or 3) based on highest observed sCr within 7 days post CPB Free of any severe AKI (Risk, Injury, Failure, Loss of kidney function, and End stage kidney disease [RIFLE] Injury or Failure criteria) based on highest observed sCr within Day 30 post CPB;
   Free of any severe AKI (KDIGO Stage 2 or 3) based on highest observed sCr within Day 30 post CPB;
   Free of any RIFLE Failure criteria based on highest observed sCr within Day 30 post CPB;
   All-cause mortality from randomization through Day 90 post CPB
C. Intercurrent events (IEs):
   IE1: Iodinated contrast dye exposure after treatment is administered through Day 90 post CPB;
   IE2: Surgery was performed without CPB or surgery with CPB was not performed within 15 days after treatment administration;
   IE3: Use of confounding interventions prior to surgery or use of disallowed interventions after treatment is administered through Day 90 post CP

Treatment policy strategy: the collected endpoint is analyzed irrespectively of IEs. Summary measures: Difference between treatment groups in proportion of participants experiencing key secondary endpoint event.

### 3. MAKE and CSA-AKI

The primary outcome measure (MAKE) was conceived to capture the clinical outcomes after AKI and is defined as mortality, need for KRT, and SKD defined as estimated glomerular filtration rate (eGFR)>25% below baseline by the CKD-EPI formula (see, e.g Billings F T, et al., Nephron. Clin. Pract. 2014; 127(1-4):89-93; Haverich A, et al., Ann. Thorac. Surg. 2006; 82(2):486-492; Levey A S, et al., Ann. Intern. Med. 2009:150(9):604-612; and Palevsky P M, et al., Clin. J. Am. Soc. Nephrol. 2012; 7(5):844-850). SKD occurs due to AKI non-recovery, and at 90 days represents a sustained worsening of CKD. SKD is associated with multifold higher risk for long-term mortality and progression to ESKD when compared to those who were AKI free, or who recovered from AKI (see, e.g., Ishani A, et al., J Am. Soc. Nephrol. 2009; 20(1):223-228; Wu V C, et al., Kidney Int. 2011; 80(11):1222-1230; and Cho J S, et al., J. Thorac. Cardiovasc. Surg. 2021; 161(2):681-8).

Skeletal muscle atrophy is widely recognized as a complication after cardiac surgery (van Venrooij L M, et al., Nutrition (Burbank, Los Angeles County, Calif). 2012; 28(1):40-45) and can confound sCr-based assessment of eGFR. As recommended for such circumstances in the KDIGO guidelines (KDIGO, 2013), sCysC is used for calculating SKD in the MAKE endpoint. Data from observational and randomized controlled interventional trials reporting AKI and MAKE outcomes after cardiopulmonary bypass were used to inform the MAKE 90 placebo rate assumption of 25%.

The occurrence of post-operative CSA-AKI is defined as the presence of one of the following observations during the 7 days after surgery with CPB, based on modified KDIGO criteria: (1) an increase in sCr by ≥0.3 mg/dL in a 48-hour period or (2) an increase in sCr to ≥1.5 times baseline within the 7 days following CPB.

The highest AKI stage by modified KDIGO criteria that occurs within 3 and 7 days after surgery with CPB is determined, as defined in Table 2 (see, also KDIGO., Kidney Inter. Suppl. 2013; 3:1-150 and Khwaja A., Nephron. Clin. Pract. 2012; 120(4):c179-184). Additionally, this staging is used to assess AKI at any time within 30 days after CPB based on the highest observed sCr; and at Days 15, 30, 60 and 90 after CPB. While the KDIGO criteria is widely adopted, in some settings (e.g., STS renal failure risk calculator) there is greater familiarity with the RIFLE criteria (e.g., O'Brien S M, et al., Ann Thorac Surg. 2018; 105(5):1419-1428). Therefore, AKI staging based on the modified RIFLE criteria is assessed for AKI at any time within 30 days after CPB based on the highest observed sCr, and the staging criteria is outlined in Table 3 (see, also Bellomo R, et al., Acute Dialysis Quality Initiative workgroup. Acute renal failure—definition, outcome measures, animal models, fluid therapy and information technology needs: the Second International Consensus Conference of the Acute Dialysis Quality Initiative (ADQI) Group. Crit Care. 2004; 8(4):R204-R212). In the setting of AKI, reliable interpretation of changes in sCysC are exploratory in this study as it is yet to be defined and is not commonly used to define or stage AKI at this time.

TABLE 2
Acute Kidney Injury (AKI) Staging by Modified KDIGO Criteria

| AKI Stage | Definition |
|---|---|
| 1 | An increase in serum creatinine (sCr) by ≥0.3 mg/dL |
| | (up to <2.0 × baseline) in a 48-hour period, or |
| | An increase in sCr to ≥1.5-<2.0 × baseline |
| 2 | An increase in sCr to ≥2.0-<3.0 × baseline |
| 3 | An increase in sCr to ≥3.0 × baseline, or |
| | An increase in sCr to ≥4.0 mg/dL in patients |
| | meeting AKI criteria, or Initiation of kidney |
| | replacement therapy (KRT). Note: Use of |
| | operative ultrafiltration as a standard of |
| | care procedure to routinely |
| | manage fluid balance during cardiac surgery |
| | is not considered initiation of KRT. |

TABLE 3
AKI Staging by Modified RIFLE Criteria

| AKI Stage | Definition |
|---|---|
| Risk | An increase in sCr to ≥1.5-<2.0 × baseline |
| Injury | An increase in sCr to ≥2.0-<3.0 × baseline |
| Failure | An increase in sCr to ≥3.0 × baseline, or |
| | An increase in sCr to ≥4.0 mg/dL with an |
| | increase of at least 0.5 mg/dL from baseline, or |
| | Initiation of KRT. Note: use of operative |
| | ultrafiltration as a standard |
| | of care procedure to routinely manage |
| | fluid balance during cardiac surgery |
| | is not considered initiation of KRT. |

Changes in the post-AKI progression (based on sCr or need for KRT) relative to the highest CSA-AKI stage observed in the first 7 days following CPB are assessed on Day 15 through Day 90 post CPB to characterize AKI progression (recovery, improvement, stable, or worsening). Stages of post-AKI progression are defined in Table 4 (see also Chawla L S. et al., Acute kidney disease and renal recovery: consensus report of the Acute Disease Quality Initiative (ADQI) 16 Workgroup. Nat Rev Nephrol. 2017; 13(4):241-257).

TABLE 4
Stages of Post-CSA-AKI Kidney Function to Determine AKI
Progression (e.g., on Day 15 to Day 90) After Surgery

| Post-AKI Stage | Definition |
|---|---|
| 0 | Complete recovery: sCr <1.1 × baseline |
| | Partial recovery: sCr ≥1.1-<1.5 × baseline |
| 1 | sCr ≥1.5-<2.0 × baseline |
| 2 | sCr ≥2.0-<3.0 × baseline |
| 3 | sCr ≥3.0 × baseline, or |
| | sCr ≥4.0 mg/dL in patients meeting |
| | CSA-AKI criteria, or KRT |

There are uncertainties in the underlying assumptions of the specific background MAKE rate in this at risk population, as well as in the treatment effect assumption. Therefore, two interim analyses of this study are conducted to determine futility and the need for sample size re-estimation.

The safety outcomes being evaluated are commonly used in clinical studies per International Council for Harmonisation of Technical Requirements for Pharmaceuticals for Human Use (ICH) and Good Clinical Practice (GCP) guidelines.

### 4. Justification for Dose

The dose in this study is identical to the weight-based ravulizumab maintenance dose approved for adults with aHUS, PNH or gMG and for which safety has been established.

This dose ensures that ravulizumab serum concentration is ≥175 μg/mL to achieve complete terminal complement inhibition, while maintaining maximum ravulizumab serum concentration below the highest observed value of 3000 μg/mL from all completed clinical studies, for at least 18 days in the target patient population. The 175 μg/mL threshold was previously identified as the therapeutic minimum concentration of ravulizumab required to achieve complete terminal complement inhibition.

To support this dose regimen, model-based simulations were conducted (Ravulizumab CSA-AKI Dose Rationale, 2022). Briefly, the population PK model used for the simulation was from aHUS label extension dossier. Factors that could potentially impact ravulizumab PK in the target patient population were integrated in the model simulation, and included impact of proteinuria, red blood cell transfusion and post-operative hemodilution.

Other nonquantifiable factors, such as drug loss or activated complement pathway by CPB, were not included in the simulation. As such, a dose that can achieve higher than 175 μg/mL ravulizumab concentration for at least 18 days is desirable in the setting of CSA-AKI and predicted with the proposed single ravulizumab weight-based doses.

A participant is considered to have completed the study if he/she has completed the Primary Evaluation Period. The end of the study is defined as the date the last participant completes the last visit as indicated in the Schedule of Activities.

### 5. Study Population

To be eligible to participate in the study, participants must meet all of the following criteria:

1. ≥18 to ≤90 years of age at the time of signing the informed consent.

2. Male or female. Female participants of childbearing potential and male participants must follow protocol specified contraception guidance.

3. Body weight ≥30 kg at Screening.

4. Planned non-emergent sternotomy with CPB procedure for the following surgeries: Multi-vessel CABG, valve replacement or repair; ascending aorta surgery permitted if combined with aortic valve replacement/repair, combined CABG and valve surgery; inclusion of single-vessel CABG when combined with valve replacement/repair is permitted. Note: surgery should be scheduled to occur within 35 days of screening (up to 28-day Screening Period with randomization and dosing within 1 to 7 days prior to CPB).

5. Known CKD (Stage 3A, 3B, or 4) for at least 3 months with eGFR≥20 to <60 mL/min/1.73 m2, eGFR confirmed at screening (may be repeated once during screening) and randomization using CKD-EPI equation by sCr or sCysC measurement, obtained by local or central laboratory.

6. At risk for post-surgical AKI as defined by a minimum STS Calculator Renal Failure Risk Score of 3%.

7. Capable of giving signed informed consent which includes compliance with the requirements and restrictions listed in the informed consent form and in this protocol.

Participants are excluded from the study if any of the following criteria apply:

1. Emergency or salvage cardiac surgery is expected at screening or randomization, as assessed by the Investigator.

2. Single-vessel CABG without valve surgery is planned.

3. Off-pump surgery is planned (e.g., surgery without CPB).

4. Any use of KRT or presence of AKI within 30 days of randomization (AKI defined as 1.5× increase in sCr over baseline), except transient (≤5 days) Stage 1 AKI after iodinated contrast exposure.

5. Recipient of a solid organ or bone marrow transplantation.

6. Cardiogenic shock, hemodynamic instability, use of intra-aortic balloon pump, extracorporeal membrane oxygenation, or left ventricular assist device within 72 hours of randomization.

7. Active systemic bacterial, viral, or fungal infection within 14 days prior to randomization.

8. Participants with history of human immunodeficiency virus (HIV) who are not on anti-retroviral therapy or if on therapy have a known detectable viral load within 1 year of Screening.

9. Congenital immunodeficiency.

10. History of unexplained, recurrent infection.

11. Known medical or psychological condition(s), including substance abuse, or risk factor that, in the opinion of the Investigator, might interfere with the participant's full participation in the study, pose any additional risk for the participant, or confound the assessment of the participant or outcome of the study.

12. History of or unresolved N. meningitidis infection.

13. Hypersensitivity to any ingredient contained in the study intervention, including hypersensitivity to murine proteins.

14. Current malignancy or receiving treatment for malignancy

15. Use of any complement inhibitors, or plasmapheresis or plasma exchange within the year prior to Screening. or planned use during the course of the study.

16. Planned use of any pharmacologic agent specifically for prevention or treatment of AKI.

17. Planned use of KRT, intra-aortic balloon pump, extracorporeal membrane oxygenation, or left ventricular assist device between randomization and surgery.

18. Participation in another interventional treatment study or use of any experimental therapy within 30 days before initiation of study intervention on Day 1 in this study or within 5 half-lives of that IP, whichever is greater, or planned participation/use during the course of the study.

19. Presence of a do-not-resuscitate order or life expectancy of <3 months.

20. Pregnant, breastfeeding, or intending to conceive within 8 months after the dose of study intervention.

21. Participant is not willing to be vaccinated against N meningitidis or is unwilling to receive prophylactic treatment with appropriate antibiotics, if needed.

Screen failures are defined as participants who consent to participate in the clinical study but are not subsequently randomly assigned to study intervention. A minimal set of screen failure information is required to ensure transparent reporting of screen failure participants to meet the Consolidated Standards of Reporting Trials (CONSORT) publishing requirements and to respond to queries from regulatory authorities. Minimal information includes demography, screen failure details (e.g., failed eligibility criteria), and any adverse events (AEs), including any serious adverse events (SAEs) and any related concomitant medication, occurring during the Screening Period.

Individuals who do not meet the criteria for participation in this study (screen failure) due to a reason that is expected to resolve or has resolved, may be rescreened based on discussion and agreement. Participants who are rescreened outside of the Screening window are required to sign a new informed consent form (ICF).

### 6. Study Intervention

Study intervention is defined as any investigational intervention(s), marketed product(s), or placebo intended to be administered to a study participant according to the study protocol.

Participants are randomized to either ravulizumab or placebo. Ravulizumab is formulated at pH 7.0 and is supplied in 30 mL single-use vials. Each vial of ravulizumab contains 300 mg of ravulizumab (10 mg/mL) in 10 mM sodium phosphate, 150 mM sodium chloride, 0.02% polysorbate 80, and water for injection. The comparator product (placebo) is formulated as a matching sterile solution with the same buffer components, but without active ingredient. Additional details are presented in Table 5.

TABLE 5
Study Interventions

| | | |
|---|---|---|
| Study Intervention | Ravulizumab | Placebo |
| Name | | |
| Dose formulation | Vial | Vial |
| Physical description | Liquid solution | Liquid solution |
| | practically free | practically free |
| | from particles | from particles |
| Unit dose Strength(s) | 300 mg (10 mg/mL | Placebo |
| | concentrated solution) | |
| Route of | Intravenous | Intravenous |
| administration | infusion | infusion |
| Use | Experimental | Placebo |
| | | comparator |

Participants randomized to the ravulizumab group receive a single weight-based dose of ravulizumab from 1 to 7 days (i.e., at least 1 calendar day) prior to CPB procedure. The dose is identical to the weight-based ravulizumab maintenance dose approved for adult patients with aHUS, PNH, or gMG (Table 6).

TABLE 6
Weight-based Single Dose of Ravulizumab or Placebo

| | |
|---|---|
| Body Weight | Dose |

| | | |
|---|---|---|
| ≥30 to <40 | kg | 2700 mg |
| ≥40 to <60 | kg | 3000 mg |
| ≥60 to <100 | kg | 3300 mg |
| ≥100 | kg | 3600 mg |

At a minimum, study intervention is labeled with: the protocol number, lot number/expiry date, name and address, and instructions for use and storage. The study intervention is labeled according to the country's regulatory requirements.

Upon arrival of the study intervention at the study site, the study intervention kits are removed from the shipping container and stored in their original cartons under refrigerated conditions at 2 °C to 8 °C (35 °F to 47 °F) and protected from light. Study intervention is not frozen. The Investigator must confirm appropriate temperature conditions have been maintained during transit for all study intervention received and that any discrepancies are reported and resolved before use of the study intervention. Study intervention is stored in a secure, limited-access storage area with temperature monitored daily.

Infusions of study intervention are prepared using aseptic technique. Ravulizumab and placebo is further diluted in a 1:1 ratio with compatible diluent. Ravulizumab and placebo is administered with a 0.2 micron filter during infusion.

Only participants enrolled in the study can receive the study intervention and only authorized site staff can supply or administer the study intervention. All study intervention must be stored in a secure, environmentally controlled, and monitored (manual or automated) area in accordance with the labeled storage conditions with access limited to the Investigator and authorized site staff.

In this study, participants in both treatment groups receive standard of care as background therapy.

Eligible participants are randomized either to the ravulizumab group or to the placebo group under a 1:1 allocation ratio. Randomization is performed centrally using Interactive Response Technology (IRT).

To balance the effects of potential confounding factors between the ravulizumab and placebo arms, randomization are stratified by baseline CKD stage (3A, 3B, 4) confirmed at screening and baseline based on CKD-EPI using sCr or sCysC (local or central laboratory result), and surgical type (mitral valve replacement or combined procedures vs other single procedure).

Participants, all investigative site personnel, and any designee, directly associated with the conduct of the study are blinded to participant treatment assignment throughout the study. The blinding is maintained by using identical study intervention kits and labels for ravulizumab and placebo. The placebo has an identical appearance to that of ravulizumab. The randomization code is maintained by the IRT provider.

The infusion of study intervention into participants is under the supervision of the Investigator or their designee to ensure that participants receive the appropriate dose at the appropriate time point during the study. The date and time of dose administration are recorded. Concomitant Therapy

Any medication or therapy (including over-the-counter or prescription medicines, vaccines, vitamins, and/or herbal supplements) deemed necessary for the participant's care during the study, or for the treatment of any adverse advent, along with any other medications, other than those listed as disallowed medications, can be given with discretion. It is the responsibility of the Investigator to record all medications along with: reason for use, dates of administration, including start and end dates, and dosage information including dose and frequency.

Participants are prohibited from receiving any of the following medications endpoints (Day 90 post CPB): eculizumab, ravulizumab (other than protocol-specified study intervention), or other agents that act on the complement pathway, plasmapheresis or plasma exchange, and use of any pharmacologic agent specifically for prevention or treatment of AKI (e.g., experimental or investigational, fenoldapam, levosimenden, and neseritide, etc). These therapies can be used for their approved indications.

The use of KRT, intra-aortic balloon pump, extracorporeal membrane oxygenation, or left ventricular assist should be avoided after study intervention is administered and before surgery unless clinically indicated. Use of these procedures after study intervention is administered and before surgery are considered confounding procedures, as these procedures can trigger AKI prior to the CPB or confound appropriate diagnosis and staging of AKI (e.g., use of KRT). These procedures are considered standard of care during the operative and post-operative phases and are permitted.

Dose modification of the study intervention for an individual participant is not permitted for this study.

### 7. Study Assessments and Procedures

Study procedures and their timing are summarized in the Schedule of Assessments set forth in FIG. 2. Protocol waivers or exemptions are not allowed. Adherence to the study design requirements, including those specified in the Schedule of Assessments is essential and required for study conduct. All screening evaluations must be completed and reviewed to confirm that potential participants meet all eligibility criteria. A screening log is maintained to record details of all participants screened and to confirm eligibility or record reasons for screening failure, as applicable.

Procedures conducted as part of the participant's routine clinical management (e.g., blood count) and obtained before signing of the informed consent form may be utilized for screening purposes provided the procedures met the protocol specified criteria and were performed within the time frame defined in the Schedule of Assessments.

Repeat or unscheduled samples can be obtained for safety reasons or for technical issues with the samples.

The Investigator or qualified designee must obtain a signed and dated informed consent form for each participant prior to conducting any study related procedures. All efforts should be made to ensure participants comply with study participation prior to conducting the screening procedures.

All inclusion and exclusion criteria must be reviewed by the Investigator or qualified designee to ensure the participant qualifies for study participation. The Investigator maintains a screening log to record details of all participants screened and to confirm eligibility or record reasons for screening failure, as applicable. Eligibility for participation is determined prior to randomization.

The study is intended to enroll non-emergent cardiac surgery participants, as judged by the Investigator. For guidance, elective cardiac surgery is described by Bojar R M., “Manual of Perioperative Care in Adult Cardiac Surgery”. Sixth Edition 2021 Print ISBN: 9781119582557, and as follows: T\the patient's cardiac function has been stable in the days or weeks prior to the operation and/or the procedure could be deferred without increased risk of compromised cardiac outcome.

Urgent cardiac surgery is defined as a procedure that is required during the same hospitalization in order to minimize chance of further clinical deterioration. This includes but is not limited to: worsening or sudden chest pain, heart failure, acute myocardial infarction, the underlying anatomy, unstable angina with intravenous nitroglycerin (IV NTG), rest angina. Any of these conditions that require that the patient remain in the hospital until surgery can take place, but the patient is able to wait for surgery until the next available OR schedule time. Delay in the operation may be necessitated by attempts to improve the patient's condition, availability of a spouse or parent for informed consent, availability of blood products, or the availability of results of essential laboratory procedures or tests. The definition for urgent procedures includes use of IABP, however, use or planned use of IABP is separately excluded in this study.

Participants requiring emergent or salvage cardiac surgeries, as judged by the Investigator, are not enrolled. For guidance, emergent cardiac surgery is defined as surgery that is indicated without any delay for ongoing, refractory (difficult, complicated, and/or unmanageable) unrelenting cardiac compromise, with or without hemodynamic instability, and not responsive to any form of therapy except cardiac surgery. Examples include hemodynamic picture of shock that is being chemically or mechanically supported, such as IV inotrope or IABP to maintain cardiac output, pulmonary edema requiring intubation and ventilation, an extending myocardial infarction, signs of ongoing ischemia, i.e., ECG changes, acute native valve dysfunction, (acute papillary muscle rupture or tom leaflet), prosthetic valve dysfunction with structural failure (valve-fractured or torn leaflet, thrombus formation, pannus development which impedes flow through the valve orifice, or valvular dehiscence), acute aortic dissection, rupture or dissection during cardiac catheterization; and perforation, tamponade following cardiac catheterization.

Salvage cardiac surgery is defined as a patient that is undergoing CPR en route to the OR prior to anesthesia induction or has ongoing ECMO to maintain life.

Demographic parameters, including age, sex, race, and ethnicity (necessary for calculating STS risk score and eGFR) are documented in the clinical study report (CRF).

Due to its mechanism of action, the use of ravulizumab increases a participant's susceptibility to meningococcal infection due to N meningitidis. To reduce the risk of infection, all participants must be vaccinated against N meningitidis within 3 years prior to study intervention administration. I f the participant is not vaccinated within 3 years of study intervention administration, vaccination occurs during screening or any time up to hospital discharge. If study intervention administration occurs <2 weeks after initial vaccination, participants should be administered prophylactic antibiotics for meningococcal infection up to 2 weeks after vaccination. Hospitalized participants may be vaccinated after study intervention administration but prior to hospital discharge. These participants receive prophylactic treatment with appropriate antibiotics from the day of dosing for at least 2 weeks after vaccination.

Vaccines against serotvpes A, C. Y, W135, and where available serotype B (if recommended by local guidelines), are recommended in preventing the commonly pathogenic meningococcal serotypes. Participants must receive the complete primary vaccination series and be revaccinated if indicated according to current national vaccination guidelines. Vaccination may not be sufficient to prevent meningococcal infection.

Use of prophylactic antibacterial agents should follow official guidance and local practice. All participants are monitored for early signs of meningococcal infection, evaluated immediately if infection is suspected, and treated with appropriate antibiotics, if necessary.

To increase risk awareness and promote quick disclosure of any potential signs or symptoms of meningococcal infection experienced by the participants during the course of the study, participants are provided a Participant Safety Card to carry with them at all times. Additional discussion and explanation of the potential risks, signs, and symptoms occur at specific time points as part of the review of the Participant Safety Card and throughout the study as described in the Schedule of Assessments.

The participant's relevant medical history, including prior and concomitant conditions/disorders, treatment history, and disease status of relevant diseases id evaluated at Screening and documented in the source documents and CRF. All medical history and prior medications and procedures from the 2 years prior to Screening, and any history from any time related to cardiac or kidney disease, including identified cause(s) of the participant's kidney disease, are recorded. Any changes to medical history occurring during the Screening Period and prior to dosing of study intervention on Day 1 are documented prior to study intervention administration.

A Society of Thoracic Surgeons (STS) risk calculator is utilized for determining pre-operative risk for severe AKI (RIFLE Failure criteria), based on the participant's baseline characteristics as required for the risk calculator. The risk score and the participant characteristics used to determine their risk score are captured during the Screening Period prior to dosing of study intervention on Day 1. EuroScore is separately calculated, and the value recorded.

Home visits are allowed after discharge where available. Home visits are conducted by qualified medical professionals in accordance with all national, state, and local laws or regulations of the pertinent regulatory authorities. Telemedicine visits may occur in conjunction with home visits.

All assessments may be performed at home visits under the direction of the Investigator. Information collected must be forwarded to the Investigator's site for evaluation on the day of the visit. In case of any symptoms or signs indicating a serious adverse event, further evaluation of the participant at the study site or an emergency care facility may be required.

### 8. Efficacy Assessments

For the primary efficacy endpoint, participants are evaluated for major acute kidney events (MAKE) at 90 days after cardiac surgery with CPB. MAKE90 is defined as meeting at least 1 of the following criteria: (1) decrease from baseline in eGFR (CKD-EPI formula using sCysC) of ≥25% at Day 90 post CPB, or (2) initiation of KRT through Day 90 post CPB, or (3) death from any cause through Day 90 post CPB.

Additionally, secondary endpoints assess both AKI and MAKE endpoints at additional timepoints. To enable these efficacy evaluations, the Investigator collects and records:
A. sCr and sCysC on day of screening, day of dosing (Day 1), day of surgery prior to induction of anesthesia, on Days 1-7, 15, 30, 60, and 90 post CPB, and upon hospital discharge: daily collection of sCr and sCysC occurs on Days 1 to 7 days post CPB (168 h, Visit 5) unless hospital discharge occurs before participant reaches Day 7 post CPB. However, presence of AKI wis followed with daily laboratory tests through Day 7 post CPB even if discharged, or until recovered (<1.5×baseline), whichever comes first.
B. The highest observed sCr by local laboratory from end of CPB through Day 30 is recorded.
C. Any use of KRT from randomization through Day 90 post CPB. Type/modality, frequency, start/stop of KRT, and reason for start/stop are recorded. All attempts to collect at a minimum KRT status (yes/no) must be made and recorded on Days 15, 30, 60 and 90. Use of operative ultrafiltration as a standard of care procedure to routinely manage fluid balance during cardiac surgery is not considered initiation of KRT, and is recorded as a concomitant procedure.
D. Any death from randomization through Day 90 post CPB. Date of death is collected and recorded, and any associated adverse events/serious adverse events including causal serious adverse events that result in the death are reported. All attempts to collect at a minimum vital status (survival, yes/no) must be made and recorded on Days 15, 30, 60 and 90

All efforts are made to collect laboratory test samples, KRT status, KRT details, and survival status at all visits. However, if needed. KRT status (yes/no). KRT details, and survival can be ascertained directly by phone or telemedicine visit. If attempts at direct contact with the participant fail, KRT status and survival status can be obtained indirectly from a family member, other healthcare provider, or local death registries.

Planned time points for all safety assessments are provided in the Schedule of Assessments.

Symptom-directed examinations can occur any time after screening as needed based on local practice/standard of care; normal findings or findings consistent with the participant's medical history is not recorded; abnormal findings not attributable to the participant's medical history, new abnormalities, or worsening of physical findings are reported as adverse events. Investigators must pay special attention to clinical signs related to previous serious illnesses. Height is only be measured at screening. Weight is measured prior induction of anesthesia on the day of surgery. Daily weight is measured in the first 7 days post CPB or until hospital discharge, whichever comes first. If an accurate weight cannot be obtained in an intensive care unit (ICU) setting, total daily fluid input and output is recorded in lieu of weight measurement. Temperature (°C or °F), heart rate, respiratory rate, and systolic and diastolic blood pressure (mmHg), are assessed. Blood pressure and pulse measurements are assessed with the participant in a seated position using a completely automated device. In the event the participant cannot tolerate measurements in the seated position (e.g., while on mechanical ventilation in the ICU), measurements can be performed and noted as recumbent. Manual techniques is used only if an automated device is not available. Blood pressure and pulse measurements are preceded by at least 5 minutes of rest for the participant in a quiet setting without distractions (e.g., television, cell phones). Ideally, the same arm for each participant is used for measurements. Vital signs are collected pre-dose on Day 1.

Symptom-directed electrocardiograms (ECGs) can occur any time after screening as needed based on local practice/standard of care. Normal findings or findings consistent with the participant's medical history are not recorded. Abnormal findings not attributable to the participant's medical history, new abnormalities, or worsening of previous ECG findings are reported as adverse events. Participants must be supine for approximately 5 to 10 minutes before ECG collection and remain supine but awake during ECG collection.

All protocol-required laboratory assessments are conducted in accordance with the Laboratory Manual and the Schedule of Assessments. Samples are collected for clinical laboratory assessments as follows: prior to administering study intervention on Day 1, prior to induction of anesthesia on day of surgery, at any time during other visits, and within 1 day of hospital discharge. Daily collection of samples for clinical laboratory assessments occurs on Days 1 to 7 days post CPB as follows: sCr and sCysC are collected on Days 1, 2, 4, 5, and 6 post CPB while hospitalized, along with full laboratory assessments on Days 3 and 7 post CPB, sCr and sCysC on Days 1, 2, 4, 5 or 6 do not need to be collected if hospital discharge occurs before Day 7 post CPB and the participant is free of AKI, a participant who develops any AKI within the first 7 days (at least Stage 1 by KDIGO criteria) is followed with daily laboratory tests regardless of discharge status through Day 7 post CPB or recovery (<1.5×baseline), whichever comes first. On Days 3 and 7, samples for clinical laboratory assessments are collected as described in the Schedule of Assessments.

All safety laboratory tests with values considered clinically significantly abnormal during participation in the study through the Day 90 Visit are repeated until the values return to normal or baseline or are no longer considered clinically significant. If such values do not return to normal/baseline within a period of time, the etiology should be identified.

If safety laboratory values from non-protocol specified laboratory assessments performed at the institution's local laboratory require a change in participant management or are considered clinically significant (e.g., adverse event or severe adverse event or dose modification), then the results are recorded in the CRF, and the corresponding adverse event or serious adverse event are reported.

Pregnancy testing is performed on all women of childbearing potential (WOCBP) at protocol-specified time points in the Schedule of Assessments. Pregnancy tests can also be performed at any time during the study. A negative pregnancy test is required for WOCBP before study intervention administration. Any female participant who becomes pregnant while participating in the study is discontinued from the study intervention. Pregnancy is not considered as an adverse event unless there is a suspicion that the study intervention may have interfered with the effectiveness of a contraceptive medication. However, complications of pregnancy and abnormal outcomes of pregnancy are adverse events and may meet the criteria for a serious adverse event (e.g., ectopic pregnancy, spontaneous abortion, intrauterine fetal demise, neonatal death, or congenital anomaly).

In the Primary Evaluation Period, vital status (survival yes/no) is documented at Days 15, 30, 60 and 90 after CPB. For participants who withdraw consent for further participation in the study, all efforts are made to collect data at withdrawal. In the Survival Follow-up Period, vital status is documented 365 days after CPB or ED, whichever comes first.

Vital status of participants may be obtained via telephone contact with the participant, participant's family, by contact with another healthcare provider, or local death registries.

### 9. Adverse Events (AE) and Serious Adverse Events (SAE)

An AE is any untoward medical occurrence in a clinical study participant administered a pharmaceutical product and which does not necessarily have to have a causal relationship with this treatment. An AE can therefore be any unfavorable and unintended sign (including an abnormal laboratory finding), symptom, or disease (new or exacerbated) temporally associated with the use of the study intervention, whether or not considered related to the study intervention.

The following events meet the AE definition:
A. Any abnormal laboratory test results (hematology, clinical chemistry, or urinalysis) or other safety assessments (e.g., ECG, radiological scans, vital signs measurements), including those that worsen from baseline, considered clinically significant (i.e., not related to progression of underlying disease).
B. Exacerbation of a chronic or intermittent pre-existing condition including either an increase in frequency and/or intensity of the condition.
C. New conditions detected or diagnosed after study intervention administration even though it may have been present before the start of the study.
D. Signs, symptoms, or the clinical sequelae of a suspected drug-drug interaction.
E. Signs, symptoms, or the clinical sequelae of a suspected overdose of either study intervention or a concomitant medication. Overdose per se is not reported as an AE/SAE unless it is an intentional overdose taken with possible suicidal/self-harming intent. Such overdoses are reported regardless of sequelae.

Events that do not meet the AE definition include:
A. Medical or surgical procedure (e.g., endoscopy, appendectomy): The condition that leads to the procedure is the AE, while the procedure itself is recorded as concomitant procedure. Situations in which an untoward medical occurrence did not occur (eg, hospitalization for elective surgery if planned before the signing the ICF, admissions for social reasons or for convenience).
B. Anticipated day-to-day fluctuations of pre-existing disease(s) or condition(s) present or detected at the start of the study that do not worsen.
C. A medication error (including intentional misuse, abuse, and overdose of the product) or use other than what is defined in the protocol is not considered an AE unless there is an untoward medical occurrence as a result of a medication error.
D. Cases of pregnancy that occur during maternal or paternal exposure to study intervention are reported within 24 hours of Investigator/site awareness. Data on fetal outcome and breastfeeding is collected for regulatory reporting and safety evaluation.
E. Any clinically significant abnormal laboratory findings or other abnormal safety assessments which are associated with the underlying disease, unless judged by the Investigator to be more severe than expected for the participant's condition.
F. The disease/disorder being studied or expected progression, signs, or symptoms of the disease/disorder being studied, unless more severe than expected for the participant's condition.
G. Situations in which an untoward medical occurrence did not occur (social and/or convenience admission to a hospital).
H. “Lack of efficacy” or “failure of expected pharmacological action” per se are not reported an AE or SAE. Such instances are captured in the efficacy assessments. However, the signs, symptoms, and/or clinical sequelae resulting from lack of efficacy are reported as AE or SAE if they fulfil the definition of an AE or SAE.

If an event is not an AE per definition above, then it cannot be an SAE even if serious conditions are met (e.g., hospitalization for signs/symptoms of the disease under study, death due to progression of disease).

An SAF is defined as any untoward medical occurrence that, at any dose, meets one or more of the criteria listed set forth in Table 7.

TABLE 7
SAE

1. Results in death
2. Is life-threatening
The term “life-threatening” in the definition of “serious” refers to an event in which the
participant was at risk of death at the time of the event. It does not refer to an event, which
hypothetically might have caused death, if it was more severe.
3. Requires inpatient hospitalization or prolongation of existing hospitalization
In general, hospitalization signifies that the participant has been detained (usually
involving at least an overnight stay) at the hospital or emergency ward for observation
and/or treatment that would not have been appropriate in the physician's office or
outpatient setting. Complications that occur during hospitalization are AEs. If a
complication prolongs hospitalization or fulfills any other serious criteria, the event is
serious. When in doubt as to whether hospitalization occurred or was necessary, the AE
should be considered serious.
Hospitalization for elective treatment of a pre-existing condition that did not worsen from
baseline is not considered an AE.
4. Results in persistent disability/incapacity
The term disability means a substantial disruption of a person's ability to conduct
normal life functions.
This definition is not intended to include experiences of relatively minor medical
significance such as uncomplicated headache, nausea, vomiting, diarrhea, influenza,
and accidental trauma (e.g., sprained ankle) which may interfere with or prevent
everyday life functions but do not constitute a substantial disruption.
5. Is a congenital anomaly/birth defect
6. Other situations:
Medical or scientific judgment should be exercised in deciding whether SAE reporting
is appropriate in other situations such as important medical events that may not be
immediately life-threatening or result in death or hospitalization but may jeopardize
the participant or may require medical or surgical intervention to prevent one of the
other outcomes listed in the above definition. These events should usually be
considered serious.
Examples of such events include invasive or malignant cancers, intensive treatment in
an emergency room or at home for allergic bronchospasm, blood dyscrasias or
convulsions that do not result in hospitalization, or development of drug dependency
or drug abuse.

A suspected unexpected serious adverse reaction (SUSAR) is defined as an event that is assessed as serious that is not listed in the appropriate Reference Safety Information (IB) and has been assessed that there is at least a reasonable possibility that the event is related to the investigational medicinal product.

The Investigator makes an assessment of intensity for each AE and SAE reported during the study and assign it to one of the following categories from National Cancer Institute CTCAE v5.0, published 27 Nov. 2017: Grade 1: Mild (awareness of sign or symptom, but easily tolerated), Grade 2: Moderate (discomfort sufficient to cause interference with normal activities), Grade 3: Severe (incapacitating, with inability to perform normal activities), Grade 4: Life-threatening, or Grade 5: Fatal. An event is defined as “serious” when it meets at least one of the predefined outcomes as described in the definition of an SAE, not when it is rated as severe.

The investigator is obligated to assess the relationship between the study intervention and each occurrence of each AE or SAE. An Investigator causality assessment must be provided for all AEs (both nonserious and serious). This assessment must be recorded in the eCRF and on any additional forms, as appropriate. The definitions for the causality assessments are as follows:
A. Not related: There is no reasonable possibility the study intervention caused the AE. The AE has a more likely alternative etiology: it may be due to underlying or concurrent illness, complications, concurrent treatments, or effects of another concurrent drug. The event does not follow a reasonable temporal relationship to administration of the study intervention.
B. Related: There is a reasonable possibility the study intervention caused the AE. The AE has a temporal relationship to the administration of the study intervention. The event does not have a likely alternative etiology. The event corresponds with the known pharmaceutical profile of the study intervention. There is improvement on discontinuation and/or reappearance on rechallenge

The Investigator uses clinical judgment to determine the relationship. Alternative causes, such as underlying disease(s), concomitant therapy, and other risk factors, as well as the temporal relationship of the event to study intervention administration are considered and investigated. The Investigator also consults the IB and/or Product Information, for marketed products, in his/her assessment. For each AE/SAE, the Investigator must document in the medical notes that they have reviewed the AE/SAE and has provided an assessment of causality.

Intravenous and infusion-related reactions are a potential risk with the use of monoclonal antibodies; these reactions can be nonimmune or immune mediated (e.g., hypersensitivity reactions). Signs and symptoms may include headache, fever, facial flushing, pruritus, myalgia, nausea, chest tightness, dyspnea, vomiting, erythema, abdominal discomfort, diaphoresis, shivers, hypertension, lightheadedness, hypotension, palpitations, and somnolence. Signs and symptoms of hypersensitivity or allergic reactions may include hives, swollen face, eyelids, lips, or tongue, or trouble with breathing.

All administration, IV, and infusion-related reactions are reported to the Investigator and qualified designee. The Investigator and qualified designee are responsible for detecting, documenting, and recording events that meet the definition of AE or SAE and remain responsible for following up events that are serious, considered related to the study intervention, or study procedures; or that caused the participant to discontinue ravulizumab. Participants who experience a reaction during the administration of ravulizumab are treated according to institutional guidelines. Participants who experience a severe reaction during administration of ravulizumab resulting in discontinuation of ravulizumab undergo all scheduled safety, PK, and PD evaluations required by the protocol. All AEs that may indicate an infusion-related response are graded according to the Common Terminology Criteria for Adverse Events (CTCAE) v5.0 or higher.

If anaphylaxis occurs according to the criteria listed in Table 8, then administration of subcutaneous epinephrine (1/1000, 0.3 mL to 0.5 mL, or equivalent) is considered. In the case of bronchospasm, treatment with an inhaled beta agonist is also considered. Participants administered an antihistamine for the treatment or prevention of an infusion related reaction are appropriate warnings about drowsiness and impairment of driving ability before being discharged from the center.

TABLE 8
Clinical Criteria for Diagnosing Anaphylaxis
Anaphylaxis is highly likely when any 1 of the following 3 criteria is fulfilled:

Acute onset of an illness (minutes to several hours) with involvement of the skin,
mucosal tissue, or both (e.g., generalized hives, pruritus or flushing, swollen lips-
tongue-uvula), and at least 1 of the following:
Respiratory compromise (e.g., dyspnea, wheeze-bronchospasm, stridor,
reduced peak expiratory flow, hypoxemia)
Reduced blood pressure or associated symptoms of end-organ dysfunction
(e.g., hypotonia [collapse], syncope, incontinence)
Two or more of the following that occur rapidly after exposure to a likely allergen for
that participant (minutes to several hours):
Involvement of the skin-mucosal tissue (e.g., generalized hives, itch-flush,
swollen lips/tongue/uvula)
Respiratory compromise (e.g., dyspnea, wheeze/bronchospasm, stridor,
reduced peak expiratory flow, hypoxemia)
Reduced blood pressure or associated symptoms (e.g., hypotonia [collapse],
syncope, incontinence)
Persistent gastrointestinal symptoms (e.g., crampy abdominal pain, vomiting)
Reduced blood pressure after exposure to known allergen for that participant (minutes
to several hours):
Systolic blood pressure of less than 90 mmHg or greater than 30% decrease
from that participant's baseline

Meningococcal infections are considered to be adverse events of special interest (AESIs).

For this study, any dose of study intervention greater than that specified in the protocol is considered an overdose. If dose cannot be established due to blinding, suspected overdose is defined by volume administered.

Accidental overdose or suspected overdose without any association with laboratory abnormalities or clinical symptoms is not considered as an AE. Overdose must be reported by the Investigator within 24 hours regardless of its association with or without an AE.

Overdoses are medication errors that are not considered AEs unless there is an untoward medical occurrence resulting from the overdose.

### 10. Pharmacokinetics (PK) and Pharmacodynamics (PD)

Blood samples for determination of serum ravulizumab concentrations and PD assessments (free C5) are collected before and after administration of study intervention at the time points specified in the Schedule of Assessments. The actual date and time (24-hour clock time) of each sample is recorded.

The Day 1 baseline PK and PD blood samples are collected at pre-dose, within 30 minutes before administering study intervention at visits specified in the Schedule of Assessments. The Day 1 pre-dose blood sample can be drawn through the venous access created for the dose infusion, prior to administration of the dose.

Post-dose PK and PD blood samples are collected within 30 minutes after completing study intervention infusion. The post-dose blood samples are drawn from the participant's opposite, non-infused arm. Samples can be collected at any time on visits after Day 1. In the event of an unscheduled visit, PK and PD blood sample is collected as soon as possible.

Study intervention concentration information that may unblind the study are not reported to investigative sites or blinded personnel until the study has been unblinded.

Genetics are not evaluated in this study.

Blood (serum & plasma) samples for exploratory assessments are collected from all participants at the time points specified in the Schedule of Assessments. Biomarkers can include, but are not limited to, assessments of the following: (1) complement pathway activation (e.g., soluble C5b-9 [sC5b-9]) (2) endothelial damage and/or activation (e.g., thrombomodulin [TM])), (3) vascular inflammation (e.g., shed tumor necrosis factor receptor I [TNF-RI]), and (4) inducers of cell-cycle arrest (e.g., tissue inhibitor of metalloproteinase-2 [TIMP-2]).

Urine samples for exploratory assessments are collected from all participants at the time points specified in the Schedule of Activities. Biomarkers include, but are not limited to, assessments of the following: (1) complement pathway activation (e.g., sC5b-9), and (2) renal injury (e.g., neutrophil gelatinase-associated lipocalin [NGAL]).

Residual blood and urine samples from exploratory biomarkers. PK, PD, and immunogenicity, are stored for additional assessments (e.g., related to the study intervention target, disease process, pathways associated with disease state, other complement-related diseases, and/or mechanism of action of ravulizumab). Samples are retained no longer than 5 years after termination of the study or other period as per local requirements.

Quality of life scales are completed by the participant prior to other study procedures at visits specified in the Schedule of Assessments.

Participants in both cohorts have the following validated quality of life scales administered: Kidney Disease Quality of Life instrument-36 items (KDQOL-36) (Section 10.7), a 36 item short form survey, is a widely used measure for patients on dialysis. Participants are asked to answer questions about their health, kidney disease, and effects of kidney disease on daily life.

The European Quality of Life Group's 5 dimension 5-level (EQ-5D-5L) (Section 10.7) is a self-assessed, standardized instrument to measure health related quality of life and has been used in a wide range of health conditions. The EQ 5D 5L is a 5 scale participant reported outcome tool measuring pain/discomfort, mobility, self-care, usual activities and anxiety/depression.

The Functional Assessment of Chronic Illness Therapy (FACIT) Fatigue scale, version 4.0, is a 13-item questionnaire that assesses self-reported fatigue and its impact upon daily activities and function over the preceding 7 days.

Healthcare resource utilization associated with medical encounters is collected for all participants throughout the Primary Evaluation Period. Protocol-mandated procedures, tests, and encounters are excluded. Hospitalization from admission to discharge including admission date, discharge date as well as dates of ICU admission and discharge to another inpatient ward). Kidney replacement therapy (KRT): type/modality, frequency, start and stop dates, and reason for start/stop. Duration of mechanical ventilation (dates and start/stop time). Discharge destination (e.g., home, rehabilitation facility, hospice).

Hospital readmission (emergency room or in-patient hospitalization; elective outpatient procedures that occur at a hospital are not considered readmission). Primary reason is assessed as: pre-operation, which is always a primary reason, if present, cardiovascular event not requiring re-operation (for example, MI, stroke, heart failure, arrythmia), AKI, or other reason. Hospitalization from readmission to discharge including date presenting to Emergency Room, inpatient admission date, discharge date and dates of ICU admission and discharge to another inpatient ward. Specific reason for readmission is reported as AE/SAE.

Additional data collected (e.g., concomitant medication, as well as outpatient diagnostic and therapeutic procedures) can be used to conduct exploratory economic analyses.

Other exploratory endpoints include: (1) AKI within Day 7 post CPB based on sCysC. (2) highest AKI stage by KDIGO criteria, and (3) free of severe AKI (KDIGO stage 2 or 3).

The minimum follow-up for safety is 90 days from dosing of study intervention (Day 1, Visit 2). Participants in this study receive a single weight-based dose of study intervention and have cardiac surgery with CPB within 1 to 7 days of dosing (maximum interval between dosing and cardiac surgery with CPB is 15 days, should there be unexpected delay(s) to surgery) and have all study visits and procedures as outlined in Schedule of Assessments through the Day 90 visit (Visit 9). The timing of post-operative (post CPB) visits on Days 3, 7, 15, 30, 60 and 90 is based on day of CPB, and with Day 1 post CPB being defined as the day after the participant comes off CPB. However, additional scenarios might arise during the conduct of the study: If the participant is dosed study intervention and does not have cardiac surgery, or the surgery is delayed more than 15 days after dosing, the participant has all subsequent study visits and procedures as outlined in Schedule of Assessments through the Day 90 visit, with the timing of visits based on day of dosing (Day 1, Visit 2). If the participant is randomized but not dosed, the participant has all study visits and procedures as outlined in Schedule of Assessments through the Day 90 visit, with timing of visits based on day of randomization (which is Day 1, Visit 2). If the participant withdraws, the decision for early discontinuation (ED) is made, Visit 9 is immediately conducted.

Antidrug antibodies to ravulizumab (i.e., antidrug antibody) is evaluated in serum samples collected from all participants according to the Schedule of Assessments. Additionally, serum samples are collected at the final visit from participants who discontinued study intervention or were withdrawn from the study.

Serum samples are screened for antibodies binding to ravulizumab and the titer of confirmed positive samples is reported. Other analyses can be performed to further characterize the immunogenicity of ravulizumab.

The detection and characterization of antibodies to ravulizumab is performed using a validated assay method. Samples collected for detection of antibodies to ravulizumab are evaluated for ravulizumab serum concentration to assess the impact of ADA on drug concentration profiles of ADA positive patients. ADA positive samples are further characterized for antibody titer and presence of neutralizing antibodies.

ADA variables include ADA response category incidence and titer over the duration of the study as follows. ADA response category definitions and titer thresholds are provided in the statistical analysis plan (SAP). ADA response categories are ADA negative and ADA positive. Participants who are ADA positive are categorized as follows: pre-existing immunoreactivity or treatment-emergent ADA responses.

### 11. Statistical Considerations

The primary hypothesis to be tested for this study is that ravulizumab is superior to placebo in reducing the risk of MAKE event at 90 days following CPB (MAKE90):

p₁≥p₀ H₀:

p₁<p₀ H₁:
where p₁ and p₀ represent the probability of experiencing MAKE90 event, in ravulizumab group and placebo group, respectively.

The treatment effect based on the MAKE90 endpoint is estimated by the difference in the proportion of participants experiencing MAKE90 regardless of any intercurrent events (IEs) between the ravulizumab group and the placebo group. A negative difference indicates a beneficial treatment effect for ravulizumab.

The following key secondary hypotheses are tested sequentially in the following hierarchical order, given the null hypothesis for the primary endpoint is rejected. Ravulizumab is superior to placebo in the proportion of participants who are CSA AKI free at Day 90 post CPB. Ravulizumab is superior to placebo in the proportion of participants who are free of severe CSA-AKI (KDIGO Stage 2 or 3) based on highest observed sCr within 7 days post CPB. Ravulizumab is superior to placebo in the proportion of participants who are free of severe AKI (RIFLE Injury or Failure criteria) based on highest observed sCr within 30 days post CPB. Ravulizumab is superior to placebo in the proportion of participants who are free of any severe AKI (KDIGO Stage 2 or 3) based on highest observed sCr within 30 days post CPB. Ravulizumab is superior to placebo in the proportion of participants who are free of any RIFLE Failure criteria based on highest observed sCr within 30 days post CPB. Ravulizumab is superior to placebo in the proportion of participants who died from any cause from randomization through Day 90 post CPB.

The sample size estimation was based on two-proportion difference using normal approximation method to compare the treatment difference in the proportion of participants experiencing MAKE events within 90 days post CPB between the ravulizumab and the placebo group. A sample size of 736 (368 participants per treatment group) has 90% power to detect a statistically significant treatment difference of 10% in the proportion of participants with MAKE within 90 days post CPB under a 2-sided significance level of 0.05, assuming the proportion of MAKE is 25% for the placebo group and 15% for the ravulizumab group with an approximate 10% dropout rate. The assumption of 25% MAKE90 rate in placebo group was derived based on recent interventional and observational trials as described below.

Recent interventional trials aimed at reducing CSA-AKI reported 9% to 10% of participants receiving placebo achieving MAKE endpoint at Day 30 (see. e.g., Meersch M, et al., Intensive Care Med. 2017; 43(11):1551-61), Jacob K A, et al., J. Am. Soc. Nephrol. 2015; 26(12):2947-51; Venugopal H, et al., Kidney360. 2020; 1(6):530-3) and 13% to 22% (see, e.g., Thielmann M, et al., Circulation. 2021; 144(14):1133-1144; and Meersch M, et al., Intensive Care Med. 2017; 43(11):1551-61)) at Day 90. These studies included a variable proportion of participants with CKD (8% to 47%). A post-hoc analysis of the subgroup with CKD (pre-surgical eGFR<60 mL/min/1.73 m2) from the Dexamethasone for Cardiac Surgery study reported that MAKE30 was 16.5% in the placebo group (see Venugopal H, et al., Kidney360. 2020; 1(6):530-3). Since these studies did not exclusively enroll CKD patients, a higher placebo rate is expected for this study with enrollment of individuals at increased risk for AKI with pre-existing CKD and heightened predicted risk of renal failure using the STS risk calculator.

There is limited data from the other trials on the frequency of MAKE (or its individual component outcomes) for those with pre-existing CKD. Therefore, estimates of individual MAKE events were derived from observational studies along with this trial data to inform the placebo rate assumption of 25% for the current study:

KRT 7% to 10%: Interventional trials reported KRT in 6.5% to 7.5% of participants receiving placebo (see. e.g., Thielmann M, et al., Circulation. 2021; 144(14):1133-1144; and Meersch M, et al., Intensive Care Med. 2017; 43(11):1551-61)). Observational studies from a variety of healthcare systems report post-operative KRT in 7% to 30% of patients with pre-existing CKD (see, e.g., Wu V C, Kidney Int. 2011; 80(11):1222-1230; Cho J S, et al., J. Thorac. Cardiovasc. Surg. 2021; 161(2):681-8.e3; and Lau D, et al., J Thorac. Cardiovasc. Surg. 2021; 162(3):880-7). By exclusively recruiting participants with CKD, at least 7% of participants requiring KRT is expected. Use of the STS renal failure risk calculation threshold for inclusion in the study is also expected to enrich for participants with highest risk of requiring KRT, especially amongst participants with pre-existing CKD stage 3a.

Mortality 4% to 7%: Interventional trials report mortality rates of 2% to 7% within 90 days following CPB (see, e.g., Thielmann M, et al., Circulation. 2021; 144(14):1133-1144; and Meersch M, et al., Intensive Care Med. 2017; 43(11):1551-61); Whitlock R P, et al. Lancet. 2015; 386(10000):1243-53; and Dieleman J M, et al., JAMA. 2012; 308(17):1761-7). In observational studies, AKI is independently associated with risk of mortality after CPB, with the highest risk in patients requiring acute KRT (see, e.g., Lau D, et al., J. Thorac. Cardiovasc Surg. 2021:162(3):880-7; and Matsuura R, et al., Sci Rep. 2020:10(1):6490). Additionally, AKI super-imposed on pre-existing CKD confers a higher risk of mortality following cardiac surgery (up to 11%) (Cho 2021). Therefore, by enrolling exclusively CKD patients overall mortality of approximately 5% to 6% is expected.

SKD 17% to 25%: Using changes in sCr or sCr-based eGFR, 19% to 22% of the CKD populations reported in observational studies have SKD by 3 months following CPB (see, e.g., Wu V C, Kidney Int. 2011; 80(11):1222-1230; Xu J, et al., BMC Nephrol. 2019; 20(1):427; Matsuura R, et al., Sci Rep. 2020; 10(1):6490); and Cho J S, et al., J. Thorac. Cardiovasc. Surg. 2021; 161(2):681-8.e3). AKI superimposed on CKD is the largest risk factor for SKD with 20% to 30% experiencing non-recovery back to baseline kidney function by 90 days (Cho 2021, Wu, 2011, Matsuura 2020). There are limited data from the interventional trials to directly inform the rate of SKD in participants with pre-existing CKD. Recent trials have reported SKD rates of 7% to 12% in control/placebo groups without fully enriching for CKD see, e.g., Thielmann M, et al., Circulation. 2021; 144(14):1133-1144; and Meersch M, et al., Intensive Care Med. 2017; 43(11):1551-61). The exclusive enrollment of participants with CKD is expected to increase the proportion of participants classified with SKD at 90 days compared to other CSA-AKI trials.

MAKE90 20% to 30%: The frequency of individual events is not additive since an individual participant can often meet criteria for >1 event within 90 days. Recent clinical trials (see, e.g., Thielmann M, et al., Circulation. 2021; 144(14):1133-1144; and Meersch M, et al., Intensive Care Med. 2017; 43(11):1551-61) reported approximately 30% of patients experienced more than 1 component of the MAKE (KRT. Death, SKD). Assuming a similar pattern in the current study, the expected proportion of participants in the placebo group achieving the MAKE endpoint at 90 days is estimated to be 20% to 30% based on the integration of the various data sources summarized here; the assumed placebo rate is 25% for this study.

The population sets used for analysis in this study are defined in Table 9.

TABLE 9
Analysis Populations

| Population | Description |
|---|---|
| Intent-to-Treat Set (ITT) | All participants randomized to study intervention. |
| | Participants are analyzed as randomized. |
| modified Intent-to-Treat Set | All randomized participants who receive their complete |
| (mITT) | weight-based dose of study intervention and undergo CPB |
| | procedure within 15 days of dosing. Participants are |
| | analyzed as randomized. |
| Per Protocol Set (PPS) | All participants in mITT and have no important protocol |
| | deviation. Participants are analyzed as randomized. All |
| | cases of prospectively defined protocol deviations are |
| | defined in the SAP prior to clinical database lock. |
| Post-operative Set (POS) | All participants in mITT and have stable pre-operative |
| | kidney function (prior to CPB [Visit 3] sCr remains <1.5x |
| | of baseline, with no KRT). Participants are analyzed as |
| | randomized. |
| Safety Set (SS) | All randomized participants who receive study intervention. |
| | Participants are analyzed according to the study intervention |
| | they actually receive. |
| PK Analysis Set (PKAS) | All ravulizumab-treated patients with at least 1 post- |
| | baseline PK concentration available. |
| PD Analysis Set (PDAS) | All randomized participants who received study |
| | intervention and at least 1 post dose PD measurement. |
| ADA Analysis Set (AAS) | All randomized participants who receive ravulizumab and |
| | have at least 1 reportable post- dose ADA result. |
| | Participants are analyzed according to the study intervention |
| | they actually receive. |

Abbreviations: ADA = antidrug antibody; CPB = cardiopulmonary bypass; KRT = kidney replacement therapy; PD = pharmacodynamic; PK = pharmacokinetic; SAP = statistical analysis plan; sCr = serum creatinine.

Evaluable PR, PD, and ADA data are defined as non-missing results generated from samples that comply with sample integrity requirements during sample collection, storage, shipment, and bioanalysis.

In general, descriptive statistics (n, mean, median, standard deviation, first and third quantiles, minimum, and maximum) are provided by treatment group and visit for each quantitative variable, and frequencies and percentages are provided by treatment group and visit for each qualitative variable. Graphical displays are provided as appropriate. Baseline is defined as Day 1 randomization prior to administration of study intervention, except where otherwise noted below. The final analysis and study unblinding occurs once the last enrolled participant completes Day 90 visit or early withdrawal. Analyses are performed using the SAS® software version 9.4 or higher.

The primary efficacy analysis is based on the Intent-to-Treat (ITT) Analysis Set. The primary endpoint is the MAKE event at 90 days post CPB (MAKE90), defined as meeting at least 1 of the following criteria: (1) decrease from baseline in eGFR of ≥25% at Day 90 post CPB. (2) occurrence of KRT through Day 90 post CPB, or (3) death from any cause through Day 90 post CPB. Baseline and post-baseline eGFRs are calculated from sCysC using the CKD-EPI formula. Baseline eGFR is based on the average of sCysC collected during screening and on Day 1 prior to study intervention administration.

The observed proportion of participants experiencing MAKE 90 are reported by treatment group. The treatment effect in the MAKE90 endpoint are estimated using the continuity-corrected Cochran-Mantel-Haenszel (CMH) method adjusting for the stratification variables. For missing endpoint of MAKE90 due to partial or complete missing of individual components, missing of KRT initiation or death is imputed as non-event, and missing of eGFR is handled by multiple imputation from mixed model repeated measures (MMRM) assuming missing at random by treatment group and then dichotomized into the binary variable. The MMRM model includes baseline, CKD stage, surgical type, visit, treatment group, and treatment group by visit interaction as fixed effects, and participant as a random effect, using all available data. Rubin's rule is used for combining results to yield multiple imputation point estimate and standard error. If the distribution of CMH test statistics strongly deviates from normality, the Wilson-Hilferty transformation is applied before combining using Rubin's rules. The p-value and 2-sided 95% confidence interval (CI) of the treatment difference in MAKE90 is reported.

A sensitivity analysis is conducted to evaluate the robustness of results against missing data estimation method. The missing data of MAKE90 is multiply imputed by treatment group based on logistic regression model. The logistic model includes CKD stage, surgical type, visit, treatment group, and treatment group by visit interaction as covariates. The CMH test along with Rubin's rule is applied in the same manner as main analysis. The Wilson-Hilferty transformation is applied if CMH test statistics strongly deviate from normality assumption.

A second sensitivity analysis is conducted based on multiple imputations from jump to reference (J2R) model. For participants who discontinue study without any further follow-up data, any missing values after discontinuation are imputed under the assumption that their outcome would be the similar to those in the placebo group with similar baseline characteristics. The CMH test is carried out in a similar manner to the main analysis.

In addition, the individual MAKE 90 components are analyzed. The proportion of participants with decrease in eGFR (CKD-EPI formula using sCysC)≥25% from baseline to Day 90 post CPB, or with initiation of KRT through Day 90 post CPB or death from any cause through Day 90 post CPB is summarized by treatment group and visit. The point estimate and associated 2-sided 95% CIs for treatment difference is presented by visit using CMH method, adjusting for stratification factors.

In addition to the primary treatment policy estimand, other supplementary estimands using the ITT population or other populations (i.e., modified Intent to Treat [mITT], Per Protocol Set [PPS] and Post-operative Set [POS]) is also defined to assess the robustness of the primary estimand effect. Details are provided in the SAP.

The secondary efficacy analyses are based on the ITT Analysis Set. A sequential testing procedure is performed for the key secondary endpoints in the following order:
A. CSA-AKI free at Day 90 post CPB defined as having no AKI within first 7 days post CPB or AKI recovery (stage 0) by Day 90 post CPB;
B. Free of severe CSA-AKI (KDIGO Stage 2 or 3) based on highest observed sCr within 7 days post CPB;
C. Free of any severe AKI (RIFLE Injury or Failure criteria) based on highest observed sCr within Day 30 post CPB;
D. Free of any severe AKI (KDIGO Stage 2 or 3) based on highest observed sCr within Day 30 post CPB; and
E. Free of any RIFLE Failure criteria based on highest observed sCr within Day 30 post CPB.
F. All-cause mortality from randomization through Day 90 post CPB

The highest observed sCr is from central laboratory samples collected at specified study visits from daily local laboratory sCr results at any time during the first 30 days. Baseline sCr for defining AKI are based on the average of sCr collected during screening and on Day 1 prior to study intervention administration. Specifically, when the null hypothesis for the primary endpoint is rejected, the key secondary endpoints are tested against the same significant level as primary endpoint, and the sequential testing is terminated when any null hypothesis fails to be rejected.

The analysis of key secondary endpoints is same as primary analysis. The observed proportions of participants meeting each key secondary endpoint are summarized by treatment group.

To estimate the treatment effect in key secondary endpoints, missing data due to early withdraw and all other events is imputed by last observation carried forward (LOCF) if applicable, otherwise the missing is imputed as not achieving the endpoint. The point estimates and associated 2-sided 95% CIs is estimated using the CMH method adjusting for stratification factors.

If the testing for primary endpoint shows statistical significance, the association between each of the key secondary endpoints 2 through 5 above and the following clinical outcomes are examined: death, initiation of KRT, readmission to hospital, duration (days) of KRT, duration (days) of ICU stay, duration (days) of ventilation, and duration (days) of hospitalization.

For binary clinical outcomes including death, initiation of KRT and readmission to hospital, the 2 by 2 contingency table of clinical outcome and key secondary endpoint is provided by treatment group and overall. Chi-square test is performed for testing the correlation between two variables.

For continuous clinical outcomes, the summary statistics are provided by key secondary endpoint category, by treatment group and overall. The linear regression is applied with key secondary endpoint and treatment group as covariates.

Similar to primary endpoint MAKE90, which is determined based on CKD-EPI formula using sCysC, MAKE90 is also determined based on CKD-EPI formula using sCr. In addition, MAKE30 and MAKE60 endpoints are determined similarly. The proportion of participants who experience MAKE at Days 30, 60, and 90 post CPB are summarized by treatment group and visit. The point estimate and associated p-values and 2 sided 95% CIs for treatment difference is presented using CMH method, adjusting for stratification factors.

In addition, the individual MAKE component is analyzed. The proportion of participants with decrease in eGFR≥25% from baseline to Days 30, 60, and 90 post CPB, or with initiation of KRT through Days 30, 60, and 90 post CPB or death from any cause through Days 30, 60, and 90 post CPB is summarized by treatment group and visit. The point estimate and associated 2-sided 95% CIs for treatment difference is presented by visit using CMH method, adjusting for stratification factors.

Additional analysis can be explored to examine the treatment effect for each individual component of MAKE at Day 30, Day 60, and Day 90 post CPB.

The proportion of participants with KRT or death occurred through Day 30, Day 60, and Day 90 post CPB is summarized by treatment group. The point estimates and associated 2-sided 95% CI are provided using the CMH method, adjusting for stratification factors.

The proportion of participants with CSA-AKI by highest stage observed within the first 3 and 7 days after CPB using modified KDIGO criteria is summarized by treatment group.

The proportion of participants with CSA-AKI free and free of any AKI is summarized at prespecified visit by treatment group and visit. The point estimates and associated 2-sided 95% CIs are estimated using the CMH method, adjusting for stratification factors.

The proportion of participants with AKI recovery (complete or partial), improving AKI, stable AK or progressing AKI at Days 15, 30, 60, and 90 post CPB amongst participants who experience CSA-AKI within 7 days post CPB is summarized by treatment group and visit. The point estimates and associated 2 sided 95% CIs are estimated, using the CMH method adjusting for stratification factors (if appropriate).

The duration (days) of index hospital and ICU stay is calculated based on date of discharge minus date of admission for each participant, where the index hospital and ICU stay refer to the hospital admission due to CPB operation. For patients on ventilators, the duration (days) on ventilator use is calculated based on date of releasing from ventilator minus date of starting ventilators. For patients on KRT, the duration (days) of KRT is calculated based on the last date on KRT minus date of KRT initiation.

The duration is summarized for each treatment group using descriptive statistics. It includes the number of observations, mean, standard deviation, median, minimum, maximum, interquartile range (IQR), first quartile, and third quartile values in each treatment group.

Hospital readmission rate (all-cause or AKI-related) through Day 30 and Day 90 post CPB is summarized by treatment group and visit. The point estimates and associated 2-sided 95% CIs is estimated, using the CMH method adjusting for stratification factors (if appropriate).

The following quality of life assessments are summarized by treatment group at baseline and each postbaseline time point using descriptive statistics for the observed value as well as the change from baseline: FACIT-Fatigue, EQ-5D-5L, and KDQOL-36.

The change from baseline is also summarized for each treatment group from the MMRM analysis; no formal treatment comparison is made. The point estimate and two-sided 95% CI for the mean difference in these measurement scores is presented.

Analyses of exploratory biomarkers data is described in a separate analysis plan and can be summarized after study completion.

A multiple testing procedure includes a test on the primary endpoint and then a pre-specified hierarchical test on the key secondary endpoints. Statistical significance is assessed in the order of the hypotheses. The secondary endpoints are tested in the fixed sequence only if the primary endpoint is statistically significant. The testing of key secondary endpoints continues down the hierarchy only if the test for the preceding secondary endpoint is statistically significant.

At the second interim analysis. i.e., approximately 50% participants complete the Primary Evaluation Period, the sample size re-estimation is conducted. The conditional power based on the observed treatment effect is computed to determine if the enrollment continues to achieve planned or increased sample size. The primary endpoint is tested in the final analysis when all enrolled participants complete the Primary Evaluation Period under a one-sided significance level of 0.025. If the test of the primary endpoint is statistically significant at the final analysis, statistical significance for the key secondary endpoints is assessed in a fixed sequence.

Under this pre-specified multiple testing procedure and hierarchical testing strategy, the overall type-I error is controlled at a one-sided 0.025 level for multiplicity across primary and key secondary endpoints. The other planned interim analysis at 30% sample size for early futility assessment has no risk of type-I error inflation.

The safety is assessed based on AEs, clinical laboratory findings, and vital sign findings. All safety analyses are performed on the Safety Set.

The incidence of treatment-emergent adverse events (TEAEs), TEAEs leading to withdrawal from the study, and treatment-emergent serious adverse events (TESAEs) is summarized by treatment group. TEAEs are defined as those AEs with onset or existing events that worsen in severity from the initiation of dosing of study intervention through 90 days after the dose of study intervention. All AEs are coded using the Medical Dictionary for Regulatory Activities (MedDRA), version 24.1 or higher, and are summarized by System Organ Class (SOC) and Preferred Term overall, by severity, and by relationship to study intervention. Detailed by—participant listings of TEAEs, TESAEs, related TEAEs, and TEAEs leading to withdrawal from the study is provided. Participants having multiple AEs within a category (e.g., overall, SOC. Preferred Term) are counted once in that category. For severity tables, a participant's most severe event within a category is counted. AESIs (such as meningococcal infections) are analyzed similarly.

Abnormal physical examination findings are classified as AEs and analyzed accordingly.

Vital signs are summarized descriptively by treatment group at baseline and post-baseline time points and for changes from baseline.

Observed values and changes from baseline in clinical chemistry, hematology, and urinalysis are summarized descriptively by treatment group at baseline and at each post-baseline time point. For laboratory results that can be classified as normal, low, or high based on normal range values, shifts from baseline in classification are summarized for all study visits.

The PK and PD analyses include all data in the PK Analysis Set and PD Analysis Set, respectively.

Graphs of mean serum ravulizumab concentration-time profiles are constructed. Graphs of serum concentration-time profiles for individual participants can also be provided. Descriptive statistics are calculated for serum concentration data at each sampling time, as appropriate.

The PD effects of ravulizumab are evaluated by assessing the absolute values and changes and percentage changes from baseline in serum free C5 concentrations over time, as appropriate. Descriptive statistics are calculated for the PD data at each sampling time, as appropriate.

All antidrug antibody (ADA) analyses are performed on the ADA Analysis Set (AAS).

The incidence of ADA response categories is summarized as absolute occurrence (n) and percent of all participants (%) in the ravulizumab group at Days 30 and 90. Confirmed antibody positive samples is further evaluated for antibody titer and the presence of neutralizing antibodies. Maximum ADA titer levels are listed and summarized for ADA positive participants as absolute occurrence (n) and percent (%) of all participants.

Association of immunogenicity variable with impact on drug exposure, efficacy and safety can be explored.

Subgroup analyses of the primary endpoint and key secondary endpoints are performed for the following subset of participants: (1) participants with different CKD stage at baseline, (2) participants with different surgical types during CPB, (3) participants in age groups 18 to 60, 61 to 75, and >75 years, (4) participants with body weight groups at baseline of ≥30-59 kg, 60-99 kg, ≥100 kg, (5) participants with the following levels of albuminuria (measured as albumin to creatinine ratio [ACR]) at baseline (KDIGO 2013):<30 mg albumin/g creatinine, ≥30 to <300 mg albumin/g creatinine, ≥300 mg albumin/g creatinine, and (6) participants with and without diabetes mellitus.

Two interim analyses performed by an independent data monitoring committee (DMC) are planned after approximately 30% (~220 participants) and 50% (~368 participants) of the randomized participants have completed the Primary Evaluation Period (i.e., Day 90 visit post CPB). The purpose of the first interim analysis is to assess early stopping for futility and the second interim analysis is planned to conduct sample size re-estimation.

For futility and sample size re-estimation assessment, conditional power for the primary endpoint analysis is computed at the interim analyses using the observed trend. If the conditional power is less than 20% in the first interim analysis, the study is considered for early stopping for futility. However, the futility criterion is non-binding. In other words, if the primary endpoint meets the prespecified futility criteria at the first interim analysis, the study may continue without stopping the study for futility.

For the sample size re-estimation at the second interim analysis, if the conditional power falls in the promising zone (0.5, 0.9), the study sample size is increased. This promising zone is chosen to ensure that the conventional analysis at the end of the study does not inflate the study type-I error under the planned maximum sample size increase. To prevent the potential unblinding of the interim analysis result in the sample size re-estimation procedure, a step function is utilized to guide the sample size increase if the interim analysis result falls in the pre-specified promising zone. Details of the interim analysis are documented in the interim statistical analysis plan (iSAP) prior to the interim analysis.

### 12. Clinical Laboratory Tests

The tests detailed in Table 10 are performed by the study central laboratory unless otherwise noted. Local laboratory results are only required in the event that the central laboratory results are not available in time for eligibility, study intervention administration and/or response evaluation. If a local sample is required, it is important that the sample for central analysis is obtained at the same time. Additionally, if the local laboratory results are used to make an eligibility or a study intervention decision or response evaluation, the results must be available in the participant's source documents. Serum creatinine tests done locally as standard of care in the first 30 days post CBP are reviewed. The highest observed result is reported on the CRF. Additional laboratory tests can be performed at any time during the study as determined necessary by the Investigator or required by local regulations. WOCBP are only enrolled after a negative serum pregnancy test result at Screening. Additional pregnancy testing is performed per the time points specified in the Schedule of Assessments.

TABLE 10
Protocol-Required Laboratory Assessments

| Laboratory | |
|---|---|
| Assessments | Parameters |
| Hematology | Hemoglobin |
| | Hematocrit |
| | Platelet count |
| | RBC count |
| | RBC indices: |
| | Mean corpuscular volume |
| | Distribution width |
| | WBC count with differential: |
| | Neutrophils |
| | Lymphocytes |
| | Monocytes |
| | Eosinophils |
| | Basophils |
| Clinical chemistry | Alanine aminotransferase |
| | Albumin |
| | Alkaline phosphatase |
| | Aspartate aminotransferase |
| | Bicarbonate |
| | Blood urea nitrogen |
| | Calcium |
| | Chloride |
| | Creatinine (sCr) |
| | Cystatin C (sCysC) |
| | Glucose, nonfasting |
| | Potassium |
| | Sodium |
| | Total and direct bilirubin |
| | Total protein |
| | High-sensitivity cardiac Troponin T (hs-cTnT) |
| | Uric acid |
| Urine chemistry | Urinary albumin concentration |
| | Urinary creatinine concentration |
| | Albumin/creatinine ratio (UACR) |
| PK/PD | Serum PK |
| | Serum PD (free C5) |
| Immunogenicity | ADA and NAb |
| Exploratory | Assessments can include, but are not limited to, biomarkers of |
| Biomarkers (blood and | complement activation (e.g., soluble C5b-9), endothelial damage |
| urine) | (e.g., plasma thrombomodulin [TM]), vascular inflammation |
| | (e.g., TNF-RI), cell-cycle arrest (TIMP-2), and renal injury (e.g., |
| | neutrophil gelatinase-associated lipocalin [NGAL]). |
| Other tests | Serum follicle-stimulating hormone and estradiol (as needed |
| | to confirm WOCBP status) |
| | Serum human chorionic gonadotropin pregnancy test (as |
| | needed for WOCBP) |

Abbreviations:
ADA = antidrug antibody;
Ba = complement factor B;
C5 = complement component 5;
NAb = neutralizing antibodies;
PD = pharmacodynamic;
PK = pharmacokinetic;
RBC = red blood cell;
WBC = white blood cell;
WOCBP = women of childbearing potential

### 13. Exploratory Biomarkers

Blood and urine samples are collected for exploratory assessments related to ravulizumab or CSA-AKI and related diseases. These samples can also be used for further exploratory development of assays related to the mechanism of action of ravulizumab, disease process and/or pathways associated with CSA-AKI disease state. The results of biomarker analyses can be reported in the CSR or later in a separate study summary.

### 14. COVID-19 Vaccine Risk Assessment

There is currently no information available evaluating the safety and efficacy of COVID-19 vaccines in participants treated with ravulizumab. It is unlikely that the immune response to a COVID 19 vaccine (and therefore the efficacy of the vaccination) would be diminished by ravulizumab administration, based on ravulizumab's mechanism of action. It is also unlikely that COVID-19 vaccination would impact ravulizumab's mechanism of action.

Vaccination may further activate complement. As a result, participants with complement mediated diseases may experience increased signs and symptoms of their underlying disease. Therefore, participants should be closely monitored for disease symptoms after recommended vaccination. Because vaccines may activate complement, if possible, consider vaccination when the underlying complement mediated disease is clinically controlled and when systemic C5 inhibitor concentration (and subsequent complement blockade) is relatively high, shortly after administration. Local and national guidelines should be consulted for recommendations related to COVID-19 vaccination.

The potential risks identified, and mitigation measures put in place in light of the COVID-19 vaccination rollout are provided in Table 11.

TABLE 11
Potential Operational Risks and Mitigation Measures due to COVID-19 Vaccine

| | | |
|---|---|---|
| Risks Category | Summary of Data/Rationale for Risk | Mitigation Strategy |

Potential risks

| | | |
|---|---|---|
| Data quality and | Missing data due to appointments for | Capture specific |
| integrity | COVID-19 vaccination or side effects | information in the CRF that |
| | of COVID-19 vaccine may impact | explains the reason for |
| | study visit schedules and increase | missing data (e.g., missed |
| | missed visits and/or participant study | study visits due to |
| | discontinuations, inadvertently resulting | appointments for |
| | in missing data (e.g., for protocol- | COVID-19 vaccination or |
| | specified procedures). | side effects of COVID-19 |
| | | vaccine). |

Abbreviations: COVID-19 = coronavirus disease 2019; CRF = case report form.

SEQUENCE SUMMARY

SEQ ID NO: 1
GYIFSNYWIQ
SEQ ID NO: 2
EILPGSGSTEYTENFKD
SEQ ID NO: 3
YFFGSSPNWYFDV
SEQ ID NO: 4
GASENIYGALN
SEQ ID NO: 5
GATNLAD
SEQ ID NO: 6
QNVLNTPLT
SEQ ID NO: 7
QVQLVQSGAE VKKPGASVKV SCKASGYIFS NYWIQWVRQA PGQGLEWMGE
ILPGSGSTEY TENFKDRVTM TRDTSTSTVY MELSSLRSED TAVYYCARYF
FGSSPNWYFD VWGQGTLVTV SS
SEQ ID NO: 8
DIQMTQSPSS LSASVGDRVT ITCGASENIY GALNWYQQKP GKAPKLLIYG
ATNLADGVPS RFSGSGSGTD FTLTISSLQP EDFATYYCQN VLNTPLTFGQ
GTKVEIK
SEQ ID NO: 9
ASTKGPSVFP LAPCSRSTSE STAALGCLVK DYFPEPVTVS WNSGALTSGV
HTFPAVLQSS GLYSLSSVVT VPSSNEGTQT YTCNVDHKPS NTKVDKTVER
KCCVECPPCP APPVAGPSVF LFPPKPKDTL MISRTPEVTC VVVDVSQEDP
EVQFNWYVDG VEVHNAKTKP REEQFNSTYR VVSVLTVLHQ DWLNGKEYKC
KVSNKGLPSS IEKTISKAKG QPREPQVYTL PPSQEEMTKN QVSLTCLVKG
FYPSDIAVEW ESNGQPENNY KTTPPVLDSD GSFFLYSRLT VDKSRWQEGN
VFSCSVMHEA LHNHYTQKSL SLSLGK
SEQ ID NO: 10
QVQLVQSGAE VKKPGASVKV SCKASGYIFS NYWIQWVRQA PGQGLEWMGE
ILPGSGSTEY TENFKDRVTM TRDTSTSTVY MELSSLRSED TAVYYCARYF
FGSSPNWYFD VWGQGTLVTV SSASTKGPSV FPLAPCSRST SESTAALGCL
VKDYFPEPVT VSWNSGALTS GVHTFPAVLQ SSGLYSLSSV VTVPSSNFGT
QTYTCNVDHK PSNTKVDKTV ERKCCVECPP CPAPPVAGPS VFLFPPKPKD
TLMISRTPEV TCVVVDVSQE DPEVQFNWYV DGVEVHNAKT KPREEQENST
YRVVSVLTVL HQDWLNGKEY KCKVSNKGLP SSIEKTISKA KGQPREPQVY
TLPPSQEEMT NQVSLTCLVK GFYPSDIAVE WESNGQPENN YKTTPPVLDS
DGSFFLYSRL TVDKSRWQEG NVFSCSVMHE ALHNHYTQKS LSLSLGK
SEQ ID NO: 11
DIQMTQSPSS LSASVGDRVT ITCGASENIY GALNWYQQKP GKAPKLLIYG
ATNLADGVPS RFSGSGSGTD FTLTISSLQP EDFATYYCQN VLNTPLTEGQ
GTKVEIKRTV AAPSVFIFPP SDEQLKSGTA SVVCLLNNFY PREAKVQWKV
DNALQSGNSQ ESVTEQDSKD STYSLSSTLT LSKADYEKHK VYACEVTHQG
LSSPVTKSFN RGEC
SEQ ID NO: 12
QVQLVQSGAE VKKPGASVKV SCKASGHIFS NYWIQWVRQA PGQGLEWMGE
ILPGSGHTEY TENFKDRVTM TRDTSTSTVY MELSSLRSED TAVYYCARYF
FGSSPNWYFD VWGQGTLVTV SS
SEQ ID NO: 13
ASTKGPSVFP LAPCSRSTSE STAALGCLVK DYFPEPVTVS WNSGALTSGV
HTFPAVLQSS GLYSLSSVVT VPSSNFGTQT YTCNVDHKPS NTKVDKTVER
KCCVECPPCP APPVAGPSVF LFPPKPKDTL MISRTPEVTC VVVDVSQEDP
EVQFNWYVDG VEVHNAKTKP REEQFNSTYR VVSVLTVLHQ DWLNGKEYKC
KVSNKGLPSS IEKTISKAKG QPREPQVYTL PPSQEEMTKN QVSLTCLVKG
FYPSDIAVEW ESNGQPENNY KTTPPVLDSD GSFFLYSRLT VDKSRWQEGN
VFSCSVLHEA LHSHYTQKSL SLSLGK
SEQ ID NO: 14
QVQLVQSGAE VKKPGASVKV SCKASGHIFS NYWIQWVRQA PGQGLEWMGE
ILPGSGHTEY TENFKDRVTM TRDTSTSTVY MELSSLRSED TAVYYCARYF
FGSSPNWYFD VWGQGTLVTV SSASTKGPSV FPLAPCSRST SESTAALGCL
VKDYFPEPVT VSWNSGALTS GVHTFPAVLQ SSGLYSLSSV VTVPSSNFGT
QTYTCNVDHK PSNTKVDKTV ERKCCVECPP CPAPPVAGPS VFLFPPKPKD
TLMISRTPEV TCVVVDVSQE DPEVQFNWYV DGVEVHNAKT KPREEQFNST
YRVVSVLTVL HQDWLNGKEY KCKVSNKGLP SSIEKTISKA KGQPREPQVY
TLPPSQEEMT KNQVSLTCLV KGFYPSDIAV EWESNGQPEN NYKTTPPVLD
SDGSFFLYSR LTVDKSRWQE GNVFSCSVLH EALHSHYTQK SLSLSLGK
SEQ ID NO: 15
ASTKGPSVFP LAPCSRSTSE STAALGCLVK DYFPEPVTVS WNSGALTSGV
HTFPAVLQSS GLYSLSSVVT VTSSNEGTQT YTCNVDHKPS NTKVDKTVER
KCCVECPPCP APPVAGPSVF LFPPKPKDTL YITREPEVTC VVVDVSHEDP
EVQFNWYVDG MEVHNAKTKP REEQFNSTER VVSVLTVVHQ DWLNGKEYKC
KVSNKGLPAP IEKTISKTKG QPREPQVYTL PPSREEMTKN QVSLTCLVKG
FYPSDIAVEW ESNGQPENNY KTTPPMLDSD GSFFLYSKLT VDKSRWQQGN
VFSCSVMHEA LHNHYTQKSL SLSPGK
SEQ ID NO: 16
QVQLVQSGAE VKKPGASVKV SCKASGYIFS NYWIQWVRQA PGQGLEWMGE
ILPGSGSTEY TENFKDRVTM TRDTSTSTVY MELSSLRSED TAVYYCARYF
FGSSPNWYFD VWGQGTLVTV SSASTKGPSV FPLAPCSRST SESTAALGCL
VKDYFPEPVT VSWNSGALTS GVHTFPAVLQ SSGLYSLSSV VTVTSSNFGT
QTYTCNVDHK PSNTKVDKTV ERKCCVECPP CPAPPVAGPS VFLFPPKPKD
TLYITREPEV TCVVVDVSHE DPEVQFNWYV DGMEVHNAKT KPREEQENST
FRVVSVLTVV HQDWLNGKEY KCKVSNKGLP APIEKTISKT KGQPREPQVY
TLPPSREEMT KNQVSLTCLV KGFYPSDIAV EWESNGQPEN NYKTTPPMLD
SDGSFFLYSK LTVDKSRWQQ GNVFSCSVMH EALHNHYTQK SLSLSPGK
SEQ ID NO: 17
GASENIYHALN
SEQ ID NO: 18
EILPGSGHTEYTENFKD
SEQ ID NO: 19
GHIFSNYWIQ
SEQ ID NO: 20
QVQLVQSGAE VKKPGASVKV SCKASGHIFS NYWIQWVRQA PGQGLEWMGE
ILPGSGHTEY TENFKDRVTM TRDTSTSTVY MELSSLRSED TAVYYCARYF
FGSSPNWYFD VWGQGTLVTV SSASTKGPSV FPLAPCSRST SESTAALGCL
VKDYFPEPVT VSWNSGALTS GVHTFPAVLQ SSGLYSLSSV VTVPSSNFGT
QTYTCNVDHK PSNTKVDKTV ERKCCVECPP CPAPPVAGPS VFLFPPKPKD
TLMISRTPEV TCVVVDVSQE DPEVQFNWYV DGVEVHNAKT KPREEQENST
YRVVSVLTVL HQDWLNGKEY KCKVSNKGLP SSIEKTISKA KGQPREPQVY
TLPPSQEEMT KNQVSLTCLV KGFYPSDIAV EWESNGQPEN NYKTTPPVLD
SDGSFFLYSR LTVDKSRWQE GNVFSCSVMH EALHNHYTQK SLSLSLGK
SEQ ID NO: 21
SYAIS
SEQ ID NO: 22
GIGPFFGTANYAQKFQG
SEQ ID NO: 23
DTPYFDY
SEQ ID NO: 24
SGDSIPNYYVY
SEQ ID NO: 25
DDSNRPS
SEQ ID NO: 26
QSFDSSLNAEV
SEQ ID NO: 27
QVQLVQSGAE VKKPGSSVKV SCKASGGTFS SYAISVWRQA PGQGLEWMGG
IGPFFGTANY AQKFQGRVTI TADESTSTAY MELSSLRSED TAVYYCARDT
PYFDYWGQGT LVTVSS
SEQ ID NO: 28
DIELTQPPSV SVAPGQTARI SCSGDSIPNY YVYWYQQKPG QAPVLVIYDD
SNRPSGIPER FSGSNSGNTA TLTISGTQAE DEADYYCQSF DSSLNAEVFG
GGTKLTVL
SEQ ID NO: 29
NYIS
SEQ ID NO: 30
IIDPDDSYTEYSPSFOG
SEQ ID NO: 31
YEYGGFDI
SEQ ID NO: 32
SGDNIGNSYVH
SEQ ID NO: 33
KDNDRPS
SEQ ID NO: 34
GTYDIESYV
SEQ ID NO: 35
EVQLVQSGAE VKKPGESLKI SCKGSGYSFT NYISWVRQMP GKGLEWMGII
DPDDSYTEYS PSFQGQVTIS ADKSISTAYL QWSSLKASDT AMYYCARYEY
GGFDIWGQGT LVTVSS
SEQ ID NO: 36
SYELTQPPSV SVAPGQTARI SCSGDNIGNS YVHWYQQKPG QAPVLVIYKD
NDRPSGIPER FSGSNSGNTA TLTISGTQAE DEADYYCGTY DIESYVFGGG
TKLTVL
SEQ ID NO: 37
SSYYVA
SEQ ID NO: 38
AIYTGSGATYKASWAKG
SEQ ID NO: 39
DGGYDYPTHAMHY
SEQ ID NO: 40
QASQNIGSSLA
SEQ ID NO: 41
GASKTHS
SEQ ID NO: 42
QSTKVGSSYGNH
SEQ ID NO: 43
QVQLVESGGG LVQPGGSLRL SCAASGFTSH SSYYVAWVRQ APGKGLEWVG
AIYTGSGATY KASWAKGRFT ISKDTSKNQV VLTMTNMDPV DTATYYCASD
GGYDYPTHAM HYWGQGTLVT VSS
SEQ ID NO: 44
DVVMTQSPSS LSASVGDRVT ITCQASQNIG SSLAWYQQKP GQAPRLLIYG
ASKTHSGVPS RFSGSGSGTD FTLTISSLQP EDVATYYCQS TKVGSSYGNH
FGGGTKVEIK
SEQ ID NO: 45
QVQLVESGGG LVQPGRSLRL SCAASGFTVH SSYYMAWVRQ APGKGLEWVG
AIFTGSGAEY KAEWAKGRVT ISKDTSKNQV VLTMTNMDPV DTATYYCASD
AGYDYPTHAM HYWGQGTLVT VSSASTKGPS VFPLAPSSKS TSGGTAALGC
LVKDYFPEPV TVSWNSGALT SGVHTFPAVL QSSGLYSLSS VVTVPSSSLG
TQTYICNVNH KPSNTKVDKK VEPKSCDKTH TCPPCPAPEL RRGPKVFLFP
PKPKDTLMIS RTPEVTCVVV DVSHEDPEVK FNWYVDGVEV HNAKTKPREE
QYNSTYRVVS VLTVLHQDWL NGKEYKCKVS NKGLPSSIEK TISKAKGQPR
EPQVYTLPPS REEMTKNQVS LTCLVKGFYP SDIAVEWESN GQPENNYKTT
PPVLDSDGSF FLYSKLTVDK SRWQQGNVFS CSVLHEALHA HYTRKELSLS
P
SEQ ID NO: 46
DIQMTQSPSS LSASVGDRVT ITCRASQGIS SSLAWYQQKP GKAPKLLIYG
ASETESGVPS RFSGSGSGTD FTLTISSLQP EDFATYYCQN TKVGSSYGNT
FGGGTKVEIK RTVAAPSVFI FPPSDEQLKS GTASVVCLLN NFYPREAKVQ
WKVDNALQSG NSQESVTEQD SKDSTYSLSS TLTLSKADYE KHKVYACEVT
HQGLSSPVTK SFNRGEC
SEQ ID NO: 47
QVQLQESGPGLVKPSETLSLTCTVSGDSVSSSYWTWIRQPPGKGLEWIGYIYYSGSSN
YNPSLKSRATISVDTSKNQFSLKLSSVTAADTAVYYCAREGNVDTTMIFDYWGQGTLV
TVSS
SEQ ID NO: 48
AIQMTQSPSSLSASVGDRVTITCRASQGIRNDLGWYQQKPGKAPKLLIYAASSLQSGVP
SRFAGRGSGTDFTLTISSLQPEDFATYYCLQDFNYPWTFGQGTKVEIK
SEQ ID NO: 49
QVQLQESGPGLVKPSETLSLTCTVSGDSVSSSYWTWIRQPPGKGLEWIGYIYYSGSSNY
NPSLKSRATISVDTSKNQFSLKLSSVTAADTAVYYCAREGNVDTTMIFDYWGQGTLVTV
SSASTKGPSVFPLAPCSRSTSESTAALGCLVKDYFPEPVTVSWNSGALTSGVHTFPAVL
QSSGLYSLSSVVTVPSSSLGTKTYTCNVDHKPSNTKVDKRVESKYGPPCPPCPAPEFLG
GPSVFLFPPKPKDTLMISRTPEVTCVVVDVSQEDPEVQFNWYVDGVEVHNAKTKPREEQ
FNSTYRVVSVLTVLHQDWLNGKEYKCKVSNKGLPSSIEKTISKAKGQPREPQVYTLPPS
QEEMTKNQVSLTCLVKGFYPSDIAVEWESNGQPENNYKTTPPVLDSDGSFFLYSRLTVD
KSRWQEGNVFSCSVMHEALHNHYTQKSLSLSLGK
SEQ ID NO: 50
AIQMTQSPSSLSASVGDRVTITCRASQGIRNDLGWYQQKPGKAPKLLIYAASSLQSGVP
SRFAGRGSGTDFTLTISSLQPEDFATYYCLQDFNYPWTFGQGTKVEIKRTVAAPSVFIF
PPSDEQLKSGTASVVCLLNNFYPREAKVQWKVDNALQSGNSQESVTEQDSKDSTYSLSS
TLTLSKADYEKHKVYACEVTHQGLSSPVTKSFNRGEC

1. A method of preparing a human patient with chronic kidney disease (CKD) for cardiac surgery with cardiopulmonary bypass (CPB), wherein the method comprises administering to the patient an effective amount of an anti C5 antibody, or antigen binding fragment thereof, comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, wherein the anti C5 antibody, or antigen binding fragment thereof, is administered prior to the surgery once at a dose of:
a) 2700 mg to a patient weighing ≥30 to <40 kg;
b) 3000 mg to a patient weighing ≥40 to <60 kg;
c) 3300 mg to a patient weighing ≥60 to <100 kg; or
d) 3600 mg to a patient weighing ≥100 kg.

2. A method of inhibiting terminal complement activation in a human patient with CKD prior to cardiac surgery with CPB, wherein the method comprises administering to the patient an effective amount of an anti C5 antibody, or antigen binding fragment thereof, comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, wherein the anti C5 antibody, or antigen binding fragment thereof, is administered prior to the surgery once at a dose of:
a) 2700 mg to a patient weighing ≥30 to <40 kg;
b) 3000 mg to a patient weighing ≥40 to <60 kg;
c) 3300 mg to a patient weighing ≥60 to <100 kg; or
d) 3600 mg to a patient weighing ≥100 kg.

3. A method of treating a human patient with CKD prior to cardiac surgery with CPB, wherein the method comprises administering to the patient an effective amount of an anti-C5 antibody, or antigen binding fragment thereof, comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, wherein the anti-C5 antibody, or antigen binding fragment thereof, is administered prior to the surgery once at a dose of:
a) 2700 mg to a patient weighing ≥30 to <40 kg;
b) 3000 mg to a patient weighing ≥40 to <60 kg;
c) 3300 mg to a patient weighing ≥60 to <100 kg; or
d) 3600 mg to a patient weighing ≥100 kg.

4. A method of preventing or reducing cardiac surgery associated acute kidney injury (CSA-AKI) in a human patient with CKD, wherein the method comprises administering to the patient an effective amount of an anti-C5 antibody, or antigen binding fragment thereof,
wherein the anti-C5 antibody, or antigen binding fragment thereof, comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, and
wherein the anti-C5 antibody, or antigen binding fragment thereof, is administered prior to cardiac surgery with CPB once at a dose of:
a) 2700 mg to a patient weighing ≥30 to <40 kg;
b) 3000 mg to a patient weighing ≥40 to <60 kg;
c) 3300 mg to a patient weighing ≥60 to <100 kg; or
d) 3600 mg to a patient weighing ≥100 kg.

5. A method of preventing or reducing one or more major adverse kidney events (MAKE) in a human patient with CKD, wherein the method comprises administering to the patient an effective amount of an anti-C5 antibody, or antigen binding fragment thereof,
wherein the anti-C5 antibody, or antigen binding fragment thereof, comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, and
wherein the anti-C5 antibody, or antigen binding fragment thereof, is administered prior to cardiac surgery with CPB, once at a dose of:
a) 2700 mg to a patient weighing ≥30 to <40 kg;
b) 3000 mg to a patient weighing ≥40 to <60 kg;
c) 3300 mg to a patient weighing ≥60 to <100 kg; or
d) 3600 mg to a patient weighing ≥100 kg.

6. The method of claim 1, wherein the anti-C5 antibody, or antigen binding fragment thereof, is administered at least one calendar day prior to the CPB.

7. The method of claim 1, wherein the anti-C5 antibody, or antigen binding fragment thereof, is administered from one to seven calendar days prior to the CPB.

8. The method of claim 1, wherein the anti-C5 antibody, or antigen binding fragment thereof, further comprises a variant human Fc constant region that binds to human neonatal Fc receptor (FcRn), wherein the variant human Fc constant region comprises Met429Leu and Asn435Ser substitutions at residues corresponding to methionine 428 and asparagine 434 of a native human IgG Fc constant region, each in EU numbering.

9. The method of claim 1, wherein the anti-C5 antibody comprises a heavy chain variable region set forth in SEQ ID NO:12 and a light chain variable region set forth in SEQ ID NO:8.

10. The method of claim 1, wherein the anti-C5 antibody further comprises a heavy chain constant region set forth in SEQ ID NO:13.

11. The method of claim 1, wherein the antibody comprises a heavy chain polypeptide comprising the amino acid sequence set forth in SEQ ID NO:14 and a light chain polypeptide comprising the amino acid sequence set forth in SEQ ID NO:11.

12.-19. (canceled)

20. The method of claim 1, wherein the anti-C5 antibody, or antigen binding fragment thereof, is formulated for intravenous administration.

21. The method of claim 1, wherein the cardiac surgery is selected from the group consisting of coronary artery bypass graft (CABG), valve replacement or repair, insertion of a pacemaker or an implantable cardioverter defibrillator (ICD), Maze surgery, Maze surgery, heart transplant, and insertion of a ventricular assist device (VAD) or total artificial heart (TAH), and transcatheter structural heart surgery.

22. The method of claim 1, wherein the single pre-operative weight-based dose of the anti-C5 antibody, or antigen binding fragment thereof, results in at least 18 days of complete C5 inhibition.

23. The method of claim 1, wherein the method:
a) prevents the need for kidney replacement therapy (KRT);
b) prevents or reduces CSA-AKI in the human patient with CKD;
c) results in stable CSA-AKI within 7, 30, 45, 60, or 90 days post surgery, characterized by sCr≥2.0−<3.0×baseline;
d) results in an improvement from CSA-AKI within 7, 30, 45, 60, or 90 days post surgery, wherein the improvement is characterized by sCr≥1.5−<2.0×baseline;
e) results in a partial recovery from CSA-AKI within 7, 30, 45, 60, or 90 days post surgery, wherein the partial recovery is characterized by sCr≥1.1−<1.5×baseline;
f) results in a complete recovery from CSA-AKI within 7, 30, 45, 60, or 90 days post surgery, wherein the complete recovery is characterized by sCr<1.1×baseline;
g) prevents or reduces one or more MAKE in the human patient with CKD;
h) results in a change from baseline in quality of life as assessed via a Quality of Life Assessment; and/or
i) results in a shift toward normal levels of biomarkers associated with vascular inflammation (e.g., shed tumor necrosis factor receptor 1 [TNF-R1 or sTNF-R1]), endothelial damage and/or activation (e.g., thrombomodulin), renal injury (e.g., neutrophil gelatinase-associated lipocalin [NGAL]), inducers of cell-cycle arrest (e.g., tissue inhibitor of metalloproteinase-2 [TIMP-2]), and/or complement proteins and complement activation pathway products (e.g., soluble C5b-9).

24. (canceled)

25. The method of claim 1, wherein the human patient comprises a patient with cardiac surgery associated acute kidney injury (CSA-AKI), wherein CSA-AKI is characterized by an increase in:
a) serum creatinine (sCr) or serum Cystatin C (sCysC) by ≥0.3 mg/dL in a 48-hour period within 7 days following CPB and/or
b) sCr or or sCysC≥1.5 times baseline within 7 days following CPB or at Day 15, 30, 60 or 90 post CPB.

26. (canceled)

27. The method of claim 1, wherein the human patient is free of severe CSA-AKI based on highest observed sCr, within 7, 30, 45, 60, or 90 days post CPB, as assessed by Modified “Risk, Injury, Failure, Loss of kidney function, and End-stage kidney disease” (RIFLE) Criteria and/or Kidney Disease Improving Global Outcomes (KDIGO) criteria.

28.-32. (canceled)

33. The method of claim 23, wherein the one or more MAKE is selected from the group consisting of:
a) sustained kidney dysfunction (SKD) defined as an estimated glomerular filtration rate (eGFR)>25% below baseline post CPB,
b) occurrence of kidney replacement therapy (KRT) post CPB, and
c) death from any cause post CPB.

34. The method of claim 33, wherein the decrease in eGFR is determined by the Chronic Kidney Disease Epidemiology Collaboration (CKD-EPI) formula based on serum Cystatin C (sCysC) or serum creatinine (sCr).

35. (canceled)

36. The method of claim 23, wherein the Quality of Life Assessment is a Kidney Disease Quality of Life instrument (KDQOL-36), The European Quality of Life Group's 5 dimension 5-level (EQ-5D-5L), or The Functional Assessment of Chronic Illness Therapy (FACIT) Fatigue scale.

37. (canceled)

38. A kit comprising:
(a) a dose of an anti-C5 antibody, or antigen binding fragment thereof, comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively; and
(b) instructions for using the anti-C5 antibody, or antigen binding fragment thereof, in the method of claim 1.

39.-48. (canceled)

49. The anti-C5 antibody, or antigen-binding fragment thereof, of claim 1, wherein the anti-C5 antibody, or antigen binding fragment thereof, is ravulizumab (ULTOMIRIS®).

50. (canceled)


SEQUENCE LISTING (50 sequences)

SEQ ID NO: 1 - protein, 10 aa, synthetic construct
GYIFSNYWIQ

SEQ ID NO: 2 - protein, 17 aa, synthetic construct
EILPGSGSTEYTENFKD

SEQ ID NO: 3 - protein, 13 aa, synthetic construct
YFFGSSPNWYFDV

SEQ ID NO: 4 - protein, 11 aa, synthetic construct
GASENIYGALN

SEQ ID NO: 5 - protein, 7 aa, synthetic construct
GATNLAD

SEQ ID NO: 6 - protein, 9 aa, synthetic construct
QNVLNTPLT

SEQ ID NO: 7 - protein, 122 aa, synthetic construct [sequence omitted]

SEQ ID NO: 8 - protein, 107 aa, synthetic construct [sequence omitted]

SEQ ID NO: 9 - protein, 326 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 447 aa, synthetic construct
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 214 aa, synthetic construct [sequence omitted]

SEQ ID NO: 12 - protein, 122 aa, synthetic construct [sequence omitted]

SEQ ID NO: 13 - protein, 326 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 14 - protein, 448 aa, synthetic construct
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 15 - protein, 326 aa, synthetic construct
ASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLYITREPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 16 - protein, 448 aa, synthetic construct
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLYITREPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 17 - protein, 11 aa, synthetic construct
GASENIYHALN

SEQ ID NO: 18 - protein, 17 aa, synthetic construct
EILPGSGHTEYTENFKD

SEQ ID NO: 19 - protein, 10 aa, synthetic construct
GHIFSNYWIQ

SEQ ID NO: 20 - protein, 448 aa, synthetic construct
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 5 aa, synthetic construct
SYAIS

SEQ ID NO: 22 - protein, 17 aa, synthetic construct
GIGPFFGTANYAQKFQG

SEQ ID NO: 23 - protein, 7 aa, synthetic construct
DTPYFDY

SEQ ID NO: 24 - protein, 11 aa, synthetic construct
SGDSIPNYYVY

SEQ ID NO: 25 - protein, 7 aa, synthetic construct
DDSNRPS

SEQ ID NO: 26 - protein, 11 aa, synthetic construct
QSFDSSLNAEV

SEQ ID NO: 27 - protein, 116 aa, synthetic construct
QVQLVQSGAEVKKPGSSVKVSCKASGGTFSSYAISVWRQAPGQGLEWMGGIGPFFGTANYAQKFQGRVTITADESTSTAYMELSSLRSEDTAVYYCARDTPYFDYWGQGTLVTVSS

SEQ ID NO: 28 - protein, 108 aa, synthetic construct
DIELTQPPSVSVAPGQTARISCSGDSIPNYYVYWYQQKPGQAPVLVIYDDSNRPSGIPERFSGSNSGNTATLTISGTQAEDEADYYCQSFDSSLNAEVFGGGTKLTVL

SEQ ID NO: 29 - protein, 4 aa, synthetic construct
NYIS

SEQ ID NO: 30 - protein, 17 aa, synthetic construct
IIDPDDSYTEYSPSFQG

SEQ ID NO: 31 - protein, 8 aa, synthetic construct
YEYGGFDI

SEQ ID NO: 32 - protein, 11 aa, synthetic construct
SGDNIGNSYVH

SEQ ID NO: 33 - protein, 7 aa, synthetic construct
KDNDRPS

SEQ ID NO: 34 - protein, 9 aa, synthetic construct
GTYDIESYV

SEQ ID NO: 35 - protein, 116 aa, synthetic construct
EVQLVQSGAEVKKPGESLKISCKGSGYSFTNYISWVRQMPGKGLEWMGIIDPDDSYTEYSPSFQGQVTISADKSISTAYLQWSSLKASDTAMYYCARYEYGGFDIWGQGTLVTVSS

SEQ ID NO: 36 - protein, 106 aa, synthetic construct
SYELTQPPSVSVAPGQTARISCSGDNIGNSYVHWYQQKPGQAPVLVIYKDNDRPSGIPERFSGSNSGNTATLTISGTQAEDEADYYCGTYDIESYVFGGGTKLTVL

SEQ ID NO: 37 - protein, 6 aa, synthetic construct
SSYYVA

SEQ ID NO: 38 - protein, 17 aa, synthetic construct
AIYTGSGATYKASWAKG

SEQ ID NO: 39 - protein, 13 aa, synthetic construct
DGGYDYPTHAMHY

SEQ ID NO: 40 - protein, 11 aa, synthetic construct
QASQNIGSSLA

SEQ ID NO: 41 - protein, 7 aa, synthetic construct
GASKTHS

SEQ ID NO: 42 - protein, 12 aa, synthetic construct
QSTKVGSSYGNH

SEQ ID NO: 43 - protein, 123 aa, synthetic construct
QVQLVESGGGLVQPGGSLRLSCAASGFTSHSSYYVAWVRQAPGKGLEWVGAIYTGSGATYKASWAKGRFTISKDTSKNQVVLTMTNMDPVDTATYYCASDGGYDYPTHAMHYWGQGTLVTVSS

SEQ ID NO: 44 - protein, 110 aa, synthetic construct
DVVMTQSPSSLSASVGDRVTITCQASQNIGSSLAWYQQKPGQAPRLLIYGASKTHSGVPSRFSGSGSGTDFTLTISSLQPEDVATYYCQSTKVGSSYGNHFGGGTKVEIK

SEQ ID NO: 45 - protein, 451 aa, synthetic construct
QVQLVESGGGLVQPGRSLRLSCAASGFTVHSSYYMAWVRQAPGKGLEWVGAIFTGSGAEYKAEWAKGRVTISKDTSKNQVVLTMTNMDPVDTATYYCASDAGYDYPTHAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELRRGPKVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTRKELSLSP

SEQ ID NO: 46 - protein, 217 aa, synthetic construct
DIQMTQSPSSLSASVGDRVTITCRASQGISSSLAWYQQKPGKAPKLLIYGASETESGVPSRFSGSGSGTDFTLTISSLQPEDFATYYCQNTKVGSSYGNTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 120 aa, synthetic construct
QVQLQESGPGLVKPSETLSLTCTVSGDSVSSSYWTWIRQPPGKGLEWIGYIYYSGSSNYNPSLKSRATISVDTSKNQFSLKLSSVTAADTAVYYCAREGNVDTTMIFDYWGQGTLVTVSS

SEQ ID NO: 48 - protein, 107 aa, synthetic construct
AIQMTQSPSSLSASVGDRVTITCRASQGIRNDLGWYQQKPGKAPKLLIYAASSLQSGVPSRFAGRGSGTDFTLTISSLQPEDFATYYCLQDFNYPWTFGQGTKVEIK

SEQ ID NO: 49 - protein, 447 aa, synthetic construct
QVQLQESGPGLVKPSETLSLTCTVSGDSVSSSYWTWIRQPPGKGLEWIGYIYYSGSSNYNPSLKSRATISVDTSKNQFSLKLSSVTAADTAVYYCAREGNVDTTMIFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 50 - protein, 214 aa, synthetic construct
AIQMTQSPSSLSASVGDRVTITCRASQGIRNDLGWYQQKPGKAPKLLIYAASSLQSGVPSRFAGRGSGTDFTLTISSLQPEDFATYYCLQDFNYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC